(19) United States
(12) Patent Application Publication
(10) Pub. No.: US 2016/0143884 A1
(43) Pub. Date: May 26, 2016

(54) USE OF TETRAHYDROINDAZOLYLBENZAMIDE AND TETRAHYDROINDOLYLBENZAMIDE DERIVATIVES FOR THE TREATMENT OF HUMAN IMMUNODEFICIENCY VIRUS (HIV) AND ACQUIRED IMMUNE DEFICIENCY SYNDROME (AIDS)

(71) Applicants: Esanex, Inc., Indianapolis, IN (US); Ohio State Innovation Foundation, Columbus, OH (US); Duke University, Durham, NC (US)

(72) Inventors: Everardus O.M. Orlemans, Chapel Hill, NC (US); Barton F. Haynes, Durham, NC (US); Guido Ferrari, Durham, NC (US); Timothy Haystead, Durham, NC (US); Jesse John Kwiek, Worthington, OH (US)

(21) Appl. No.: 14/952,421

(22) Filed: Nov. 25, 2015

Related U.S. Application Data
(60) Provisional application No. 62/085,062, filed on Nov. 26, 2014.

(51) Int. Cl. A61K 31/416; A61K 45/06

(52) U.S. Cl. CPC A61K 31/416; A61K 45/06

(57) ABSTRACT

Provided are methods of treating or inhibiting Human Immunodeficiency Virus (HIV) infection, or treating or inhibiting Acquired Human Immunodeficiency Syndrome (AIDS) in a subject in need thereof, comprising administering an Hsp90 inhibitor in a therapeutically effective amount.

### CROSS-REFERENCE TO RELATED APPLICATIONS

This application claims the benefit of the filing date of U.S. provisional application No. 62/085,062, filed Nov. 26, 2014, which is incorporated by reference herein in its entirety.

### BACKGROUND OF THE INVENTION

1. Field of the Invention

The invention relates to therapies useful in treating or inhibiting Human Immunodeficiency Virus (HIV) infection, or treating or inhibiting Acquired Human Immunodeficiency Syndrome (AIDS).

2. Description of the Related Art

While human immunodeficiency virus (HIV) infection, which results in AIDS, is a relatively new infection in the human population, it has quickly risen to the foremost health problem in the world. HIV/AIDS is now the leading cause of death in sub-Saharan Africa, and is the fourth biggest killer worldwide. While better treatment methods are now known to prolong the life of patients with HIV infection, there is still no cure.

In initial stage, many people will not have any symptoms when they first become infected with HIV. They may, however, have a flu-like illness within a month or two after exposure to the virus. This illness may include fever, headache, profound weakness, enlarged lymph nodes (glands of the immune system easily felt in the neck and groin) these symptoms usually disappear within a week to a month and are often mistaken for those of another viral infection. During this period, people are very infectious, and HIV is present in large quantities in genital fluids. More persistent or severe symptoms may not appear for 10 years or more after HIV first enter the body in adults, or within 2 years in children born with HIV infection. This period of asymptomatic infection varies greatly in each person. Some people may begin to have symptoms within a few months, while others may be symptom-free for more than 10 years.

HIV is a member of the class of viruses known as retroviruses. The retroviral genome is composed of RNA, which is converted to DNA by reverse transcription. This retroviral DNA is then stably integrated into a host cell's chromosome and, employing the replicative processes of the host cells, produces new retroviral particles and advances the infection to other cells. Even during the asymptomatic period, the virus is actively multiplying, infecting, and killing cells of the immune system. The virus can also hide within infected cells and be inactive. HIV appears to have a particular affinity for the human T-4 (e.g., CD4+ T-cell) lymphocyte cell, which plays a vital role in the body's immune system. HIV infection of these white blood cells depletes this white cell population. Eventually, the immune system is rendered inoperative and ineffective against various opportunistic diseases such as, among others, pneumocystic pneumonia, Karposi sarcoma, and cancer of the lymph system.

Although the exact mechanism of the formation and working of the HIV virus is not understood, identification of the virus has led to some progress in controlling the disease. For example, the use of anti-retroviral agents inhibit the reverse transcription of the retroviral genome of the HIV virus, thus giving a measure of control, though not a cure, for patients afflicted with AIDS.

Conventional antivirals often target the activities of specific viral enzymes. These approaches have been ineffective in stemming the emergence of drug-resistant variants, especially in the face of rapidly-mutating RNA viruses. In order to replicate, HIV, like all viruses, must use host-cellular machinery and induce production of viral genomic material, viral proteins and ultimately new virions. This hijacking and control over host cell processes is mediated by HIV proteins through a complex network of molecular events, including virus-host protein-protein interactions. In principle, disruptions to host-pathogen interactions would impede the propagation of pathogens. The identification of HIV dependency factors (HDFs) or “host cellular factors” highlights this point. HDFs represent a class of host proteins that are essential for HIV replication. Due to their evolutionarily resilient nature, targeting host proteins as a potential mechanism to treat HIV would prevent the emergence of drug-resistant HIV variants.

Heat-shock protein 90 (Hsp90) is a molecular chaperone that guides the folding, intracellular disposition, and proteolytic turnover of many key regulators of cell growth and differentiation. Hsp90 has a specific set of client proteins in vivo such as steroid receptors, transcription factors, protein kinases, and oncogenes. Inhibitors of Hsp90 have proven effective at driving cancer cells into apoptosis by preventing the proper folding of oncogenes required for promoting cancer cell growth. Because of this, several Hsp90 inhibitors are now in phase I and II clinical trials. Recently, Hsp90 was shown to be an important host factor for the replication of negative strand viruses. In addition, the inhibition of Hsp90 has been shown to block vaccinia virus replication by interaction with the viral core protein 4a in the cytoplasm. In the hepatitis C virus life cycle, Hsp90 is needed for proper cleavage of newly synthesized hepatitis C NSP2/3 protein and activity of hepatitis B reverse transcriptase. In polio virus, Hsp90 is required for proper folding of the viral capsid protein and Hsp90 inhibitors showed antiviral activity.

Hsp90 has been shown to control viral polymerase function for several viruses. For influenza virus, Hsp90 binds to the PB2 subunit of the RNA polymerase and stimulates its activity. In herpes viruses, blocking Hsp90 significantly inhibits viral replication presumably due to improper localization of the viral polymerase. In flock house virus, Hsp90 activity has proved to be important for stability and localization of the RNA polymerase. Recently, it was reported that Hsp90 inhibitors impaired the replication of several prototype negative-strand RNA viruses: vesicular stomatitis virus, Paramyxovirus (SVS, HPIV-2 & 3, SV41), and a bunyavirus (La Crosse), by destabilization of the L protein of the viral RDRP. It is thought that viruses have evolved to require the use of Hsp90 for proper folding of their RDRPs.

### SUMMARY OF THE INVENTION

The inventors have discovered that the Hsp90 inhibitors of this disclosure significantly inhibit the replication of Human Immunodeficiency Virus 1 (HIV-1), indicating that the inhibitors are useful as a therapeutic. Surprisingly, the Hsp90 inhibitors of this disclosure inhibit integration of HIV viral DNA into host cell and also inhibit Tat-mediated transactivation. Further, the Hsp90 inhibitors of this disclosure promote reactivation of latent HIV provirus. Significantly, the Hsp90 inhibitors of this disclosure were essentially inactive against hepatitis B virus (HBV). Instead, the Hsp90 inhibitors of the disclosure unexpectedly have potent anti-HIV activity.

Thus, in broad aspect, the invention encompasses methods of treating or inhibiting Human Immunodeficiency Virus (HIV) infection, or treating or inhibiting Acquired Human Immunodeficiency Syndrome (AIDS) in a subject in need thereof, comprising administering to the subject a therapeutically effective amount of an Hsp90 inhibitor, or a pharmaceutically acceptable salt thereof.

In one aspect, the invention encompasses methods of treating or inhibiting HIV infection, and/or treating or inhibiting AIDS in a subject in need thereof, comprising administering to the subject a therapeutically effective amount of an Hsp90 inhibitor is of formula (I):

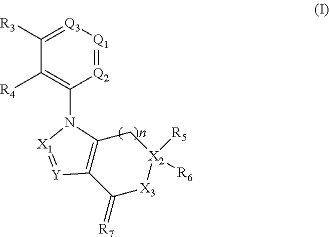

or a pharmaceutically acceptable salt thereof. In one embodiment of the methods of the disclosure, the treatment or inhibition of HIV infection, and/or the treatment or inhibition of AIDS is by: inhibiting integration of HIV viral DNA into a host cell, or inhibiting Tat-mediated transactivation of HIV DNA, or both. In one embodiment of the methods of the disclosure, the treatment or inhibition of HIV infection, and/or the treatment or inhibition AIDS is by: a) reactivating a latent HIV provirus in a host cell, and b) by inhibiting integration of HIV viral DNA into a host cell and/or by inhibiting Tat-mediated transactivation of HIV DNA.

In another aspect, the invention encompasses methods of inhibiting integration of HIV viral DNA into a host cell of a subject, comprising administering to the subject a therapeutically effective amount of an Hsp90 inhibitor is of formula (I).

In another aspect, the invention encompasses methods of inhibiting Tat-mediated transactivation of HIV DNA in a subject, comprising administering to the subject a therapeutically effective amount of an Hsp90 inhibitor is of formula (I).

In another aspect, the invention encompasses methods of reactivating a latent HIV provirus in a host cell, in combination with (i.e., concurrently or subsequently) inhibiting integration of HIV viral DNA into a host cell and/or inhibiting Tat-mediated transactivation of HIV DNA in a subject, comprising administering to the subject a therapeutically effective amount of an Hsp90 inhibitor is of formula (I).

The invention also provides the use of an HsP90 inhibitor described herein for the manufacture of a medicament for use in treating or inhibiting HIV infection, and/or treating or inhibiting AIDS.

Traditional antiretroviral therapies cannot eradicate HIV because the virus can become transcriptionally inactive in resting memory CD4+ T cells (and other cell types), which are long-lived, thus generating a reservoir undetectable by the immune system. When therapy is stopped, the latent viral reservoir can be reactivated and HIV rebounds. As a result, therapies with potential to both reactivate HIV from latency and inhibit the replication of HIV are highly desirable. The Hsp90 inhibitors of this disclosure also interfere with Tat's gene silencing process thereby causing modest reactivation of HIV. However, the Hsp90 inhibitors disclosed herein do not inhibit TNFα or phorbol ester induced reactivation HIV-1 reactivation. This result is surprising in view of report by Anderson et al. (Proc Natl Acad Sci USA; 111 (15):E1528-37 (2014)) that Hsp90 controls HIV reactivation, and that AUY922 (which is a Hsp90 inhibitor) almost completely suppressed HIV reactivation. Thus, in another aspect, the invention encompasses methods for reactivation of latent HIV provirus in a subject in need thereof, the method comprising administering to the subject a therapeutically effective amount of an Hsp90 inhibitor of formula (I):

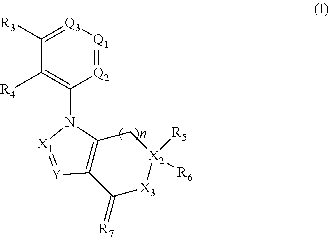

or a pharmaceutically acceptable salt thereof.

### BRIEF DESCRIPTION OF THE DRAWINGS

FIG. 1 provides a dose dependent evaluation of HIV-1_{Ba-L} replication in peripheral blood mononuclear cells (PBMCs) of (A) AZT (Zidovudine or azidothymidine), and (B) Compound 9 (4-(6,6-Dimethyl-4-oxo-3-trifluoromethyl-4,5,6,7-tetrahydro-indazol-1-yl)-2-(trans-4-hydroxy-cyclohexylamino)-benzamide).

FIG. 2 provides Compound 9 plasma concentration levels at different doses. Mean plasma concentration-time profile for escalating dose levels of after first dose (semi-log scale) on (A) cycle 1, day 1 and (B) cycle 1, days 15/18. Error bars are not shown for clarity.

FIG. 3 shows a dose dependent evaluation of Compound 9 for SIV inhibition in (non-human primate) NHP PBMC cells. (A) is a dose-dependent reduction of the amount of p27 in the supernatant of the cultures. (B) and (C) are the percentage of inhibition in experiments 1 and 2, respectively.

FIG. 4 provides evaluation of HIV-1 replication in SupT1 cells with raw data plotted on a log₁₀ scale.

FIG. 5 shows the % activity following Compound 9 treatment in SupT1 cells.

FIG. 6 shows the evaluation of HIV-1 reactivation in two JLAT clones (A2; 9.2) incubated for 24 hours with tumor necrosis factor alpha (TNFα) or phorbol myristate 13-acetate (PMA) to reactivate HIV-1 from latency. The graphs are plotted on a log₁₀ scale, with horizontal lines at 1%, 5%, 10%, and 50%.

FIG. 7 shows the Compound 9-mediated HIV reactivation in JLAT (A2) model; data represents percent GFP-positive cells (as determined by flow cytometry).

FIG. 8 shows evaluation of TAT-mediated transactivation in SupT1 cells were transfected with a HIV-LTR-Luc, +/−Tat (pcTAT, subtype B), and renilla-luciferase construct.

FIG. 9 shows evaluation of HIV-integration in a cell based assay with ALU-PCR read out.

FIG. 10 shows evaluation of HIV-integration in a cell based assay with 2LTR read out. (A) each of the GAPDH-normalized values to the no HIV 24 h sample; error bars represent standard deviation. (B) each of the previously normalized values to the HIV only 24 h sample; error bars represent standard deviation.

FIG. 11 shows percent inhibition of NL4-3-infected TZM-bl cells treated with nevirapine, raltegravir to three wells, and Compound 9.

FIG. 12 shows effect of Compound 9 on virus like particles (VLP) production

### DETAILED DESCRIPTION OF THE INVENTION

Before the disclosed methods are described, it is to be understood that the aspects described herein are not limited to specific embodiments, or compositions, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivative or pro-drug, such as pharmaceutically acceptable salts and esters. The manufacture of pharmaceutical compositions for different routes of administration falls within the capabilities of a person skilled in medicinal chemistry.

In view of the present disclosure, the methods described herein can be configured by the person of ordinary skill in the art to meet the desired need. In general, the disclosed methods provide improvements in the treatment and/or inhibition of HIV infection, or treatment and/or inhibition of AIDS. For example, in particular embodiments, the Hsp90 inhibitors of the disclosure (e.g., Compound 9) effectively inhibit HIV-1 replication in human peripheral blood mononuclear cells (PBMCs). Surprisingly, Compound 9 inhibits HIV-1 replication in PBMCs more efficiently than the commercially available antiviral drug Zidovudine (AZT). Compound 9 was shown to inhibit integration of HIV-1 viral DNA into host cell and inhibit Tat-mediated transactivation. Vozzolo, on the other hand, reported that two inhibitors of Hsp90: geldanamycin (GA) and 17-allylamino-17-demethoxy-geldanamycin (17-AAG) had no effect on integration. (see Vozzolo et al., J. Biol. Chem.; 285:39314-39328 (2010)).

Moreover, the Hsp90 inhibitors of this disclosure (e.g., Compound 9) unexpectedly interfere with Tat's gene silencing process thereby causing modest reactivation of HIV-1. However, Compound 9 does not inhibit TNFα or phorbol ester induced HIV-1 reactivation. This result is surprising in view of report by Anderson et al. (Proc Natl Acad Sci USA; 111 (15):E1528-37 (2014)) that Hsp90 controls HIV-1 reactivation, and that AUY922 (which is a Hsp90 inhibitor) almost completely suppressed HIV-1 reactivation.

In some embodiments of this disclosure, the subject in need is a human subject or patient. In some embodiments the subject, e.g., a human, has been previously treated with an antiviral therapy. In some other embodiments the subject has not been previously treated with an antiviral therapy.

The methods of the disclosure require an Hsp90 inhibitor or a pharmaceutically acceptable salt thereof. Numerous Hsp90 inhibitors are known in the art. Some Hsp90 inhibitors are disclosed in, for example, International Publication Nos. WO 2006/091963, WO 2007/101156, and WO 2008/130879, all incorporated herein by reference.

In one embodiment, the Hsp90 inhibitor useful in the methods and compositions of the disclosure is of formula (I):

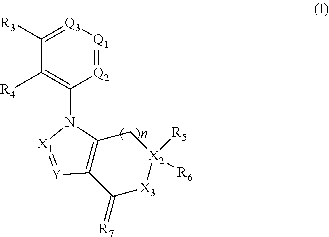

or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
   R₂₂ is
      (i) heteroaryl,
      (ii) aryl,
      (iii) saturated or unsaturated C₃-C₁₀ cycloalkyl, or
      (iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
   each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
   Z is OR₀ or —N(R₃₀)₂, wherein
      each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
      R₀ is —H, —C₁-C₁₀ alkyl, —C₂-C₁₀ alkenyl, —C₂-C₁₀ alkynyl, aryl, heteroaryl, or —C₁-C₆ acyl;
   R₂₃ is
      (1) heteroaryl,
      (2) aryl,
      (3) saturated or unsaturated C₅-C₁₀ cycloalkyl, or
      (4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is:

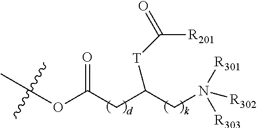

wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
   each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
      each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
         the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
      R_{C′} is —C₁-C₆ alkyl, —OR_{C″}, or —N(R_{CN})₂, wherein
         R_{C″} is —H, C₁-C₁₀ alkyl, C₁-C₁₀ haloalkyl, C₃-C₇ cycloalkyl, heterocycloalkyl, aryl, or heteroaryl;
         each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
         each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
   each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
   R₂₁ is cyano, —C(O)OH, —C(O)—O(C₁-C₆ alkyl), or a group of the formula

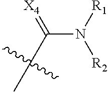

wherein
   R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
      each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
   X₂ and X₃ are independently C, O, N, or S(O)ₚ wherein
p is 0, 1, or 2; and
   n is 0, 1, 2, 3, or 4;
   provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
   R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

In Formula I, R₃ and R₄ are, as noted above, independently (a) hydrogen, (b) halo, or (c) an alkyl group having from 1-15 carbon atoms. All, but no more than about six, of the carbon atoms in the alkyl group may be replaced independently by the various groups listed above in connection with Formula I.

Thus, when the alkyl group is methyl, i.e., a one carbon atom alkyl group, replacement of that carbon atom with, for example, nitrogen or sulfur, the resulting group will not be an alkyl group but instead will be an amino or thio group, respectively. Similarly, when the carbon atom being replaced terminates the alkyl group, the terminal group will become another moiety such as pyrimidinyl, amino, phenyl, or hydroxy.

Replacement of a carbon atom with a group such as, for example, oxygen, nitrogen, or sulfur will require appropriate adjustment of the number of hydrogens or other atoms required to satisfy the replacing atom's valency. Thus, when the replacement is N or O, the number of groups attached to the atom being replaced will be reduced by one or two to satisfy the valency of the nitrogen or oxygen respectively. Similar considerations will be readily apparent to those skilled in the art with respect to replacement by ethenyl and ethynyl.

Thus, replacement as permitted herein results in the term “C₁-C₁₅ alkyl” as defined in connection with Formula I encompassing groups such as, but not limited to: amino, hydroxy, phenyl, benzyl, propylaminoethoxy, butoxyethylamino, pyrid-2-ylpropyl, diethylaminomethyl, pentylsulfonyl, methylsulfonamidoethyl, 3-[4-(butylpyrimidin-2-yl)ethyl]phenyl, butoxy, dimethylamino, 4-(2-(benzylamino)ethyl)pyridyl, but-2-enylamino, 4-(1-(methylamino)pent-3-en-2-ylthio)phenyl, 2-(N-methyl-hexanamido)ethoxy)methyl, and 4-(((3-methoxy-4-(4-methyl-1H-imidazol-2-yl)but-1-enyl)(methyl)aminoymethyl)phenyl.

Preferred compounds of Formula I include those where X₁ is carbon optionally substituted with C₁-C₆ alkyl, more preferably C₁-C₃ alkyl. Other preferred compounds of Formula I are those where X₁ is carbon optionally substituted with C₁-C₆ alkyl and Y is CR_{C} wherein R_{C} is —H, C₁-C₆ alkyl, C₁-C₃ haloalkyl, C₃-C₇ cycloalkyl, or C₃-C₇ cycloalkyl(C₁-C₆)alkyl. More preferably, in compounds of Formula I, X₁ is carbon optionally substituted with C₁-C₂ alkyl and Y is CR_{C} wherein R_{C} is —H, C₁-C₄ alkyl, C₁-C₃ haloalkyl, cyclopropyl, or cyclopropyl(C₁-C₂)alkyl.

Still more preferred compounds of Formula I are those where X₁ is CH. Other more preferred compounds of Formula I are those where X₁ is CH and Y is CR_{C} wherein R_{C} is —H, C₁-C₃ alkyl, C₁-C₃ haloalkyl, C₃-C₅ cycloalkyl, or C₃-C₅ cycloalkyl(C₁-C₂)alkyl. Even more preferred compounds of Formula I are those where X₁ is CH and Y is CR_{C} wherein R_{C} is —H, methyl, ethyl, trifluoromethyl, cyclopropyl, or cyclopropylmethyl. Particularly preferred compounds of Formula I are those where X₁ is CH and Y is CR_{C} wherein R_{C} is methyl, ethyl, or cyclopropyl. Other particularly preferred compounds of Formula I are those where X₁ is CH and Y is CR_{C} wherein R_{C} is trifluoromethyl. Other particularly preferred compounds of Formula I are those where X₁ is CH and Y is CR_{C} wherein R_{C} is methyl. Other particularly preferred compounds of Formula I are those where X₁ is CH and Y is CR_{C} wherein R_{C} is ethyl. Other particularly preferred compounds of Formula I are those where X₁ is CH and Y is CR_{C} wherein R_{C} is cyclopropyl.

Still more preferred compounds of Formula I are those where X₁ is N. Other more preferred compounds of Formula I are those where X₁ is N and Y is CR_{C} wherein R_{C} is —H, C₁-C₃ alkyl, C₁-C₃ haloalkyl, C₃-C₅ cycloalkyl, or C₃-C₅ cycloalkyl(C₁-C₂)alkyl. Even more preferred compounds of Formula I are those where X₁ is N and Y is CR_{C} wherein R_{C} is —H, methyl, ethyl, trifluoromethyl, cyclopropyl, or cyclopropylmethyl. Particularly preferred compounds of Formula I are those where X₁ is N and Y is CR_{C} wherein R_{C} is methyl, ethyl, or cyclopropyl. Other particularly preferred compounds of Formula I are those where X₁ is N and Y is CR_{C} wherein R_{C} is trifluoromethyl. Other particularly preferred compounds of Formula I are those where X₁ is N and Y is CR_{C} wherein R_{C} is methyl. Other particularly preferred compounds of Formula I are those where X₁ is N and Y is CR_{C} wherein R_{C} is ethyl. Other particularly preferred compounds of Formula I are those where X₁ is N and Y is CR_{C} wherein R_{C} is cyclopropyl.

Other preferred compounds of Formula I are those where

Q₃ is CR₂₁, wherein

R₂₁ is a group of the formula,

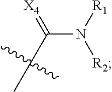

R₇ is O; and

Y is CR_{C}, wherein Rₑ is hydrogen, C₁-C₃ alkyl, C₃-C₅ cycloalkyl, trifluoromethyl, or C₃-C₅ cycloalkyl(C₁-C₂)alkyl. Such compounds are compounds of Formula II herein.

Other preferred compounds of Formula I are those where

Q₃ is CR₂₁, wherein

R₂₁ is a group of the formula,

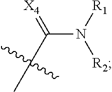

and
X₃ is C substituted with R₉ₐ and R_{9b}, wherein R₉ₐ and R_{9b} are independently H or C₁-C₆ alkyl.
Such compounds are hereinafter compounds of Formula III.

Other preferred compounds of Formula I are those where Q₃ is CR₂₁, wherein

R₂₁ is a group of the formula,

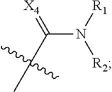

and
Q₁ and Q₂ are independently C substituted with R₁₀ₐ and R_{10b} respectively, wherein R₁₀ₐ and R_{10b} are independently H or C₁-C₆ alkyl. Such compounds are hereinafter compounds of Formula IV.

Other preferred compounds of Formula I are those where

Q₃ is CR₂₁, wherein

R₂₁ is a group of the formula,

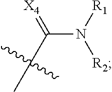

and
X₁ is C substituted with R₁₁ where R₁₁ hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
   R_{C″} is —C₁-C₆ alkyl, —OR_{C″}, or —N(R_{CN})₂, wherein
      R_{C″} is —H, C₁-C₁₀ alkyl, C₁-C₁₀ haloalkyl, C₃-C₇ cycloalkyl, heterocycloalkyl, aryl, or heteroaryl;
         each R_{CN} is independently —H, —C₁-C₁₀ alkyl, —C₁-C₁₀-haloalkyl, —C₃-C₇ cycloalkyl, -heterocycloalkyl, —C₁-C₆ acyl, -aryl, or -heteroaryl. Such compounds are hereinafter compounds of Formula V.

Preferred compounds of Formula V are those where R₁₁ is hydrogen, halogen, C₁-C₁₀ alkyl, C₁-C₁₀ haloalkyl, C₃-C₇ cycloalkyl, C₃-C₇ cycloalkyl(C₁-C₁₀)alkyl, aryl, or heteroaryl.

More preferred compounds of Formula V are those where R₁₁ is H or C₁-C₆ alkyl.

Other preferred compounds of Formula I are those where

Q₃ is CR₂₁, wherein

R₂₁ is a group of the formula,

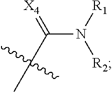

and
X₁ is N. Such compounds are hereinafter compounds of Formula Va.

Other preferred compounds of Formula I are those where

Q₃ is CR₂₁, wherein

R₂₁ is a group of the formula,

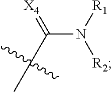

and
X₂ is C substituted with R₅ and R₆, wherein R₅ and R₆ are independently H or C₁-C₄ alkyl. Such compounds are hereinafter compounds of Formula VI.

More preferred compounds of the invention are those of Formula I wherein Q₃ is CR₂₁, wherein

More preferred compounds of the invention are those of Formula I wherein

R₁ and R₂ are independently H or C₁-C₄ alkyl;

Q₁ and Q₂ are both CH;

X₂ is C substituted with two independently selected C₁-C₄ alkyl groups; and

n is 1.

Other preferred compounds of the invention include those having the formula VII

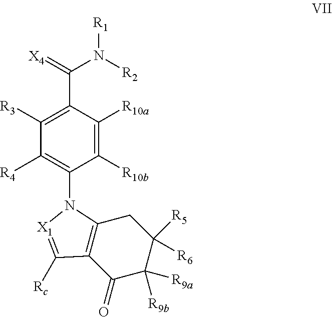

wherein X₁ and R_{C} are as defined in Formula I;
R₅ and R₆ are independently H or C₁-C₄ alkyl;
R₁₁ is H or C₁-C₆ alkyl;
R₁₀ₐ and R_{10b} are independently H or C₁-C₆ alkyl;
R₉ₐ and R_{9b} are independently H or C₁-C₆ alkyl.

Preferred compounds of Formula VII include those where

R₁ and R₂ are independently H or C₁-C₄ alkyl;
R₁₀ₐ and R_{10b} are both H; and
R₅ and R₆ are independently C₁-C₄ alkyl.

Other preferred compounds of Formula VII include those where X₁ is N.

Other preferred compounds of Formula VII include those where X₁ is CR_{C}, wherein R_{C} is hydrogen, methyl, ethyl, cyclopropyl, cyclopropylmethyl, fluoromethyl, difluoromethyl, or trifluoromethyl. In a preferred embodiment of this aspect, the R_{C} group derived from X₁ is hydrogen, methyl, or trifluoromethyl, and the R_{C} group derived from Y carries the definition given in connection with Formula I.

Other preferred compounds of Formula I include those of formula VIII,

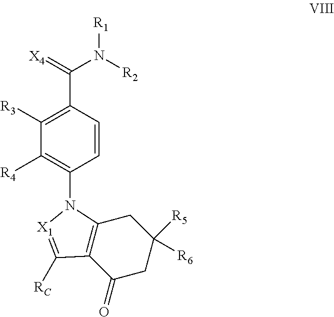

wherein R_{C} is H, C₁-C₆ alkyl, trifluoromethyl, or cyclopropyl; and
R₁-R₆, X₁, and X₄ carry the same definitions as for Formula I.

Preferred compounds of Formula VIII include those where X₁ is N.

Preferred compounds of Formula VIII include those where X₁ is CR_{C}, wherein R_{C} is hydrogen, methyl, ethyl, cyclopropyl, cyclopropylmethyl, fluoromethyl, difluoromethyl, or trifluoromethyl. In a preferred embodiment of this aspect, the R_{C} group derived from X₁ is hydrogen, methyl, or trifluoromethyl, and the R_{C} group derived from Y carries the definition given in connection with Formula I.

Other preferred compounds of Formula I are those of Formula IX:

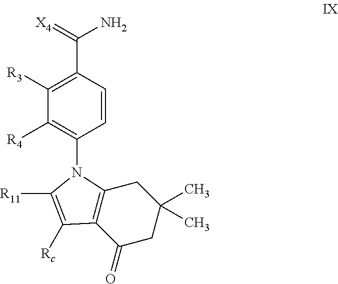

where R₁₁ is hydrogen or methyl, preferably hydrogen;
R_{C} is H, C₁-C₂ alkyl, trifluoromethyl, or cyclopropyl; and
R₃, R₄, and X₄ carry the same definitions as for Formula I. Preferred compounds of Formula IX include those where R_{C} is C₁-C₂ alkyl, trifluoromethyl, or cyclopropyl.

Other preferred compounds of Formula I are those where R₂₁ is cyano, R₇ is O, and Y is CR_{C}, wherein R_{C} is H, methyl, ethyl, trifluoromethyl, or cyclopropyl.

Other preferred compounds of Formula I are those where,

R₂₁ is cyano; R₇ is O; and Y is CR_{C}, wherein R_{C} is H, methyl, trifluoromethyl, or cyclopropyl.

Yet other preferred compounds of Formula I are those where R₂₁ is cyano, and X₃ is C substituted with two groups that are independently H or C₁-C₆ alkyl.

More preferred compounds of Formula I are those where R₂₁ is cyano, and Q₁ and Q₂ are independently C substituted with H or C₁-C₆ alkyl.

Yet other preferred compounds of Formula I are those where R₂₁ is cyano, and X₁ is C substituted with H or C₁-C₆ alkyl.

Still other preferred compounds of Formula I are those where R₂₁ is cyano, and X₂ is C substituted with two groups that are independently H or C₁-C₄ alkyl.

Yet other preferred compounds of Formula I include those of Formula X,

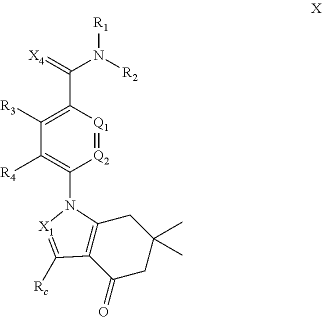

wherein X₁-X₄, Q₁, Q₂, R_{C}, and R₁-R₄ are as defined in Formula I.

Preferred compounds of formula X are those where Q₁ and Q₂ are each independently hydrogen or C₁-C₆ alkyl.

Other preferred compounds of formula X are those where R_{C} is C₁-C₆alkyl, C₃-C₇cycloalkyl, C₁-C₆ haloalkyl, C₃-C₇cycloalkyl(C₁-C₆)alkyl, or heterocycloalkyl.

More preferred compounds of Formula X include those where R_{C} is C₃-C₇cycloalkyl, C₁-C₆ haloalkyl, heterocycloalkyl, or C₃-C₇cycloalkyl(C₁-C₆)alkyl.

Particularly preferred compounds of Formula X include those where R_{C} is C₁-C₃alkyl, C₃-C₅cycloalkyl, C₃-C₅cycloalkyl(C₁-C₃)alkyl, or C₁-C₂ haloalkyl.

Preferred compounds of Formula X are those where X₁ is N. Such compounds are referred to herein as compounds of Formula XI.

Additional compounds of any of Formulas I-X include those wherein R₃ is substituted with R₅₀, and R₅₀ is

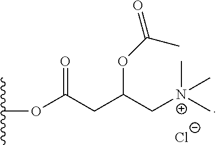 [SMILES: *OC(=O)CC(C[N+](C)(C)C)OC(C)=O ; [Cl-]]

Other preferred compounds of any of Formulas I-X include those wherein
Q₃ is CR₂₁, wherein
   R₂₁ is a group of the formula,

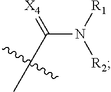

R₇ is O; and
Y is CR_{C}, wherein
   R_{C} is —H, —CH₃, ethyl, cyclopropyl, or —CF₃.

Other preferred compounds of any of Formulas I-X include those wherein
Q₃ is CR₂₁, wherein
   R₂₁ is a group of the formula,

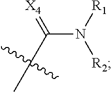

and X₂ is C substituted with two groups that are independently H or C₁-C₆ alkyl.

Other preferred compounds of any of Formulas I-X include those wherein Q₃ is CR₂₁, wherein
R₂₁ is a group of the formula,

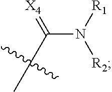

and Q₁ and Q₂ are independently C substituted with H or C₁-C₆ alkyl.

Other preferred compounds of any of Formulas I-X include those wherein X₁ is N.

Other preferred compounds of any of Formulas I-X include those wherein X₁ is CR_{C}.

Other preferred compounds of any of Formulas I-X include those wherein Q₃ is CR₂₁, wherein
R₂₁ is a group of the formula,

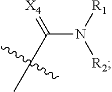

and X₁ is C substituted with H or C₁-C₆ alkyl.

Other preferred compounds of any of Formulas I-X include those wherein Q₃ is CR₂₁, wherein

R₂₁ is a group of the formula,

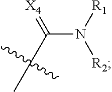

and X₂ is C substituted with two groups that are independently H or C₁-C₄ alkyl.

Other preferred compounds of any of Formulas I-X include those wherein R₂₁ is a group of the formula,

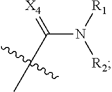

R₃ is —Z₁R_{Z1}, wherein
   Z₁ is —O— or —NH—; and
   R_{Z1} is a saturated or unsaturated C₃-C₁₀ cycloalkyl, each of which is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃; and
R₄ is H or halogen.

Other preferred compounds of any of Formulas I-X include those wherein R_{z1} is a saturated C₅-C₇ cycloalkyl.

Other preferred compounds of any of Formulas I-X include those wherein wherein R_{Z1} is a unsaturated C₅-C₇ cycloalkyl.

Other preferred compounds of any of Formulas I-X include those wherein X₁ is N.

Other preferred compounds of any of Formulas I-X include those wherein X₁ is CR_{C}.

Other preferred compounds of any of Formulas I-X include those wherein X₁ is CH.

Other preferred compounds of any of Formulas I-X include those wherein
R₁ and R₂ are independently H or C₁-C₄ alkyl;
Q₁ and Q₂ are both CH;
X₂ is C substituted with two independently selected C₁-C₄ alkyl groups; and
n is 1.

Other preferred compounds of any of Formulas I-X include those of the formula,

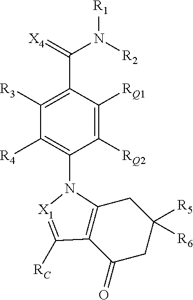

wherein

R_{Q1} is H or halogen; and

R_{Q2} is H or halogen.

Other preferred compounds of any of Formulas I-X include those wherein
R₃ is —Z₁-cyclohexyl which is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃; and R₄ is H or fluoro.

In some embodiments, the Hsp90 inhibitor is 4-(6,6-dimethyl-4-oxo-3-trifluoromethyl-4,5,6,7-tetrahydro-indazol-1-yl)-2-(trans-4-hydroxy-cyclohexylamino)-benzamide:

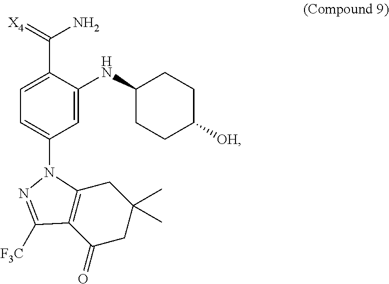

or
trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate:

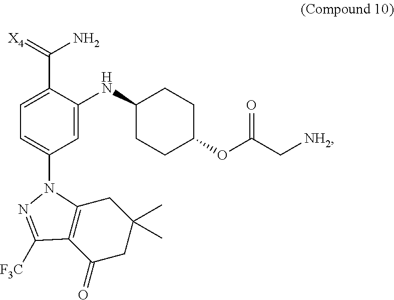

or pharmaceutically acceptable salts thereof. Synthesis and characterization data for these compounds are described in U.S. Pat. No. 7,358,370, which is incorporated by reference in its entirety.

Examples of Hsp90 inhibitors suitable for use in the methods and compositions of the disclosure include, but are not limited to compounds listed in Table 1.

TABLE 1
Hsp90 Inhibitors

| | |
|---|---|
| 1 | 2-((4-hydroxycyclohexyl)amino)-4-(3,6,6-trimethyl-4-oxo-4,5,6,7-tetrahydro-1H- |
| | indazol-1-yl)benzamide; |
| 2 | 2-((2-hydroxyethyl)amino)-4-(3,6,6-trimethyl-4-oxo-4,5,6,7-tetrahydro-1H-indazol-1- |
| | yl)benzamide; |
| 3 | 2-((2-methoxyethyl)amino)-4-(3,6,6-trimethyl-4-oxo-4,5,6,7-tetrahydro-1H-indazol-1- |
| | yl)benzamide; |
| 4 | 2-((2-ethoxyethyl)amino)-4-(3,6,6-trimethyl-4-oxo-4,5,6,7-tetrahydro-1H-indazol-1- |
| | yl)benzamide; |
| 5 | 2-((2-(2-(2-aminoethoxy)ethoxy)ethyl)amino)-4-(3,6,6-trimethyl-4-oxo-4,5,6,7- |
| | tetrahydro-1H-indazol-1-yl)benzamide; |
| 6 | 2-((3-hydroxypropyl)amino)-4-(3,6,6-trimethyl-4-oxo-4,5,6,7-tetrahydro-1H-indazol-1- |
| | yl)benzamide; |
| 7 | 2-((3-methoxypropyl)amino)-4-(3,6,6-trimethyl-4-oxo-4,5,6,7-tetrahydro-1H-indazol-1- |
| | yl)benzamide; |
| 8 | 2-((3-ethoxypropyl)amino)-4-(3,6,6-trimethyl-4-oxo-4,5,6,7-tetrahydro-1H-indazol-1- |
| | yl)benzamide; |
| 9 | 4-(6,6-dimethyl-4-oxo-3-trifluoromethyl-4,5,6,7-tetrahydro-indazol-1-yl)-2-(trans-4- |
| | hydroxy-cyclohexylamino)-benzamide; |
| 10 | trans-4-({2-(aminocarbonyl)-5-[6,6-dimethyl-4-oxo-3-(trifluoromethyl)-4,5,6,7- |
| | tetrahydro-1H-indazol-1-yl]phenyl}amino)cyclohexyl glycinate; |
| 11 | 2-bromo-4-(3,6,6-trimethyl-4-oxo-4,5,6,7-tetrahydro-indol-1-yl)-benzonitrile; |
| 12 | 2-(4-hydroxy-cyclohexylamino)-4-(3,6,6-trimethyl-4-oxo-4,5,6,7-tetrahydro-indol-1-yl)- |
| | benzamide; |
| 13 | 2-(tetrahydro-pyran-4-ylamino)-4-(3,6,6-trimethyl-4-oxo-4,5,6,7-tetrahydro-indol-1-yl)- |
| | benzamide; |
| 14 | 2-(2-methoxy-ethylamino)-4-(3,6,6-trimethyl-4-oxo-4,5,6,7-tetrahydro-indol-1-yl)- |
| | benzamide; |
| 15 | 2-(3,4,5-trimethoxy-phenylamino)-4-(3,6,6-trimethyl-4-oxo-4,5,6,7-tetrahydro-indol-1- |
| | yl)-benzamide; |
| 16 | 2-[(pyridin-3-ylmethyl)-amino]-4-(3,6,6-trimethyl-4-ox-4,5,6,7-tetrahydro-indol-1-yl)- |
| | benzamide; |
| 17 | 2-(4-oxo-cyclohexylamino)-4-(3,6,6-trimethyl-4-oxo-4,5,6,7-tetrahydro-indol-1-yl)- |
| | benzamide; |
| 18 | 2-[4-(2-morpholin-4-yl-ethoxy)-cyclohexylamino]-4-(3,6,6-trimethyl-4-oxo-4,5,6,7- |
| | tetrahydro-indol-1-yl)-benzamide; |
| 19 | 2-bromo-4-(3,6,6-trimethyl-4-oxo-4,5,6,7-tetrahydro-indazol-1-yl)-benzonitrile; |
| 20 | 2-(tetrahydro-pyran-4-ylamino)-4-(3,6,6-trimethyl-4-oxo-4,5,6,7-tetrahydro-indazol-1- |
| | yl)-benzamide; |
| 21 | 2-(tetrahydrothiopyran-4-ylamino)-4-(3,6,6-trimethyl-4-oxo4,5,6,7-tetrahydroindazol-1- |
| | yl)benzamide; |
| 22 | 2-(1-oxo-hexahydro-1-Tetrahydrothiopyran-4-ylamino)-4-(3,6,6-trimethyl-4-oxo4,5,6,7- |
| | tetrahydroindazol-1-yl)benzamide; |
| 23 | 2-(1,1-dioxo-hexahydro-1-Tetrahydrothiopyran-4-ylamino)-4-(3,6,6-trimethyl-4- |
| | oxo4,5,6,7-tetrahydroindazol-1-yl)benzamide; |
| 24 | 2-bromo-4-(6,6-dimethyl-4-oxo-3-trifluoromethyl-4,5,6,7-tetrahydro-indazol-1-yl)- |
| | benzonitrile; |
| 25 | 2-(cyclopent-3-enylamino)-4-(3,6,6-trimethyl-4-oxo-4,5,6,7-tetrahydro-indazol-1- |
| | yl)benzamide; |
| 26 | 4-(6,6-dimethyl-4-oxo-3-methyl-4,5,6,7-tetrahydro-indazol-1-yl)-2-(3,4,5- |
| | trimethoxyanilino)-benzamide; |
| 27 | amino-acetic acid 4-[2-carbamoyl-5-(3,6,6-trimethyl-4-oxo-4,5,6,7-tetrahydro-indol-1- |
| | yl)-phenylamino]-cyclohexyl ester; methanesulfonic acid salt; |
| 28 | dimethylamino-acetic acid 4-[2-carbamoyl-5-(3,6,6-trimethyl-4-oxo-4,5,6,7-tetrahydro- |
| | indol-1-yl)-phenylamino]-cyclohexyl ester; methanesulfonic acid salt; |
| 29 | 2-[3-(2-dimethylamino-ethoxy)-4-methoxy-phenylamino]-4-(6,6-dimethyl-4-oxo-3- |
| | trifluoromethyl-4,5,6,7-tetrahydro-indazol-1-yl)-benzamide; |
| 30 | 4-(6,6-dimethyl-4-oxo-3-trifluoromethyl-4,5,6,7-tetrahydro-indazol-1-yl)-2-(tetrahydro- |
| | pyran-4-ylamino)-benzamide; |
| 31 | 2-bromo-4-(3-difluoromethyl-6,6-dimethyl-4-oxo-4,5,6,7-tetrahydro-indazol-1-yl)- |
| | benzonitrile; |
| 32 | 4-(6,6-dimethyl-4-oxo-3-difluoromethyl-4,5,6,7-tetrahydro-indazol-1-yl)-2-(tetrahydro- |
| | pyran-4-ylamino)-benzamide; |
| 33 | 2-[3-(2-dimethylamino-ethoxy)-4-methoxy-phenylamino]-4-(3,6,6-trimethyl-4-oxo- |
| | 4,5,6,7-tetrahydro-indazol-1-yl)-benzamide; |
| 34 | 4-(3-cyclopropylmethyl-6,6-dimethyl-4-oxo-4,5,6,7-tetrahydro-indazol-1-yl)-benzamide; |
| 35 | 2-[2-(2-dimethylamino-ethoxy)-pyridin-4-ylamino]-4-(3,6,6-trimethyl-4-oxo-4,5,6,7- |
| | tetrahydro-indazol-1-yl)-benzamide, methansulfonic acid salt; |
| 36 | 2-(2,3-dihydroxy-propylamino)-4-(6,6-dimethyl-4-oxo-3-trifluoromethyl-4,5,6,7- |
| | tetrahydro-indazol-1-yl)-benzamide;; |
| 37 | 4-(6,6-Dimethyl-4-oxo-3-trifluoromethyl-4,5,6,7-tetrahydro-indazol-1-yl)-2-(tetrahydro- |
| | thiopyran-4-ylamino)-benzamide; |
| 38 | 4-(3-Difluoromethyl-6,6-dimethyl-4-oxo-4,5,6,7-tetrahydro-indazol-1-yl)-2-(tetrahydro- |
| | thiopyran-4-ylamino)-benzamide; |
| 39 | 2-[(Tetrahydro-furan-2-ylmethyl)-amino]-4-(3,6,6-trimethyl-4-oxo-4,5,6,7-tetrahydro- |
| | indazol-1-yl)-benzamide; |
| 40 | 2-[3-(2-Oxo-pyrrolidin-1-yl)-propylamino]-4-(3,6,6-trimethyl-4-oxo-4,5,6,7-tetrahydro- |
| | indazol-1-yl)-benzamide; |
| 41 | 2-(2,2,2-Trifluoro-ethylamino)-4-(3,6,6-trimethyl-4-oxo-4,5,6,7-tetrahydro-indazol-1-yl)- |
| | benzamide; |
| 42 | 2-(allylamino)-4-(3-methyl-4-oxo-4,5,6,7-tetrahydroindol-1-yl)benzamide; |
| 43 | 2-(cyclopropylamino)-4-(3-methyl-4-oxo-4,5,6,7-tetrahydroindol-1-yl)benzamide; |
| 44 | 2-(2-methoxyethylamino)-4-(3-methyl-4-oxo-4,5,6,7-tetrahydroindol-1-yl)benzamide; |
| 45 | 4-(3-methyl-4-oxo-4,5,6,7-tetrahydroindol-1-yl)benzamide; |
| 46 | 4-(3,6,6-trimethyl-4-oxo-4,5,6,7-tetrahydroindol-1-yl)benzamide; |
| 47 | 4-(3-methyl-4-oxo-4,5,6,7-tetrahydroindol-1-yl)-2-(phenylamino)benzamide; |
| 48 | 2-(4-hydroxycyclohexylamino)-4-(3-methyl-4-oxo-4,5,6,7-tetrahydroindol-1- |
| | yl)benzamide; |
| 49 | 4-(3-methyl-4-oxo-4,5,6,7-tetrahydroindol-1-yl)-2-(3,4,5- |
| | trimethoxyphenylamino)benzamide; |
| 50 | 2-(2-(dimethylamino)ethylamino)-4-(3,6,6-trimethyl-4-oxo-4,5,6,7-tetrahydroindol-1- |
| | yl)benzamide; |
| 51 | 2-(2-(dimethylamino)ethylamino)-4-(3-methyl-4-oxo-4,5,6,7-tetrahydroindol-1- |
| | yl)benzamide; |
| 52 | 2-(pyridin-4-ylmethylamino)-4-(3,6,6-trimethyl-4-oxo-4,5,6,7-tetrahydroindol-1- |
| | yl)benzamide; |
| 53 | 4-(3-methyl-4-oxo-4,5,6,7-tetrahydroindol-1-yl)-2-(pyridin-3-ylmethylamino)benzamide; |
| 54 | tert-butyl 4-(2-carbamoyl-5-(3,6,6-trimethyl-4-oxo-4,5,6,7-tetrahydroindol-1- |
| | yl)phenylamino)piperidine-1-carboxylate; |
| 55 | 2-amino-4-(3,6,6-trimethyl-4-oxo-4,5,6,7-tetrahydroindol-1-yl)benzamide; |
| 56 | 2-(allylamino)-4-(3,6,6-trimethyl-4-oxo-4,5,6,7-tetrahydroindol-1-yl)benzamide; |
| 57 | 2-(1-methylpiperidin-4-ylamino)-4-(3,6,6-trimethyl-4-oxo-4,5,6,7-tetrahydroindol-1- |
| | yl)benzamide; |
| 58 | 2-(piperidin-4-ylamino)-4-(3,6,6-trimethyl-4-oxo-4,5,6,7-tetrahydroindol-1- |
| | yl)benzamide; |
| 59 | 3-butoxy-4-(3,6,6-trimethyl-4-oxo-4,5,6,7-tetrahydroindol-1-yl)benzamide; |
| 60 | 2-(2,3-dihydro-1H-inden-1-ylamino)-4-(3,6,6-trimethyl-4-oxo-4,5,6,7-tetrahydroindol-1- |
| | yl)benzamide; |
| 61 | 1-(2-carbamoyl-5-(3,6,6-trimethyl-4-oxo-4,5,6,7-tetrahydroindol-1-yl)phenyl)urea; |
| 62 | 2-bromo-4-(2,6,6-trimethyl-4-oxo-4,5,6,7-tetrahydro-1H-indol-1-yl)benzonitrile; |
| 63 | 3-fluoro-4-(2,6,6-trimethyl-4-oxo-4,5,6,7-tetrahydro-1H-indol-1-yl)benzonitrile; |
| 64 | 3-butoxy-4-(2,6,6-trimethyl-4-oxo-4,5,6,7-tetrahydro-1H-indol-1-yl)benzamide; |
| 65 | 2-(phenylamino)-4-(2,6,6-trimethyl-4-oxo-4,5,6,7-tetrahydro-1H-indol-1-yl)benzamide; |
| 66 | 3-butoxy-N′-hydroxy-4-(2,6,6-trimethyl-4-oxo-4,5,6,7-tetrahydro-1H-indol-1- |
| | yl)benzimidamide; |
| 67 | 2-benzylamino-4-(2,6,6-trimethyl-4-oxo-4,5,6,7-tetrahydro-indol-1-yl)-benzamide; |
| 68 | 3-prop-2-ynyloxy-4-(2,6,6-trimethyl-4-oxo-4,5,6,7-tetrahydro-indol-1-yl)-benzamide; |
| 69 | 2-ethynyl-4-(2,6,6-trimethyl-4-oxo-4,5,6,7-tetrahydro-indol-1-yl)-benzamide; |
| 70 | 2-(4-Methoxy-phenylamino)-4-(2,6,6-trimethyl-4-oxo-4,5,6,7-tetrahydro-indol-1-yl)- |
| | benzamide; |
| 71 | 2-Cyclohexylamino-4-(2,6,6-trimethyl-4-oxo-4,5,6,7-tetrahydro-indol-1-yl)-benzamide; |
| 72 | 2-(butylamino)-4-(2,6,6-trimethyl-4-oxo-4,5,6,7-tetrahydroindol-1-yl)benzamide; |
| 73 | 4-Methyl-3-(2,6,6-trimethyl-4-oxo-4,5,6,7-tetrahydro-indol-1-yl)-benzamide; |
| 74 | 3-(3-thienyl)-4-(2,6,6-trimethyl-4-oxo-4,5,6,7-tetrahydro-1H-indol-1-yl)benzamide; |
| 75 | 2-methyl-4-(2,6,6-trimethyl-4-oxo-4,5,6,7-tetrahydro-1H-indol-1-yl)benzamide; |
| 76 | 2-[(3-ethynylphenyl)amino]-4-(2,6,6-trimethyl-4-oxo-4,5,6,7-tetrahydro-1H-indol-1- |
| | yl)benzamide; |
| 77 | 2-[(4-chlorophenyl)amino]-4-(2,6,6-trimethyl-4-oxo-4,5,6,7-tetrahydro-1H-indol-1- |
| | yl)benzamide; |
| 78 | 2-anilino-4-(2-methyl-4-oxo-4,5,6,7-tetrahydro-1H-indol-1-yl)benzamide; |
| 79 | 3-anilino-5-(2,6,6-trimethyl-4-oxo-4,5,6,7-tetrahydro-1H-indol-1-yl)pyridine-2- |
| | carboxamide; |
| 80 | 2-[(3,4,5-trimethoxyphenyl)amino]-4-(2,6,6-trimethyl-4-oxo-4,5,6,7-tetrahydro-1H- |
| | indol-1-yl)benzamide; |
| 81 | 2-pyridin-4-yl-4-(2,6,6-trimethyl-4-oxo-4,5,6,7-tetrahydro-1H-indol-1-yl)benzamide; |
| 82 | N-[2-(aminocarbonyl)-5-(2,6,6-trimethyl-4-oxo-4,5,6,7-tetrahydro-1H-indol-1-yl)phenyl]- |
| | L-valine; |
| 83 | 2-morpholin-4-yl-4-(2,6,6-trimethyl-4-oxo-4,5,6,7-tetrahydro-1H-indol-1-yl)benzamide; |
| 84 | 2-(1H-imidazol-1-yl)-4-(2,6,6-trimethyl-4-oxo-4,5,6,7-tetrahydro-1H-indol-1- |
| | yl)benzamide; |
| 85 | 4-(3-chloro-2,6,6-trimethyl-4-oxo-4,5,6,7-tetrahydro-1H-indol-1-yl)-2-[(3,4,5- |
| | trimethoxyphenyl)amino]benzamide; |
| 86 | 2-[(4-hydroxyphenyl)amino]-4-(2,6,6-trimethyl-4-oxo-4,5,6,7-tetrahydro-1H-indol-1- |
| | yl)benzamide; |
| 87 | 2-[(1-ethyl-1H-pyrazol-5-yl)amino]-4-(2,6,6-trimethyl-4-oxo-4,5,6,7-tetrahydro-1H-indol- |
| | 1-yl)benzamide; |
| 88 | 2-[(5-methylisoxazol-3-yl)amino]-4-(2,6,6-trimethyl-4-oxo-4,5,6,7-tetrahydro-1H-indol- |
| | 1-yl)benzamide; |
| 89 | 2-{[4-(aminocarbonyl)phenyl]amino}-4-(2-methyl-4-oxo-4,5,6,7-tetrahydro-1H-indol-1- |
| | yl)benzamide; |
| 90 | 4-(4-oxo-4,5,6,7-tetrahydro-1H-indol-1-yl)-2-[(3,4,5- |
| | trimethoxyphenyl)amino]benzamide; |
| 91 | 2-[(6-methoxypyridin-3-yl)amino]-4-(2,6,6-trimethyl-4-oxo-4,5,6,7-tetrahydro-1H-indol- |
| | 1-yl)benzamide; |
| 92 | 2-(allylamino)-4-(4-oxo-4,5,6,7-tetrahydro-1H-indol-1-yl)benzamide; |
| 93 | 4-(2,3-dimethyl-4-oxo-4,5,6,7-tetrahydro-1H-indol-1-yl)benzamide; |
| 94 | 3-bromo-4-(2,3-dimethyl-4-oxo-4,5,6,7-tetrahydro-1H-indol-1-yl)benzamide; |
| 95 | 2-(allylamino)-4-(2,3-dimethyl-4-oxo-4,5,6,7-tetrahydro-1H-indol-1-yl)benzamide; |
| 96 | 4-(2,3-dimethyl-4-oxo-4,5,6,7-tetrahydro-1H-indol-1-yl)-2-[(2- |
| | methoxyethyl)amino]benzamide; |
| 97 | 2-({3-[3-(dimethylamino)propoxy]-4-methoxyphenyl}amino)-4-(2,6,6-trimethyl-4-oxo- |
| | 4,5,6,7-tetrahydro-1H-indol-1-yl)benzamide; |
| 98 | 2-(morpholin-4-ylamino)-4-(2,6,6-trimethyl-4-oxo-4,5,6,7-tetrahydro-1H-indol-1- |
| | yl)benzamide; |
| 99 | 2-[(2-morpholin-4-ylethyl)amino]-4-(3,6,6-trimethyl-4-oxo-4,5,6,7-tetrahydro-1H-indol- |
| | 1-yl)benzamide; |
| 100 | 2-(pyridin-4-ylamino)-4-(3,6,6-trimethyl-4-oxo-4,5,6,7-tetrahydro-1H-indol-1- |
| | yl)benzamide; |
| 101 | 2-(acetylamino)-4-(3,6,6-trimethyl-4-oxo-4,5,6,7-tetrahydro-1H-indol-1-yl)benzamide; |
| 102 | 2-(4-methylpiperazin-1-yl)-4-(3,6,6-trimethyl-4-oxo-4,5,6,7-tetrahydro-1H-indol-1- |
| | yl)benzamide; |
| 103 | 2-[(cyclopropylmethyl)amino]-4-(3,6,6-trimethyl-4-oxo-4,5,6,7-tetrahydro-1H-indazol-1- |
| | yl)benzamide; |
| 104 | 2-[(methoxyacetyl)amino]-4-(3,6,6-trimethyl-4-oxo-4,5,6,7-tetrahydro-1H-indol-1- |
| | yl)benzamide; |
| 105 | 2-ethyl-4-(3,6,6-trimethyl-4-oxo-4,5,6,7-tetrahydro-1H-indazol-1-yl)benzamide; |
| 106 | 2-(butylthio)-4-(3,6,6-trimethyl-4-oxo-4,5,6,7-tetrahydro-1H-indazol-1-yl)benzamide; |
| 107 | 2-({3-[2-(dimethylamino)ethoxy]-4-methoxyphenyl}amino)-4-(3,6,6-trimethyl-4-oxo- |
| | 4,5,6,7-tetrahydro-1H-indol-1-yl)benzamide; |
| 108 | 2-(pyridin-4-ylthio)-4-(3,6,6-trimethyl-4-oxo-4,5,6,7-tetrahydro-1H-indol-1- |
| | yl)benzamide; |
| 109 | 2-{[1-phenylethyl]amino}-4-(3,6,6-trimethyl-4-oxo-4,5,6,7-tetrahydro-1H-indazol-1- |
| | yl)benzamide; |
| 110 | 4-[6,6-dimethyl-4-oxo-3-(trifluoromethyl)-4,5,6,7-tetrahydro-1H-indazol-1-yl]-2-[(3,4,5- |
| | trimethoxyphenyl)amino]benzamide; |
| 111 | 2-({2-[2-(dimethylamino)ethoxy]pyridin-4-yl}amino)-4-(3,6,6-trimethyl-4-oxo-4,5,6,7- |
| | tetrahydro-1H-indol-1-yl)benzamide; |
| 112 | 2-{[1-(N,N-dimethylglycyl)piperidin-4-yl]amino}-4-(3,6,6-trimethyl-4-oxo-4,5,6,7- |
| | tetrahydro-1H-indol-1-yl)benzamide; |
| 113 | 4-(6,6-dimethyl-4-oxo-3-phenyl-4,5,6,7-tetrahydro-1H-indol-1-yl)benzamide; |
| 114 | 2-[(2-{[2-(aminocarbonyl)-5-(3,6,6-trimethyl-4-oxo-4,5,6,7-tetrahydro-1H-indol-1- |
| | yl)phenyl]amino}ethyl)amino]-2-oxoethyl acetate; |
| 115 | 2-{[2-(glycoloylamino)ethyl]amino}-4-(3,6,6-trimethyl-4-oxo-4,5,6,7-tetrahydro-1H- |
| | indol-1-yl)benzamide; |
| 116 | 2-{[2-(methylsulfonyl)ethyl]amino}-4-(3,6,6-trimethyl-4-oxo-4,5,6,7-tetrahydro-1H- |
| | indazol-1-yl)benzamide; |
| 117 | 2-[(4-methoxyphenyl)amino]-4-(3,6,6-trimethyl-4-oxo-4,5,6,7-tetrahydro-1H-indazol-1- |
| | yl)benzamide; |
| 118 | 2-[(6-oxo-1,6-dihydropyridin-3-yl)amino]-4-(3,6,6-trimethyl-4-oxo-4,5,6,7-tetrahydro- |
| | 1H-indazol-1-yl)benzamide; |
| 119 | 2-(cyclopent-3-en-1-ylamino)-4-[3-(difluoromethyl)-6,6-dimethyl-4-oxo-4,5,6,7- |
| | tetrahydro-1H-indazol-1-yl]benzamide; |
| 120 | 2-(cyclobutylamino)-4-(3,6,6-trimethyl-4-oxo-4,5,6,7-tetrahydro-1H-indazol-1- |
| | yl)benzamide; |
| 121 | 2-[4-(2-Hydroxy-ethoxy)-cyclohexylamino]-4-(3,6,6-trimethyl-4-oxo-4,5,6,7-tetrahydro- |
| | indazol-1-yl)-benzamide; |
| 122 | 2-(4-Hydroxy-cyclohexylamino)-4-(3,6,6-trimethyl-4-oxo-4,5,6,7-tetrahydro-indazol-1- |
| | yl)-benzamide; |
| 123 | 4-[6,6-dimethyl-4-oxo-3-methyl-4,5,6,7-tetrahydro-1H-indazol-1-yl]-2-{[2-methoxy-1- |
| | (methoxymethyl)ethyl]amino}benzamide; |
| 124 | 2-{[3-hydroxy-1-(2-hydroxyethyl)propyl]amino}-4-(3,6,6-trimethyl-4-oxo-4,5,6,7- |
| | tetrahydro-1H-indazol-1-yl)benzamide; |
| 125 | 4-[6,6-dimethyl-4-oxo-3-(trifluoromethyl)-4,5,6,7-tetrahydro-1H-indazol-1-yl]-2-{[2- |
| | methoxy-1-(methoxymethyl)ethyl]amino}benzamide; |
| 126 | 2-{[3-(methylsulfinyl)phenyl]amino}-4-(3,6,6-trimethyl-4-oxo-4,5,6,7-tetrahydro-1H- |
| | indazol-1-yl)benzamide; |
| 127 | 4-[6,6-dimethyl-4-oxo-3-(trifluoromethyl)-4,5,6,7-tetrahydro-1H-indazol-1-yl]-2-{[1- |
| | (methylsulfonyl)piperidin-4-yl]amino}benzamide; |
| 128 | 4-(6,6-Dimethyl-4-oxo-3-trifluoromethyl-4,5,6,7-tetrahydro-indazol-1-yl)-2-[4-(2- |
| | hydroxy-ethoxy)-cyclohexylamino]-benzamide; |
| 129 | 2-{[1-(3-morpholin-4-ylpropanoyl)piperidin-4-yl]amino}-4-(3,6,6-trimethyl-4-oxo-4,5,6,7- |
| | tetrahydro-1H-indazol-1-yl)benzamide; |
| 130 | 2-[4-(2-Amino-ethoxy)-cyclohexylamino]-4-(6,6-dimethyl-4-oxo-3-trifluoromethyl- |
| | 4,5,6,7-tetrahydro-indazol-1-yl)-benzamide; |
| 131 | 2-[(1-glycylpiperidin-4-yl)amino]-4-(3,6,6-trimethyl-4-oxo-4,5,6,7-tetrahydro-1H- |
| | indazol-1-yl)benzamide; |
| 132 | 2-[4-(2-Amino-ethoxy)-cyclohexylamino]-4-(3,6,6-trimethyl-4-oxo-4,5,6,7-tetrahydro- |
| | indazol-1-yl)-benzamide; |
| 133 | 2-{[1-(methylsulfonyl)azetidin-3-yl]amino}-4-(3,6,6-trimethyl-4-oxo-4,5,6,7-tetrahydro- |
| | 1H-indazol-1-yl)benzamide |
| 134 | 2-{[3-(methylsulfonyl)propyl]amino}-4-(3,6,6-trimethyl-4-oxo-4,5,6,7-tetrahydro-1H- |
| | indazol-1-yl)benzamide; |
| 135 | 4-[6,6-dimethyl-4-oxo-3-(trifluoromethyl)-4,5,6,7-tetrahydro-1H-indazol-1-yl]-2-({2- |
| | [(methylsulfonyl)amino]ethyl}amino)benzamide; |
| 136 | 4-(3-but-3-en-1-yl-6,6-dimethyl-4-oxo-4,5,6,7-tetrahydro-1H-indazol-1-yl)benzamide; |
| 137 | 2-{[1-(methylsulfonyl)pyrrolidin-3-yl]amino}-4-(3,6,6-trimethyl-4-oxo-4,5,6,7-tetrahydro- |
| | 1H-indazol-1-yl)benzamide; |
| 138 | 2-({2-[(dimethylamino)sulfonyl]ethyl}amino)-4-[6,6-dimethyl-4-oxo-3-(trifluoromethyl)- |
| | 4,5,6,7-tetrahydro-1H-indazol-1-yl]benzamide; or |
| 139 | 4-[3-(2-Amino-ethyl)-6,6-dimethyl-4-oxo-4,5,6,7-tetrahydro-indazol-1-yl]-benzamide; |
| 140 | 4-(6,6-dimethyl-4-oxo-4,5,6,7-tetrahydro-1H-indazol-1-yl)benzoic acid; |
| 141 | 4-(6,6-dimethyl-4-oxo-4,5,6,7-tetrahydro-1H-indazol-1-yl)benzamide; |
| 142 | 4-(2,6,6-trimethyl-4-oxo-4,5,6,7-tetrahydro-1H-indol-1-yl)benzamide; |
| 143 | 4-[4-(methoxyimino)-6,6-dimethyl-4,5,6,7-tetrahydro-1H-indazol-1-yl]benzamide; |
| 144 | 4-[4-(hydroxyimino)-6,6-dimethyl-4,5,6,7-tetrahydro-1H-indazol-1-yl]benzamide; |
| 145 | 4-methyl-3-(2,6,6-trimethyl-4-oxo-4,5,6,7-tetrahydro-1H-indol-1-yl)benzamide; |
| 146 | 3-(2,6,6-trimethyl-4-oxo-4,5,6,7-tetrahydro-1H-indol-1-yl)benzamide; |
| 147 | 2-chloro-4-(2,6,6-trimethyl-4-oxo-4,5,6,7-tetrahydro-1H-indol-1-yl)benzoic acid; |
| 148 | 4-[4-(ethoxyimino)-6,6-dimethyl-4,5,6,7-tetrahydro-1H-indazol-1-yl]benzamide; |
| 149 | 4-[6,6-dimethyl-4-(phenoxyimino)-4,5,6,7-tetrahydro-1H-indazol-1-yl]benzamide; |
| 150 | 4-[4-(isobutoxyimino)-6,6-dimethyl-4,5,6,7-tetrahydro-1H-indazol-1-yl]benzamide; |
| 151 | 4-{4-[(allyloxy)imino]-6,6-dimethyl-4,5,6,7-tetrahydro-1H-indazol-1-yl}benzamide; |
| 152 | 4-{6,6-dimethyl-4-[(tetrahydro-2H-pyran-2-yloxy)imino]-4,5,6,7-tetrahydro-1H-indazol- |
| | 1-yl}benzamide; |
| 153 | 4-{4-[(benzyloxy)imino]-6,6-dimethyl-4,5,6,7-tetrahydro-1H-indazol-1-yl}benzamide; |
| 154 | 4-[4-(hydroxyimino)-2,6,6-trimethyl-4,5,6,7-tetrahydro-1H-indol-1-yl]benzamide; |
| 155 | 4-[(4-(methoxyimino)-2,6,6-trimethyl-4,5,6,7-tetrahydro-1H-indol-1-yl]benzamide; |
| 156 | 4-{4-[(allyloxy)imino]-2,6,6-trimethyl-4,5,6,7-tetrahydro-1H-indol-1-yl}benzamide; |
| 157 | 4-[4-(isobutoxyimino)-2,6,6-trimethyl-4,5,6,7-tetrahydro-1H-indol-1-yl]benzamide; |
| 158 | 4-{4-[(benzyloxy)imino]-2,6,6-trimethyl-4,5,6,7-tetrahydro-1H-indol-1-yl}benzamide; |
| 159 | 3-[4-(methoxyimino)-2,6,6-trimethyl-4,5,6,7-tetrahydro-1H-indol-1-yl]-4- |
| | methylbenzamide; |
| 160 | 3-[4-(hydroxyimino)-2,6,6-trimethyl-4,5,6,7-tetrahydro-1H-indol-1-yl]-4- |
| | methylbenzamide; |
| 161 | 4-[6-(1,3-benzodioxol-5-yl)-4-oxo-4,5,6,7-tetrahydro-1H-indazol-1-yl]benzoic acid; |
| 162 | 4-[6-(1,3-benzodioxol-5-yl)-4-oxo-4,5,6,7-tetrahydro-1H-indazol-1-yl]benzamide; |
| 163 | 4-[6-(1,3-benzodioxol-5-yl)-4-(hydroxyimino)-4,5,6,7-tetrahydro-1H-indazol-1- |
| | yl]benzamide; |
| 164 | 2-(trifluoromethyl)-4-(2,6,6-trimethyl-4-oxo-4,5,6,7-tetrahydro-1H-indol-1- |
| | yl)benzamide; |
| 165 | 2-methoxy-4-(2,6,6-trimethyl-4-oxo-4,5,6,7-tetrahydro-1H-indol-1-yl)benzamide; |
| 166 | 2-butoxy-4-(2,6,6-trimethyl-4-oxo-4,5,6,7-tetrahydro-1H-indol-1-yl)benzamide; |
| 167 | 2-(butylamino)-4-(2,6,6-trimethyl-4-oxo-4,5,6,7-tetrahydro-1H-indol-1-yl)benzamide; |
| 168 | 3-(2-ethoxyethoxy)-4-(2,6,6-trimethyl-4-oxo-4,5,6,7-tetrahydro-1H-indol-1- |
| | yl)benzamide; |
| 169 | 3-(2-methoxyethoxy)-4-(2,6,6-trimethyl-4-oxo-4,5,6,7-tetrahydro-1H-indol-1- |
| | yl)benzamide; |
| 170 | 2-ethoxy-4-(2,6,6-trimethyl-4-oxo-4,5,6,7-tetrahydro-1H-indol-1-yl)benzamide; |
| 171 | 2-(2-hydroxyethoxy)-4-(2,6,6-trimethyl-4-oxo-4,5,6,7-tetrahydro-1H-indol-1- |
| | yl)benzamide; |
| 172 | 2-[(2-methoxyethyl)amino]-4-(2,6,6-trimethyl-4-oxo-4,5,6,7-tetrahydro-1H-indol-1- |
| | yl)benzamide; |
| 173 | 2-(2-ethoxyethoxy)-4-(2,6,6-trimethyl-4-oxo-4,5,6,7-tetrahydro-1H-indol-1- |
| | yl)benzamide; |
| 174 | 4-(4-oxo-4,5,6,7-tetrahydro-1H-indol-1-yl)benzamide; |
| 175 | 3-[2-(dimethylamino)ethoxy]-4-(2,6,6-trimethyl-4-oxo-4,5,6,7-tetrahydro-1H-indol-1- |
| | yl)benzamide; |
| 176 | 3-(tetrahydrofuran-3-ylmethoxy)-4-(2,6,6-trimethyl-4-oxo-4,5,6,7-tetrahydro-1H-indol- |
| | 1-yl)benzamide; |
| 177 | 3-(4-fluorophenoxy)-4-(2,6,6-trimethyl-4-oxo-4,5,6,7-tetrahydro-1H-indol-1- |
| | yl)benzamide; |
| 178 | 2-{[2-(dimethylamino)ethyl]amino}-4-(2,6,6-trimethyl-4-oxo-4,5,6,7-tetrahydro-1H- |
| | indol-1-yl)benzamide; |
| 179 | 2-anilino-4-(2,6,6-trimethyl-4-oxo-4,5,6,7-tetrahydro-1H-indol-1-yl)benzamide; |
| 180 | 3-fluoro-4-(2,6,6-trimethyl-4-oxo-4,5,6,7-tetrahydro-1H-indol-1-yl)benzamide; |
| 181 | 4-(6,6-dimethyl-4-oxo-2-phenyl-4,5,6,7-tetrahydro-1H-indol-1-yl)benzamide; |
| 182 | 2-chloro-4-(2,6,6-trimethyl-4-oxo-4,5,6,7-tetrahydro-1H-indol-1-yl)benzamide; |
| 183 | 2-(benzylamino)-4-(2,6,6-trimethyl-4-oxo-4,5,6,7-tetrahydro-1H-indol-1-yl)benzamide; |
| 184 | 2-bromo-4-(2,6,6-trimethyl-4-oxo-4,5,6,7-tetrahydro-1H-indol-1-yl)benzamide; |
| 185 | 3-(2-hydroxyethoxy)-4-(2,6,6-trimethyl-4-oxo-4,5,6,7-tetrahydro-1H-indol-1- |
| | yl)benzamide; |
| 186 | 2-amino-4-(2,6,6-trimethyl-4-oxo-4,5,6,7-tetrahydro-1H-indol-1-yl)benzamide; |
| 187 | 3-(tert-butylthio)-4-(2,6,6-trimethyl-4-oxo-4,5,6,7-tetrahydro-1H-indol-1-yl)benzamide; |
| 188 | 3-(phenylthio)-4-(2,6,6-trimethyl-4-oxo-4,5,6,7-tetrahydro-1H-indol-1-yl)benzamide; |
| 189 | 3-(butylthio)-4-(2,6,6-trimethyl-4-oxo-4,5,6,7-tetrahydro-1H-indol-1-yl)benzamide; |
| 190 | 2-(dimethylamino)-4-(2,6,6-trimethyl-4-oxo-4,5,6,7-tetrahydro-1H-indol-1- |
| | yl)benzamide; |
| 191 | 3-methoxy-4-(2,6,6-trimethyl-4-oxo-4,5,6,7-tetrahydro-1H-indol-1-yl)benzamide; |
| 192 | 3-ethoxy-4-(2,6,6-trimethyl-4-oxo-4,5,6,7-tetrahydro-1H-indol-1-yl)benzamide; |
| 193 | 3-propoxy-4-(2,6,6-trimethyl-4-oxo-4,5,6,7-tetrahydro-1H-indol-1-yl)benzamide; |
| 194 | 3-(benzyloxy)-4-(2,6,6-trimethyl-4-oxo-4,5,6,7-tetrahydro-1H-indol-1-yl)benzamide; |
| 195 | 3-(2-morpholin-4-ylethoxy)-4-(2,6,6-trimethyl-4-oxo-4,5,6,7-tetrahydro-1H-indol-1- |
| | yl)benzamide; |
| 196 | 3-(pyridin-2-ylmethoxy)-4-(2,6,6-trimethyl-4-oxo-4,5,6,7-tetrahydro-1H-indol-1- |
| | yl)benzamide; |
| 197 | 3-(pyridin-4-ylmethoxy)-4-(2,6,6-trimethyl-4-oxo-4,5,6,7-tetrahydro-1H-indol-1- |
| | yl)benzamide; |
| 198 | 3-(2-isopropoxyethoxy)-4-(2,6,6-trimethyl-4-oxo-4,5,6,7-tetrahydro-1H-indol-1- |
| | yl)benzamide; |
| 199 | 3-(2-pyrrolidin-2-ylethoxy)-4-(2,6,6-trimethyl-4-oxo-4,5,6,7-tetrahydro-1H-indol-1- |
| | yl)benzamide; |
| 200 | 3-(tetrahydro-2H-pyran-2-ylmethoxy)-4-(2,6,6-trimethyl-4-oxo-4,5,6,7-tetrahydro-1H- |
| | indol-1-yl)benzamide; |
| 201 | 3-(tetrahydro-2H-pyran-4-yloxy)-4-(2,6,6-trimethyl-4-oxo-4,5,6,7-tetrahydro-1H-indol-1- |
| | yl)benzamide; |
| 202 | 3-(butylamino)-4-(2,6,6-trimethyl-4-oxo-4,5,6,7-tetrahydro-1H-indol-1-yl)benzamide; |
| 203 | 3-chloro-4-(2,6,6-trimethyl-4-oxo-4,5,6,7-tetrahydro-1H-indol-1-yl)benzamide; |
| 204 | 3-(1H-imidazol-4-ylmethoxy)-4-(2,6,6-trimethyl-4-oxo-4,5,6,7-tetrahydro-1H-indol-1- |
| | yl)benzamide; |
| 205 | 2-[(4-methoxyphenyl)amino]-4-(2,6,6-trimethyl-4-oxo-4,5,6,7-tetrahydro-1H-indol-1- |
| | yl)benzamide; |
| 206 | 3-butoxy-4-(2,6,6-trimethyl-4-oxo-4,5,6,7-tetrahydro-1H-indol-1-yl)benzoic acid; |
| 207 | 3-anilino-4-(2,6,6-trimethyl-4-oxo-4,5,6,7-tetrahydro-1H-indol-1-yl)benzamide; |
| 208 | 3-(benzylamino)-4-(2,6,6-trimethyl-4-oxo-4,5,6,7-tetrahydro-1H-indol-1-yl)benzamide; |
| 209 | 3-(3-hydroxypropoxy)-4-(2,6,6-trimethyl-4-oxo-4,5,6,7-tetrahydro-1H-indol-1- |
| | yl)benzamide; |
| 210 | 3-(3-hydroxybutoxy)-4-(2,6,6-trimethyl-4-oxo-4,5,6,7-tetrahydro-1H-indol-1- |
| | yl)benzamide; |
| 211 | 3-(2,3-dihydroxypropoxy)-4-(2,6,6-trimethyl-4-oxo-4,5,6,7-tetrahydro-1H-indol-1- |
| | yl)benzamide; |
| 212 | N-butyl-4-(6,6-dimethyl-4-oxo-4,5,6,7-tetrahydro-1H-indazol-1-yl)benzamide; |
| 213 | 3-(2-methylbutoxy)-4-(2,6,6-trimethyl-4-oxo-4,5,6,7-tetrahydro-1H-indol-1- |
| | yl)benzamide; |
| 214 | 3-(pentyloxy)-4-(2,6,6-trimethyl-4-oxo-4,5,6,7-tetrahydro-1H-indol-1-yl)benzamide; |
| 215 | 3-(3-methylbutoxy)-4-(2,6,6-trimethyl-4-oxo-4,5,6,7-tetrahydro-1H-indol-1- |
| | yl)benzamide; |
| 216 | 3-hydroxy-4-(2,6,6-trimethyl-4-oxo-4,5,6,7-tetrahydro-1H-indol-1-yl)benzamide; |
| 217 | 4-(6,6-dimethyl-4-oxo-4,5,6,7-tetrahydro-1H-indazol-1-yl)-N-phenylbenzamide; |
| 218 | 2-(cyclohexylamino)-4-(2,6,6-trimethyl-4-oxo-4,5,6,7-tetrahydro-1H-indol-1- |
| | yl)benzamide; |
| 219 | 3-(cyclohexyloxy)-4-(2,6,6-trimethyl-4-oxo-4,5,6,7-tetrahydro-1H-indol-1-yl)benzamide; |
| 220 | 3-(pent-4-en-1-yloxy)-4-(2,6,6-trimethyl-4-oxo-4,5,6,7-tetrahydro-1H-indol-1- |
| | yl)benzamide; |
| 221 | 2-(1,3-benzodioxol-5-ylamino)-4-(2,6,6-trimethyl-4-oxo-4,5,6,7-tetrahydro-1H-indol-1- |
| | yl)benzamide; |
| 222 | 2-(hexylamino)-4-(2,6,6-trimethyl-4-oxo-4,5,6,7-tetrahydro-1H-indol-1-yl)benzamide; |
| 223 | 3-butoxy-4-(2-methyl-4-oxo-4,5,6,7-tetrahydro-1H-indol-1-yl)benzamide; |
| 224 | 3-(4-hydroxybutoxy)-4-(2,6,6-trimethyl-4-oxo-4,5,6,7-tetrahydro-1H-indol-1- |
| | yl)benzamide; |
| 225 | 3-[(3-methyloxetan-3-yl)methoxy]-4-(2,6,6-trimethyl-4-oxo-4,5,6,7-tetrahydro-1H-indol- |
| | 1-yl)benzamide; |
| 226 | 3-(4-aminobutoxy)-4-(2,6,6-trimethyl-4-oxo-4,5,6,7-tetrahydro-1H-indol-1- |
| | yl)benzamide; |
| 227 | 3-(tetrahydrofuran-3-yloxy)-4-(2,6,6-trimethyl-4-oxo-4,5,6,7-tetrahydro-1H-indol-1- |
| | yl)benzamide; |
| 228 | 3-(tetrahydrofuran-2-ylmethoxy)-4-(2,6,6-trimethyl-4-oxo-4,5,6,7-tetrahydro-1H-indol- |
| | 1-yl)benzamide; |
| 229 | 3-[(1-ethylprop-2-en-1-yl)oxy]-4-(2,6,6-trimethyl-4-oxo-4,5,6,7-tetrahydro-1H-indol-1- |
| | yl)benzamide; |
| 230 | 2-bromo-4-(6,6-dimethyl-4-oxo-4,5,6,7-tetrahydro-1H-indazol-1-yl)benzamide; |
| 231 | 3-(2-thienylmethoxy)-4-(2,6,6-trimethyl-4-oxo-4,5,6,7-tetrahydro-1H-indol-1- |
| | yl)benzamide; |
| 232 | 4-(6,6-dimethyl-4-oxo-4,5,6,7-tetrahydro-1H-indol-1-yl)benzamide; |
| 233 | 2-[(methylsulfonyl)amino]-4-(2,6,6-trimethyl-4-oxo-4,5,6,7-tetrahydro-1H-indol-1- |
| | yl)benzamide; |
| 234 | 2-(acetylamino)-4-(2,6,6-trimethyl-4-oxo-4,5,6,7-tetrahydro-1H-indol-1-yl)benzamide; |
| 235 | 2-[(aminocarbonyl)amino]-4-(2,6,6-trimethyl-4-oxo-4,5,6,7-tetrahydro-1H-indol-1- |
| | yl)benzamide; |
| 236 | 2-(benzoylamino)-4-(2,6,6-trimethyl-4-oxo-4,5,6,7-tetrahydro-1H-indol-1-yl)benzamide; |
| 237 | 2-(butyrylamino)-4-(2,6,6-trimethyl-4-oxo-4,5,6,7-tetrahydro-1H-indol-1-yl)benzamide; |
| 238 | 3-ethoxy-5-(2,6,6-trimethyl-4-oxo-4,5,6,7-tetrahydro-1H-indol-1-yl)pyridine-2- |
| | carboxamide; |
| 239 | 2-(pyridin-3-ylamino)-4-(2,6,6-trimethyl-4-oxo-4,5,6,7-tetrahydro-1H-indol-1- |
| | yl)benzamide; |
| 240 | 2-(pyridin-2-ylamino)-4-(2,6,6-trimethyl-4-oxo-4,5,6,7-tetrahydro-1H-indol-1-yl)benzoic |
| | acid; |
| 241 | 2-[(3-ethoxyphenyl)amino]-4-(2,6,6-trimethyl-4-oxo-4,5,6,7-tetrahydro-1H-indol-1- |
| | yl)benzamide; |
| 242 | 2-[(3-methoxyphenyl)amino]-4-(2,6,6-trimethyl-4-oxo-4,5,6,7-tetrahydro-1H-indol-1- |
| | yl)benzamide; |
| 243 | 3-butoxy-4-(6,6-dimethyl-4-oxo-4,5,6,7-tetrahydro-1H-indazol-1-yl)benzamide; |
| 244 | 3-butoxy-4-(6,6-dimethyl-4-oxo-4,5,6,7-tetrahydro-1H-indazol-1-yl)-N′- |
| | hydroxybenzenecarboximidamide; |
| 245 | N′-hydroxy-3-methyl-4-(2,6,6-trimethyl-4-oxo-4,5,6,7-tetrahydro-1H-indol-1- |
| | yl)benzenecarboximidamide; |
| 246 | 3-methyl-4-(2,6,6-trimethyl-4-oxo-4,5,6,7-tetrahydro-1H-indol-1-yl)benzamide; |
| 247 | 2-chloro-N-hydroxy-4-(2,6,6-trimethyl-4-oxo-4,5,6,7-tetrahydro-1H-indol-1- |
| | yl)benzenecarboximidamide; |
| 248 | 2,3-difluoro-N-hydroxy-4-(2,6,6-trimethyl-4-oxo-4,5,6,7-tetrahydro-1H-indol-1- |
| | yl)benzenecarboximidamide; |
| 249 | 8-(2,6,6-trimethyl-4-oxo-4,5,6,7-tetrahydro-1H-indol-1-yl)-2,3-dihydro-1,4- |
| | benzodioxine-5-carboxamide; |
| 250 | 2-pentyl-4-(2,6,6-trimethyl-4-oxo-4,5,6,7-tetrahydro-1H-indol-1-yl)benzamide; |
| 251 | 3-pentyl-4-(2,6,6-trimethyl-4-oxo-4,5,6,7-tetrahydro-1H-indol-1-yl)benzamide; |
| 252 | 2-[(4-butoxyphenyl)amino]-4-(2,6,6-trimethyl-4-oxo-4,5,6,7-tetrahydro-1H-indol-1- |
| | yl)benzamide; |
| 253 | 2-anilino-N′-hydroxy-4-(2,6,6-trimethyl-4-oxo-4,5,6,7-tetrahydro-1H-indol-1- |
| | yl)benzenecarboximidamide; |
| 254 | 2-{[4-(trifluoromethoxy)phenyl]amino}-4-(2,6,6-trimethyl-4-oxo-4,5,6,7-tetrahydro-1H- |
| | indol-1-yl)benzamide; |
| 255 | 2-[(2-chloro-4-fluorophenyl)amino]-4-(2,6,6-trimethyl-4-oxo-4,5,6,7-tetrahydro-1H- |
| | indol-1-yl)benzamide; |
| 256 | 2-[(2-chloro-4-methylphenyl)amino]-4-(2,6,6-trimethyl-4-oxo-4,5,6,7-tetrahydro-1H- |
| | indol-1-yl)benzamide; |
| 257 | 2-[(2-chloro-4-methoxyphenyl)amino]-4-(2,6,6-trimethyl-4-oxo-4,5,6,7-tetrahydro-1H- |
| | indol-1-yl)benzamide; |
| 258 | 2-[(3,4-dimethoxyphenyl)amino]-4-(2,6,6-trimethyl-4-oxo-4,5,6,7-tetrahydro-1H-indol- |
| | 1-yl)benzamide; |
| 259 | 2-[(3-fluoro-4-methoxyphenyl)amino]-4-(2,6,6-trimethyl-4-oxo-4,5,6,7-tetrahydro-1H- |
| | indol-1-yl)benzamide; |
| 260 | 2-[(3,5-dimethoxyphenyl)amino]-4-(2,6,6-trimethyl-4-oxo-4,5,6,7-tetrahydro-1H-indol- |
| | 1-yl)benzamide; |
| 261 | 2-[(2,5-dimethoxyphenyl)amino]-4-(2,6,6-trimethyl-4-oxo-4,5,6,7-tetrahydro-1H-indol- |
| | 1-yl)benzamide; |
| 262 | 2-[(4-ethoxyphenyl)amino]-4-(2,6,6-trimethyl-4-oxo-4,5,6,7-tetrahydro-1H-indol-1- |
| | yl)benzamide; |
| 263 | 2-[(4-fluorophenyl)amino]-4-(2,6,6-trimethyl-4-oxo-4,5,6,7-tetrahydro-1H-indol-1- |
| | yl)benzamide; |
| 264 | 2-phenoxy-4-(2,6,6-trimethyl-4-oxo-4,5,6,7-tetrahydro-1H-indol-1-yl)benzamide; |
| 265 | 2-(phenylthio)-4-(2,6,6-trimethyl-4-oxo-4,5,6,7-tetrahydro-1H-indol-1-yl)benzamide; |
| 266 | 2-(cyclopropylamino)-4-(2,6,6-trimethyl-4-oxo-4,5,6,7-tetrahydro-1H-indol-1- |
| | yl)benzamide; |
| 267 | 2-(prop-2-yn-1-ylamino)-4-(2,6,6-trimethyl-4-oxo-4,5,6,7-tetrahydro-1H-indol-1- |
| | yl)benzamide; |
| 268 | 3-(propylthio)-4-(2,6,6-trimethyl-4-oxo-4,5,6,7-tetrahydro-1H-indol-1-yl)benzamide; |
| 269 | N-hydroxy-3-(propylthio)-4-(2,6,6-trimethyl-4-oxo-4,5,6,7-tetrahydro-1H-indol-1- |
| | yl)benzenecarboximidamide; |
| 270 | N-[2-(aminocarbonyl)-5-(2,6,6-trimethyl-4-oxo-4,5,6,7-tetrahydro-1H-indol-1-yl)phenyl]- |
| | L-phenylalanine; |
| 271 | 3-butoxy-4-[4-(hydroxyimino)-6,6-dimethyl-4,5,6,7-tetrahydro-1H-indazol-1- |
| | yl]benzamide; |
| 272 | 2-(diethylamino)ethyl 3-butoxy-4-(2,6,6-trimethyl-4-oxo-4,5,6,7-tetrahydro-1H-indol-1- |
| | yl)benzoate; |
| 273 | 2-[(2-chloro-3,4,5-trimethoxyphenyl)amino]-4-(3-chloro-2,6,6-trimethyl-4-oxo-4,5,6,7- |
| | tetrahydro-1H-indol-1-yl)benzamide; |
| 274 | 4-(6,6-dimethyl-4-oxo-4,5,6,7-tetrahydro-1H-indol-1-yl)-2-[(3,4,5- |
| | trimethoxyphenyl)amino]benzamide; |
| 275 | 4-(2-methyl-4-oxo-4,5,6,7-tetrahydro-1H-indol-1-yl)-2-[(3,4,5- |
| | trimethoxyphenyl)amino]benzamide; |
| 276 | 2-[(3,4,5-trifluorophenyl)amino]-4-(2,6,6-trimethyl-4-oxo-4,5,6,7-tetrahydro-1H-indol-1- |
| | yl)benzamide; |
| 277 | 2-[(4-oxocyclohexyl)amino]-4-(2,6,6-trimethyl-4-oxo-4,5,6,7-tetrahydro-1H-indol-1- |
| | yl)benzamide; |
| 278 | 2-[(4-methylphenyl)amino]-4-(2,6,6-trimethyl-4-oxo-4,5,6,7-tetrahydro-1H-indol-1- |
| | yl)benzamide; |
| 279 | 2-(2,3-dihydro-1H-inden-4-ylamino)-4-(2,6,6-trimethyl-4-oxo-4,5,6,7-tetrahydro-1H- |
| | indol-1-yl)benzamide; |
| 280 | 2-[(2-propylphenyl)amino]-4-(2,6,6-trimethyl-4-oxo-4,5,6,7-tetrahydro-1H-indol-1- |
| | yl)benzamide; |
| 281 | 2-[(2-isopropylphenyl)amino]-4-(2,6,6-trimethyl-4-oxo-4,5,6,7-tetrahydro-1H-indol-1- |
| | yl)benzamide; |
| 282 | 2-(3-hydroxyprop-1-yn-1-yl)-4-(2,6,6-trimethyl-4-oxo-4,5,6,7-tetrahydro-1H-indol-1- |
| | yl)benzamide; |
| 283 | 2-[(2-chloro-3,4,5-trimethoxyphenyl)amino]-4-(2,6,6-trimethyl-4-oxo-4,5,6,7-tetrahydro- |
| | 1H-indol-1-yl)benzamide; |
| 284 | 4-(4-oxo-2-phenyl-4,5,6,7-tetrahydro-1H-indol-1-yl)benzamide; |
| 285 | 2-[(2,4,5-trifluorophenyl)amino]-4-(2,6,6-trimethyl-4-oxo-4,5,6,7-tetrahydro-1H-indol-1- |
| | yl)benzamide; |
| 286 | 2-[(3,4,5-trimethoxybenzyl)amino]-4-(2,6,6-trimethyl-4-oxo-4,5,6,7-tetrahydro-1H-indol- |
| | 1-yl)benzamide; |
| 287 | 2-[(2-fluorophenyl)amino]-4-(2,6,6-trimethyl-4-oxo-4,5,6,7-tetrahydro-1H-indol-1- |
| | yl)benzamide; |
| 288 | 2-[(3-fluorophenyl)amino]-4-(2,6,6-trimethyl-4-oxo-4,5,6,7-tetrahydro-1H-indol-1- |
| | yl)benzamide; |
| 289 | 2-[(4-fluoro-3-methylphenyl)amino]-4-(2,6,6-trimethyl-4-oxo-4,5,6,7-tetrahydro-1H- |
| | indol-1-yl)benzamide; |
| 290 | 2-[(3-hydroxy-4-methoxyphenyl)amino]-4-(2,6,6-trimethyl-4-oxo-4,5,6,7-tetrahydro-1H- |
| | indol-1-yl)benzamide; |
| 291 | 2-[(2-fluoro-4-methoxyphenyl)amino]-4-(2,6,6-trimethyl-4-oxo-4,5,6,7-tetrahydro-1H- |
| | indol-1-yl)benzamide; |
| 292 | 2-(tetrahydro-2H-pyran-4-ylamino)-4-(2,6,6-trimethyl-4-oxo-4,5,6,7-tetrahydro-1H- |
| | indol-1-yl)benzamide; |
| 293 | 2-[(2,4-difluorophenyl)amino]-4-(2,6,6-trimethyl-4-oxo-4,5,6,7-tetrahydro-1H-indol-1- |
| | yl)benzamide; |
| 294 | 2-[(3,4-difluorophenyl)amino]-4-(2,6,6-trimethyl-4-oxo-4,5,6,7-tetrahydro-1H-indol-1- |
| | yl)benzamide; |
| 295 | 2-anilino-4-{5-[(dimethylamino)methyl]-2,6,6-trimethyl-4-oxo-4,5,6,7-tetrahydro-1H- |
| | indol-1-yl}benzamide; |
| 296 | 4-[4-(hydroxyimino)-2-phenyl-4,5,6,7-tetrahydro-1H-indol-1-yl]benzamide; |
| 297 | 2-{[4-(benzyloxy)phenyl]amino}-4-(2,6,6-trimethyl-4-oxo-4,5,6,7-tetrahydro-1H-indol-1- |
| | yl)benzamide; |
| 298 | 2-[(tetrahydrofuran-2-ylmethyl)amino]-4-(2,6,6-trimethyl-4-oxo-4,5,6,7-tetrahydro-1H- |
| | indol-1-yl)benzamide; |
| 299 | 2-[(1,3-dioxolan-2-ylmethyl)amino]-4-(2,6,6-trimethyl-4-oxo-4,5,6,7-tetrahydro-1H- |
| | indol-1-yl)benzamide; |
| 300 | 2-[(2-methoxy-1-methylethyl)amino]-4-(2,6,6-trimethyl-4-oxo-4,5,6,7-tetrahydro-1H- |
| | indol-1-yl)benzamide; |
| 301 | 2-{[2-(2-hydroxyethoxy)ethyl]amino}-4-(2,6,6-trimethyl-4-oxo-4,5,6,7-tetrahydro-1H- |
| | indol-1-yl)benzamide; |
| 302 | 2-[(4-hydroxybutyl)amino]-4-(2-methyl-4-oxo-4,5,6,7-tetrahydro-1H-indol-1- |
| | yl)benzamide; |
| 303 | 4-(2-methyl-4-oxo-4,5,6,7-tetrahydro-1H-indol-1-yl)-2-[(3-morpholin-4- |
| | ylpropyl)amino]benzamide; |
| 304 | 2-[(pyridin-2-ylmethyl)amino]-4-(2,6,6-trimethyl-4-oxo-4,5,6,7-tetrahydro-1H-indol-1- |
| | yl)benzamide; |
| 305 | 2-{[2-(diethylamino)-4-ethoxyphenyl]amino}-4-(2,6,6-trimethyl-4-oxo-4,5,6,7- |
| | tetrahydro-1H-indol-1-yl)benzamide; |
| 306 | 2-{[4-(difluoromethoxy)phenyl]amino}-4-(2,6,6-trimethyl-4-oxo-4,5,6,7-tetrahydro-1H- |
| | indol-1-yl)benzamide; |
| 307 | 2-[(3,4-dimethoxybenzyl)amino]-4-(2,6,6-trimethyl-4-oxo-4,5,6,7-tetrahydro-1H-indol-1- |
| | yl)benzamide; |
| 308 | 2-[(3-hydroxypropyl)amino]-4-(2,6,6-trimethyl-4-oxo-4,5,6,7-tetrahydro-1H-indol-1- |
| | yl)benzamide; |
| 309 | 2-(allylamino)-4-(2,6,6-trimethyl-4-oxo-4,5,6,7-tetrahydro-1H-indol-1-yl)benzamide; |
| 310 | 4-[4-(hydroxyimino)-2,6,6-trimethyl-4,5,6,7-tetrahydro-1H-indol-1-yl]-2-[(3,4,5- |
| | trimethoxyphenyl)amino]benzamide; |
| 311 | 2-[(3-methoxypropyl)amino]-4-(2,6,6-trimethyl-4-oxo-4,5,6,7-tetrahydro-1H-indol-1- |
| | yl)benzamide; |
| 312 | 2-(pyridin-4-ylamino)-4-(2,6,6-trimethyl-4-oxo-4,5,6,7-tetrahydro-1H-indol-1- |
| | yl)benzamide; |
| 313 | 2-{[3-(1H-imidazol-1-yl)propyl]amino}-4-(2,6,6-trimethyl-4-oxo-4,5,6,7-tetrahydro-1H- |
| | indol-1-yl)benzamide; |
| 314 | 2-{[2-(1H-imidazol-1-yl)ethyl]amino}-4-(2,6,6-trimethyl-4-oxo-4,5,6,7-tetrahydro-1H- |
| | indol-1-yl)benzamide; |
| 315 | 2-(isoxazol-3-ylamino)-4-(2,6,6-trimethyl-4-oxo-4,5,6,7-tetrahydro-1H-indol-1- |
| | yl)benzamide; |
| 316 | 2-[(pyridin-3-ylmethyl)amino]-4-(2,6,6-trimethyl-4-oxo-4,5,6,7-tetrahydro-1H-indol-1- |
| | yl)benzamide; |
| 317 | 4-(2,6,6-trimethyl-4-oxo-4,5,6,7-tetrahydro-1H-indol-1-yl)phthalamide; |
| 318 | 3-bromo-N′-hydroxy-4-(4-oxo-4,5,6,7-tetrahydro-1H-indol-1- |
| | yl)benzenecarboximidamide; |
| 319 | 4-(2,6,6-trimethyl-4-oxo-4,5,6,7-tetrahydro-1H-indol-1-yl)phthalic acid; |
| 320 | 2-[(10-aminodecyl)amino]-4-(2,6,6-trimethyl-4-oxo-4,5,6,7-tetrahydro-1H-indol-1- |
| | yl)benzamide; |
| 321 | 2-(allylamino)-4-(2-methyl-4-oxo-4,5,6,7-tetrahydro-1H-indol-1-yl)benzamide; |
| 322 | 2-(2,3-dihydro-1H-inden-1-ylamino)-4-(4-oxo-4,5,6,7-tetrahydro-1H-indol-1- |
| | yl)benzamide; |
| 323 | 2-(cyclopropylamino)-4-(2,3-dimethyl-4-oxo-4,5,6,7-tetrahydro-1H-indol-1- |
| | yl)benzamide; |
| 324 | 2-[(2-methoxyethyl)amino]-4-(2-methyl-4-oxo-4,5,6,7-tetrahydro-1H-indol-1- |
| | yl)benzamide; |
| 325 | 3-[(cyclopropylmethyl)amino]-4-(4-oxo-4,5,6,7-tetrahydro-1H-indol-1-yl)benzamide; |
| 326 | 2-{[3-(3-hydroxypropoxy)-4-methoxyphenyl]amino}-4-(2,6,6-trimethyl-4-oxo-4,5,6,7- |
| | tetrahydro-1H-indol-1-yl)benzamide; |
| 327 | 2-(allylamino)-4-(3-methyl-4-oxo-4,5,6,7-tetrahydro-1H-indol-1-yl)benzamide; |
| 328 | 2-(cyclopropylamino)-4-(3-methyl-4-oxo-4,5,6,7-tetrahydro-1H-indol-1-yl)benzamide; |
| 329 | 2-[(2-methoxyethyl)amino]-4-(3-methyl-4-oxo-4,5,6,7-tetrahydro-1H-indol-1- |
| | yl)benzamide; |
| 330 | 4-(2-methyl-4-oxo-4,5,6,7-tetrahydro-1H-indol-1-yl)benzamide; |
| 331 | 4-(3-ethyl-2-methyl-4-oxo-4,5,6,7-tetrahydro-1H-indol-1-yl)benzamide; |
| 332 | 4-(3-methyl-4-oxo-4,5,6,7-tetrahydro-1H-indol-1-yl)benzamide; |
| 333 | 2-[(2-{2-[2-(2-aminoethoxy)ethoxy]ethoxy}ethyl)amino]-4-(2,6,6-trimethyl-4-oxo- |
| | 4,5,6,7-tetrahydro-1H-indol-1-yl)benzamide; |
| 334 | 2-[(4-hydroxycyclohexyl)amino]-4-(2-methyl-4-oxo-4,5,6,7-tetrahydro-1H-indol-1- |
| | yl)benzamide; |
| 335 | 4-(3,6,6-trimethyl-4-oxo-4,5,6,7-tetrahydro-1H-indol-1-yl)benzamide; |
| 336 | 2-anilino-4-(3-methyl-4-oxo-4,5,6,7-tetrahydro-1H-indol-1-yl)benzamide; |
| 337 | 2-[(4-hydroxycyclohexyl)amino]-4-(3-methyl-4-oxo-4,5,6,7-tetrahydro-1H-indol-1- |
| | yl)benzamide; |
| 338 | 4-(2,3-dimethyl-4-oxo-4,5,6,7-tetrahydro-1H-indol-1-yl)-2-[(4- |
| | hydroxycyclohexyl)amino]benzamide; |
| 339 | 2-anilino-4-(2,3-dimethyl-4-oxo-4,5,6,7-tetrahydro-1H-indol-1-yl)benzamide; |
| 340 | 4-(3-methyl-4-oxo-4,5,6,7-tetrahydro-1H-indol-1-yl)-2-[(3,4,5- |
| | trimethoxyphenyl)amino]benzamide; |
| 341 | 2-{[2-(dimethylamino)ethyl]amino}-4-(3,6,6-trimethyl-4-oxo-4,5,6,7-tetrahydro-1H- |
| | indol-1-yl)benzamide; |
| 342 | 2-{[2-(dimethylamino)ethyl]amino}-4-(3-methyl-4-oxo-4,5,6,7-tetrahydro-1H-indol-1- |
| | yl)benzamide; |
| 343 | 2-(piperidin-4-ylamino)-4-(2,6,6-trimethyl-4-oxo-4,5,6,7-tetrahydro-1H-indol-1- |
| | yl)benzamide; |
| 344 | 4-(3,6,6-trimethyl-4-oxo-4,5,6,7-tetrahydro-1H-indazol-1-yl)benzamide; |
| 345 | 2-bromo-5-(4-oxo-4,5,6,7-tetrahydro-1H-indol-1-yl)benzamide; |
| 346 | 2-[(4-hydroxycyclohexyl)amino]-4-(3,6,6-trimethyl-4-oxo-4,5,6,7-tetrahydro-1H-indol-1- |
| | yl)benzamide; |
| 347 | 2-[(1-methylpiperidin-4-yl)amino]-4-(2,6,6-trimethyl-4-oxo-4,5,6,7-tetrahydro-1H-indol- |
| | 1-yl)benzamide; |
| 348 | (4-{[2-(aminocarbonyl)-5-(2,6,6-trimethyl-4-oxo-4,5,6,7-tetrahydro-1H-indol-1- |
| | yl)phenyl]amino}piperidin-1-yl)acetic acid; |
| 349 | 2-[(2-methoxyethyl)amino]-4-(3,6,6-trimethyl-4-oxo-4,5,6,7-tetrahydro-1H-indol-1- |
| | yl)benzamide; |
| 350 | 2-[(pyridin-4-ylmethyl)amino]-4-(3,6,6-trimethyl-4-oxo-4,5,6,7-tetrahydro-1H-indol-1- |
| | yl)benzamide; |
| 351 | 2-(tetrahydro-2H-pyran-4-ylamino)-4-(3,6,6-trimethyl-4-oxo-4,5,6,7-tetrahydro-1H- |
| | indol-1-yl)benzamide; |
| 352 | 2-[(3,4,5-trimethoxyphenyl)amino]-4-(3,6,6-trimethyl-4-oxo-4,5,6,7-tetrahydro-1H- |
| | indol-1-yl)benzamide; |
| 353 | 4-(3-isobutyl-6,6-dimethyl-4-oxo-4,5,6,7-tetrahydro-1H-indazol-1-yl)benzamide; |
| 354 | 2-amino-4-(3,6,6-trimethyl-4-oxo-4,5,6,7-tetrahydro-1H-indol-1-yl)benzamide; |
| 355 | 2-(allylamino)-4-(3,6,6-trimethyl-4-oxo-4,5,6,7-tetrahydro-1H-indol-1-yl)benzamide; |
| 356 | 2-[(1-methylpiperidin-4-yl)amino]-4-(3,6,6-trimethyl-4-oxo-4,5,6,7-tetrahydro-1H-indol- |
| | 1-yl)benzamide; |
| 357 | 2-(piperidin-4-ylamino)-4-(3,6,6-trimethyl-4-oxo-4,5,6,7-tetrahydro-1H-indol-1- |
| | yl)benzamide; |
| 358 | 3-butoxy-4-(3,6,6-trimethyl-4-oxo-4,5,6,7-tetrahydro-1H-indol-1-yl)benzamide; |
| 359 | 2-(2,3-dihydro-1H-inden-1-ylamino)-4-(3,6,6-trimethyl-4-oxo-4,5,6,7-tetrahydro-1H- |
| | indol-1-yl)benzamide; |
| 360 | 2-[(4-oxocyclohexyl)amino]-4-(3,6,6-trimethyl-4-oxo-4,5,6,7-tetrahydro-1H-indol-1- |
| | yl)benzamide; |
| 361 | 2-[(aminocarbonyl)amino]-4-(3,6,6-trimethyl-4-oxo-4,5,6,7-tetrahydro-1H-indol-1- |
| | yl)benzamide; |
| 362 | 2-(tetrahydro-2H-pyran-4-ylamino)-4-(3,6,6-trimethyl-4-oxo-4,5,6,7-tetrahydro-1H- |
| | indazol-1-yl)benzamide; |
| 363 | 2-bromo-4-(3,6,6-trimethyl-4-oxo-4,5,6,7-tetrahydro-1H-indazol-1-yl)benzamide; |
| 364 | 2-[(3-morpholin-4-ylpropyl)amino]-4-(3,6,6-trimethyl-4-oxo-4,5,6,7-tetrahydro-1H- |
| | indazol-1-yl)benzamide; |
| 365 | 2-[(tetrahydrofuran-2-ylmethyl)amino]-4-(3,6,6-trimethyl-4-oxo-4,5,6,7-tetrahydro-1H- |
| | indazol-1-yl)benzamide; |
| 366 | 2-{[4-(2-morpholin-4-ylethoxy)cyclohexyl]amino}-4-(3,6,6-trimethyl-4-oxo-4,5,6,7- |
| | tetrahydro-1H-indol-1-yl)benzamide; |
| 367 | 2-[(8-methyl-8-azabicyclo[3.2.1]oct-3-yl)amino]-4-(3,6,6-trimethyl-4-oxo-4,5,6,7- |
| | tetrahydro-1H-indol-1-yl)benzamide; |
| 368 | 4-(3-ethyl-6,6-dimethyl-4-oxo-4,5,6,7-tetrahydro-1H-indazol-1-yl)benzamide; |
| 369 | 2-(cyclohexylamino)-4-(3,6,6-trimethyl-4-oxo-4,5,6,7-tetrahydro-1H-indol-1- |
| | yl)benzamide; |
| 370 | 2-[(1-methylpiperidin-4-yl)amino]-4-(3,6,6-trimethyl-4-oxo-4,5,6,7-tetrahydro-1H- |
| | indazol-1-yl)benzamide; |
| 371 | 2-(cyclopropylamino)-4-(3,6,6-trimethyl-4-oxo-4,5,6,7-tetrahydro-1H-indazol-1- |
| | yl)benzamide; |
| 372 | 2-(cyclopropylamino)-4-(3,6,6-trimethyl-4-oxo-4,5,6,7-tetrahydro-1H-indol-1- |
| | yl)benzamide; |
| 373 | 2-[(cyclopropylmethyl)amino]-4-(3,6,6-trimethyl-4-oxo-4,5,6,7-tetrahydro-1H-indol-1- |
| | yl)benzamide; |
| 374 | 2-[(3,4-dimethoxyphenyl)amino]-4-(3,6,6-trimethyl-4-oxo-4,5,6,7-tetrahydro-1H-indol- |
| | 1-yl)benzamide; |
| 375 | 2-[(3,4-dimethoxyphenyl)amino]-4-(3,6,6-trimethyl-4-oxo-4,5,6,7-tetrahydro-1H- |
| | indazol-1-yl)benzamide; |
| 376 | 2-[(3,4,5-trimethoxyphenyl)amino]-4-(3,6,6-trimethyl-4-oxo-4,5,6,7-tetrahydro-1H- |
| | indazol-1-yl)benzamide; |
| 377 | 2-({3-[3-(dimethylamino)propoxy]-4-methoxyphenyl}amino)-4-(3,6,6-trimethyl-4-oxo- |
| | 4,5,6,7-tetrahydro-1H-indol-1-yl)benzamide; |
| 378 | 2-[(4-oxocyclohexyl)amino]-4-(3,6,6-trimethyl-4-oxo-4,5,6,7-tetrahydro-1H-indazol-1- |
| | yl)benzamide; |
| 379 | 2-{[2-(aminocarbonyl)-5-(3,6,6-trimethyl-4-oxo-4,5,6,7-tetrahydro-1H-indol-1- |
| | yl)phenyl]amino}-2-oxoethyl acetate; |
| 380 | 2-{[3-(1H-imidazol-1-yl)propyl]amino}-4-(3,6,6-trimethyl-4-oxo-4,5,6,7-tetrahydro-1H- |
| | indazol-1-yl)benzamide; |
| 381 | 4-{[2-(aminocarbonyl)-5-(3,6,6-trimethyl-4-oxo-4,5,6,7-tetrahydro-1H-indol-1- |
| | yl)phenyl]amino}cyclohexyl glycinate; |
| 382 | 2-[(2-aminoethyl)amino]-4-(3,6,6-trimethyl-4-oxo-4,5,6,7-tetrahydro-1H-indol-1- |
| | yl)benzamide; |
| 383 | 4-{[2-(aminocarbonyl)-5-(3,6,6-trimethyl-4-oxo-4,5,6,7-tetrahydro-1H-indol-1- |
| | yl)phenyl]amino}cyclohexyl acetate; |
| 384 | 4-(3-ethyl-4-oxo-4,5,6,7-tetrahydro-1H-indazol-1-yl)-2-[(tetrahydrofuran-2- |
| | ylmethyl)amino]benzamide; |
| 385 | 2-[(1,4-dioxan-2-ylmethyl)amino]-4-(3,6,6-trimethyl-4-oxo-4,5,6,7-tetrahydro-1H- |
| | indazol-1-yl)benzamide; |
| 386 | 2-[(2-furylmethyl)amino]-4-(3,6,6-trimethyl-4-oxo-4,5,6,7-tetrahydro-1H-indazol-1- |
| | yl)benzamide; |
| 387 | 2-[(2-furylmethyl)amino]-4-(3,6,6-trimethyl-4-oxo-4,5,6,7-tetrahydro-1H-indol-1- |
| | yl)benzamide; |
| 388 | 2-[(2-piperazin-1-ylethyl)amino]-4-(3,6,6-trimethyl-4-oxo-4,5,6,7-tetrahydro-1H-indazol- |
| | 1-yl)benzamide; |
| 389 | 2-[(2-piperazin-1-ylethyl)amino]-4-(3,6,6-trimethyl-4-oxo-4,5,6,7-tetrahydro-1H-indol-1- |
| | yl)benzamide; |
| 390 | 2-({3-[2-(dimethylamino)ethoxy]-4-methoxyphenyl}amino)-4-(3,6,6-trimethyl-4-oxo- |
| | 4,5,6,7-tetrahydro-1H-indazol-1-yl)benzamide; |
| 391 | 4-[6,6-dimethyl-4-oxo-3-(trifluoromethyl)-4,5,6,7-tetrahydro-1H-indazol-1- |
| | yl]benzamide; |
| 392 | 2-(allylamino)-4-(3,6,6-trimethyl-4-oxo-4,5,6,7-tetrahydro-1H-indazol-1-yl)benzamide; |
| 393 | 4-{[2-(aminocarbonyl)-5-(3,6,6-trimethyl-4-oxo-4,5,6,7-tetrahydro-1H-indazol-1- |
| | yl)phenyl]amino}cyclohexyl glycinate; |
| 394 | 2-[(4-hydroxycyclohexyl)amino]-4-(3,6,6-trimethyl-4-oxo-4,5,6,7-tetrahydro-1H-indazol- |
| | 1-yl)benzamide; |
| 395 | 4-[4-oxo-3-(trifluoromethyl)-4,5,6,7-tetrahydro-1H-indazol-1-yl]benzamide; |
| 396 | 4-(3-ethyl-4-oxo-4,5,6,7-tetrahydro-1H-indazol-1-yl)-N′-hydroxy-2-[(tetrahydrofuran-2- |
| | ylmethyl)amino]benzenecarboximidamide; |
| 397 | 2-{[2-(1-methylpyrrolidin-2-yl)ethyl]amino}-4-(3,6,6-trimethyl-4-oxo-4,5,6,7-tetrahydro- |
| | 1H-indazol-1-yl)benzamide; |
| 398 | 2-{[2-(1-methylpyrrolidin-2-yl)ethyl]amino}-4-(3,6,6-trimethyl-4-oxo-4,5,6,7-tetrahydro- |
| | 1H-indol-1-yl)benzamide; |
| 399 | 2-{[2-(dimethylamino)ethyl]amino}-4-(3,6,6-trimethyl-4-oxo-4,5,6,7-tetrahydro-1H- |
| | indazol-1-yl)benzamide; |
| 400 | 4-{[2-(aminocarbonyl)-5-(3,6,6-trimethyl-4-oxo-4,5,6,7-tetrahydro-1H-indol-1- |
| | yl)phenyl]amino}cyclohexyl N,N- |
| 401 | dimethylglycinate; |
| 402 | 3-chloro-4-(3,6,6-trimethyl-4-oxo-4,5,6,7-tetrahydro-1H-indazol-1-yl)benzamide; |
| 403 | 4-(3,6,6-trimethyl-4-oxo-4,5,6,7-tetrahydro-1H-indazol-1-yl)benzoic acid; |
| 404 | 2-(pyridin-3-ylamino)-4-(3,6,6-trimethyl-4-oxo-4,5,6,7-tetrahydro-1H-indazol-1- |
| | yl)benzamide; |
| 405 | 2-(pyridin-4-ylthio)-4-(3,6,6-trimethyl-4-oxo-4,5,6,7-tetrahydro-1H-indazol-1- |
| | yl)benzamide; |
| 406 | 2-[(1-glycylpiperidin-4-yl)amino]-4-(3,6,6-trimethyl-4-oxo-4,5,6,7-tetrahydro-1H-indol-1- |
| | yl)benzamide; |
| 407 | N-hydroxy-4-(3,6,6-trimethyl-4-oxo-4,5,6,7-tetrahydro-1H-indazol-1-yl)benzamide; |
| 408 | N-(2-methoxyethyl)-4-(3,6,6-trimethyl-4-oxo-4,5,6,7-tetrahydro-1H-indazol-1- |
| | yl)benzamide; |
| 409 | 2-{[1-phenylethyl]amino}-4-(3,6,6-trimethyl-4-oxo-4,5,6,7-tetrahydro-1H-indol-1- |
| | yl)benzamide; |
| 410 | 2-[(1-ethylpiperidin-3-yl)amino]-4-(3,6,6-trimethyl-4-oxo-4,5,6,7-tetrahydro-1H-indol-1- |
| | yl)benzamide; |
| 411 | 2-{[1-phenylethyl]amino}-4-(3,6,6-trimethyl-4-oxo-4,5,6,7-tetrahydro-1H-indol-1- |
| | yl)benzamide; |
| 412 | 2-{[1-phenylethyl]amino}-4-(3,6,6-trimethyl-4-oxo-4,5,6,7-tetrahydro-1H-indazol-1- |
| | yl)benzamide; |
| 413 | 2-[(4-methoxycyclohexyl)amino]-4-(3,6,6-trimethyl-4-oxo-4,5,6,7-tetrahydro-1H-indol- |
| | 1-yl)benzamide; |
| 414 | 2-{[3-(2-oxopyrrolidin-1-yl)propyl]amino}-4-(3,6,6-trimethyl-4-oxo-4,5,6,7-tetrahydro- |
| | 1H-indazol-1-yl)benzamide; |
| 415 | 2-({3-[3-(dimethylamino)propoxy]-4-methoxyphenyl}amino)-4-[6,6-dimethyl-4-oxo-3- |
| | (trifluoromethyl)-4,5,6,7-tetrahydro-1H-indazol-1-yl]benzamide; |
| 416 | 2-(2,1,3-benzothiadiazol-4-ylamino)-4-(3,6,6-trimethyl-4-oxo-4,5,6,7-tetrahydro-1H- |
| | indazol-1-yl)benzamide; |
| 417 | 2-[(3-chlorophenyl)amino]-4-(3,6,6-trimethyl-4-oxo-4,5,6,7-tetrahydro-1H-indazol-1- |
| | yl)benzamide; |
| 418 | 2-[(3-methoxyphenyl)amino]-4-(3,6,6-trimethyl-4-oxo-4,5,6,7-tetrahydro-1H-indazol-1- |
| | yl)benzamide; |
| 419 | 2-({2-[2-(dimethylamino)ethoxy]pyridin-4-yl}amino)-4-(3,6,6-trimethyl-4-oxo-4,5,6,7- |
| | tetrahydro-1H-indazol-1-yl)benzamide; |
| 420 | 4-[6,6-dimethyl-4-oxo-3-(trifluoromethyl)-4,5,6,7-tetrahydro-1H-indazol-1-yl]-2-[(4- |
| | hydroxycyclohexyl)amino]benzamide; |
| 421 | 4-[6,6-dimethyl-4-oxo-3-(trifluoromethyl)-4,5,6,7-tetrahydro-1H-indazol-1-yl]-2- |
| | (tetrahydro-2H-pyran-4-ylamino)benzamide; |
| 422 | 2-{[2-hydroxy-2,3-dihydro-1H-inden-1-yl]amino}-4-(3,6,6-trimethyl-4-oxo-4,5,6,7- |
| | tetrahydro-1H-indazol-1-yl)benzamide; |
| 423 | 3-{[2-(aminocarbonyl)-5-(3,6,6-trimethyl-4-oxo-4,5,6,7-tetrahydro-1H-indazol-1- |
| | yl)phenyl]amino}benzoic acid; |
| 424 | 4-{[2-(aminocarbonyl)-5-(3,6,6-trimethyl-4-oxo-4,5,6,7-tetrahydro-1H-indazol-1- |
| | yl)phenyl]amino}cyclohexyl N,N-dimethylglycinate; |
| 425 | 2-[(1-oxidotetrahydro-2H-thiopyran-4-yl)oxy]-4-(3,6,6-trimethyl-4-oxo-4,5,6,7- |
| | tetrahydro-1H-indazol-1-yl)benzamide; |
| 426 | 2-[(1,1-dioxidotetrahydro-2H-thiopyran-4-yl)oxy]-4-(3,6,6-trimethyl-4-oxo-4,5,6,7- |
| | tetrahydro-1H-indazol-1-yl)benzamide; |
| 427 | 4-(3-methyl-4-oxo-5,6,7,8-tetrahydrocyclohepta[b]pyrrol-1(4H)-yl)-2-(tetrahydro-2H- |
| | pyran-4-ylamino)benzamide; |
| 428 | 2-{[2-(methylthio)ethyl]amino}-4-(3,6,6-trimethyl-4-oxo-4,5,6,7-tetrahydro-1H-indazol- |
| | 1-yl)benzamide; |
| 429 | 2-({3-[2-(dimethylamino)ethoxy]-4-methoxyphenyl}amino)-4-[6,6-dimethyl-4-oxo-3- |
| | (trifluoromethyl)-4,5,6,7-tetrahydro-1H-indazol-1-yl]benzamide; |
| 430 | 2-({2-[2-(dimethylamino)ethoxy]pyridin-4-yl}amino)-4-[6,6-dimethyl-4-oxo-3- |
| | (trifluoromethyl)-4,5,6,7-tetrahydro-1H-indazol-1-yl]benzamide; |
| 431 | 4-{[2-(aminocarbonyl)-5-(3,6,6-trimethyl-4-oxo-4,5,6,7-tetrahydro-1H-indazol-1- |
| | yl)phenyl]amino}cyclohexyl (acetyloxy)acetate; |
| 432 | 2-(tetrahydro-2H-thiopyran-4-ylamino)-4-(3,6,6-trimethyl-4-oxo-4,5,6,7-tetrahydro-1H- |
| | indazol-1-yl)benzamide; |
| 433 | 2-[(1-oxidotetrahydro-2H-thiopyran-4-yl)amino]-4-(3,6,6-trimethyl-4-oxo-4,5,6,7- |
| | tetrahydro-1H-indazol-1-yl)benzamide; |
| 434 | 2-[(1,1-dioxidotetrahydro-2H-thiopyran-4-yl)amino]-4-(3,6,6-trimethyl-4-oxo-4,5,6,7- |
| | tetrahydro-1H-indazol-1-yl)benzamide; |
| 435 | 2-{[3-(aminocarbonyl)phenyl]amino}-4-(3,6,6-trimethyl-4-oxo-4,5,6,7-tetrahydro-1H- |
| | indazol-1-yl)benzamide; |
| 436 | 2-(cyclopent-3-en-1-ylamino)-4-(3,6,6-trimethyl-4-oxo-4,5,6,7-tetrahydro-1H-indazol-1- |
| | yl)benzamide; |
| 437 | 2-[(2,2,2-trifluoroethyl)amino]-4-(3,6,6-trimethyl-4-oxo-4,5,6,7-tetrahydro-1H-indazol-1- |
| | yl)benzamide; |
| 438 | 2-{[3,4-dihydroxycyclopentyl]amino}-4-(3,6,6-trimethyl-4-oxo-4,5,6,7-tetrahydro-1H- |
| | indazol-1-yl)benzamide; |
| 439 | 2-[(6-methoxypyridin-3-yl)amino]-4-(3,6,6-trimethyl-4-oxo-4,5,6,7-tetrahydro-1H- |
| | indazol-1-yl)benzamide; |
| 440 | 2-[(1-methyl-6-oxo-1,6-dihydropyridin-3-yl)amino]-4-(3,6,6-trimethyl-4-oxo-4,5,6,7- |
| | tetrahydro-1H-indazol-1-yl)benzamide; |
| 441 | 4-{[2-(aminocarbonyl)-5-(3,6,6-trimethyl-4-oxo-4,5,6,7-tetrahydro-1H-indazol-1- |
| | yl)phenyl]amino}cyclohexyl methoxyacetate; |
| 442 | 2-({1-[3-(dimethylamino)propyl]-6-oxo-1,6-dihydropyridin-3-yl}amino)-4-(3,6,6- |
| | trimethyl-4-oxo-4,5,6,7-tetrahydro-1H-indazol-1-yl)benzamide; |
| 443 | 2-{[2-(2-oxopyrrolidin-1-yl)ethyl]amino}-4-(3,6,6-trimethyl-4-oxo-4,5,6,7-tetrahydro-1H- |
| | indazol-1-yl)benzamide; |
| 444 | 2-{[3-(trifluoromethoxy)phenyl]amino}-4-(3,6,6-trimethyl-4-oxo-4,5,6,7-tetrahydro-1H- |
| | indazol-1-yl)benzamide; |
| 445 | 4-[3-(difluoromethyl)-6,6-dimethyl-4-oxo-4,5,6,7-tetrahydro-1H-indazol-1-yl]-2- |
| | (tetrahydro-2H-pyran-4-ylamino)benzamide; |
| 446 | 4-[3-(difluoromethyl)-6,6-dimethyl-4-oxo-4,5,6,7-tetrahydro-1H-indazol-1-yl]-2-[(4- |
| | hydroxycyclohexyl)amino]benzamide; |
| 447 | 4-[6,6-dimethyl-4-oxo-3-(trifluoromethyl)-4,5,6,7-tetrahydro-1H-indazol-1-yl]-2- |
| | (tetrahydro-2H-thiopyran-4-ylamino)benzamide; |
| 448 | 4-[3-(difluoromethyl)-6,6-dimethyl-4-oxo-4,5,6,7-tetrahydro-1H-indazol-1-yl]-2- |
| | (tetrahydro-2H-thiopyran-4-ylamino)benzamide; |
| 449 | 2-[(2-methoxy-1-methylethyl)amino]-4-(3,6,6-trimethyl-4-oxo-4,5,6,7-tetrahydro-1H- |
| | indazol-1-yl)benzamide; |
| 450 | N-(2-aminophenyl)-3-butoxy-4-(2,6,6-trimethyl-4-oxo-4,5,6,7-tetrahydro-1H-indol-1- |
| | yl)benzamide; |
| 451 | 2-{[6-oxopiperidin-3-yl]amino}-4-(3,6,6-trimethyl-4-oxo-4,5,6,7-tetrahydro-1H-indazol- |
| | 1-yl)benzamide; |
| 452 | 4-{[2-(aminocarbonyl)-5-(3,6,6-trimethyl-4-oxo-4,5,6,7-tetrahydro-1H-indazol-1- |
| | yl)phenyl]amino}cyclohexyl L-alaninate; |
| 453 | 4-{[2-(aminocarbonyl)-5-(3,6,6-trimethyl-4-oxo-4,5,6,7-tetrahydro-1H-indazol-1- |
| | yl)phenyl]amino}cyclohexyl D-alaninate; |
| 454 | 4-{[2-(aminocarbonyl)-5-(3,6,6-trimethyl-4-oxo-4,5,6,7-tetrahydro-1H-indol-1- |
| | yl)phenyl]amino}cyclohexyl L-alaninate; |
| 455 | 4-{[2-(aminocarbonyl)-5-(3,6,6-trimethyl-4-oxo-4,5,6,7-tetrahydro-1H-indol-1- |
| | yl)phenyl]amino}cyclohexyl D-alaninate; |
| 456 | 1-[4-(aminocarbonyl)-3-(tetrahydro-2H-pyran-4-ylamino)phenyl]-6,6-dimethyl-4-oxo- |
| | 4,5,6,7-tetrahydro-1H-indazole-3-carboxylic acid; |
| 457 | 5-bromo-2-[(tetrahydrofuran-2-ylmethyl)amino]-4-(3,6,6-trimethyl-4-oxo-4,5,6,7- |
| | tetrahydro-1H-indazol-1-yl)benzamide; |
| 458 | 2-[(3-hydroxycyclohexyl)amino]-4-(3,6,6-trimethyl-4-oxo-4,5,6,7-tetrahydro-1H-indazol- |
| | 1-yl)benzamide; |
| 459 | 4-{[2-(aminocarbonyl)-5-(3,6,6-trimethyl-4-oxo-4,5,6,7-tetrahydro-1H-indazol-1- |
| | yl)phenyl]amino}cyclohexanecarboxylic acid; |
| 460 | 4-({2-(aminocarbonyl)-5-[6,6-dimethyl-4-oxo-3-(trifluoromethyl)-4,5,6,7-tetrahydro-1H- |
| | indazol-1-yl]phenyl}amino)cyclohexyl glycinate; |
| 461 | 4-{[2-(aminocarbonyl)-5-(3,6,6-trimethyl-4-oxo-4,5,6,7-tetrahydro-1H-indol-1- |
| | yl)phenyl]amino}cyclohexyl L-valinate; |
| 462 | 2-[(2,6-dihydroxytetrahydro-2H-pyran-4-yl)amino]-4-(3,6,6-trimethyl-4-oxo-4,5,6,7- |
| | tetrahydro-1H-indazol-1-yl)benzamide; |
| 463 | 2-(cyclopent-3-en-1-ylamino)-4-[6,6-dimethyl-4-oxo-3-(trifluoromethyl)-4,5,6,7- |
| | tetrahydro-1H-indazol-1-yl]benzamide; |
| 464 | 4-[3-(difluoromethyl)-6,6-dimethyl-4-oxo-4,5,6,7-tetrahydro-1H-indazol-1-yl]-2-[(4- |
| | oxocyclohexyl)amino]benzamide; |
| 465 | 4-[6,6-dimethyl-4-oxo-3-(trifluoromethyl)-4,5,6,7-tetrahydro-1H-indazol-1-yl]-2-[(4- |
| | oxocyclohexyl)amino]benzamide; |
| 466 | 4-[6,6-dimethyl-4-oxo-3-(trifluoromethyl)-4,5,6,7-tetrahydro-1H-indazol-1-yl]-2-[(1- |
| | oxidotetrahydro-2H-thiopyran-4-yl)amino]benzamide; |
| 467 | 4-[3-(difluoromethyl)-6,6-dimethyl-4-oxo-4,5,6,7-tetrahydro-1H-indazol-1-yl]-2-[(1- |
| | oxidotetrahydro-2H-thiopyran-4-yl)amino]benzamide; |
| 468 | 2-(cyclohex-3-en-1-ylamino)-4-(3,6,6-trimethyl-4-oxo-4,5,6,7-tetrahydro-1H-indazol-1- |
| | yl)benzamide; |
| 469 | 2-{[3,4-dihydroxycyclohexyl]amino}-4-(3,6,6-trimethyl-4-oxo-4,5,6,7-tetrahydro-1H- |
| | indazol-1-yl)benzamide; |
| 470 | 2-{[4-(trifluoromethoxy)phenyl]amino}-4-(3,6,6-trimethyl-4-oxo-4,5,6,7-tetrahydro-1H- |
| | indazol-1-yl)benzamide; |
| 471 | 4-({2-(aminocarbonyl)-5-[3-(difluoromethyl)-6,6-dimethyl-4-oxo-4,5,6,7-tetrahydro-1H- |
| | indazol-1-yl]phenyl}amino)cyclohexyl glycinate; |
| 472 | 4-[6,6-dimethyl-4-oxo-3-(trifluoromethyl)-4,5,6,7-tetrahydro-1H-indazol-1-yl]-2-[(3- |
| | ethynylphenyl)amino]benzamide; |
| 473 | 4-(6,6-dimethyl-4-oxo-4,5,6,7-tetrahydro-1H-indazol-1-yl)-2-(tetrahydro-2H-pyran-4- |
| | ylamino)benzamide; |
| 474 | 4-[6,6-dimethyl-4-oxo-3-(trifluoromethyl)-4,5,6,7-tetrahydro-1H-indazol-1-yl]-2-{[3-(2- |
| | oxopyrrolidin-1-yl)propyl]amino}benzamide; |
| 475 | 4-(trifluoromethyl)-3-(2,6,6-trimethyl-4-oxo-4,5,6,7-tetrahydro-1H-indol-1- |
| | yl)benzamide; |
| 476 | 2-{[3-(methylthio)phenyl]amino}-4-(3,6,6-trimethyl-4-oxo-4,5,6,7-tetrahydro-1H- |
| | indazol-1-yl)benzamide; |
| 477 | 4-(trifluoromethyl)-3-(3,6,6-trimethyl-4-oxo-4,5,6,7-tetrahydro-1H-indazol-1- |
| | yl)benzamide; |
| 478 | 3-(3-methyl-4-oxo-4,5,6,7-tetrahydro-1H-indazol-1-yl)-4-(trifluoromethyl)benzamide; |
| 479 | 2-[6,6-dimethyl-4-oxo-3-(trifluoromethyl)-4,5,6,7-tetrahydro-1H-indazol-1-yl]-4-[(4- |
| | hydroxycyclohexyl)amino]benzamide; |
| 480 | 4-[6,6-dimethyl-4-oxo-3-(trifluoromethyl)-4,5,6,7-tetrahydro-1H-indazol-1-yl]-2- |
| | (piperidin-4-ylamino)benzamide; |
| 481 | 2-[(1-acetylpiperidin-4-yl)amino]-4-[6,6-dimethyl-4-oxo-3-(trifluoromethyl)-4,5,6,7- |
| | tetrahydro-1H-indazol-1-yl]benzamide; |
| 482 | 2-[(1-benzylpiperidin-4-yl)amino]-4-[6,6-dimethyl-4-oxo-3-(trifluoromethyl)-4,5,6,7- |
| | tetrahydro-1H-indazol-1-yl]benzamide; |
| 483 | 4-({2-(aminocarbonyl)-5-[6,6-dimethyl-4-oxo-3-(trifluoromethyl)-4,5,6,7-tetrahydro-1H- |
| | indazol-1-yl]phenyl}amino)-N,N-dimethylpiperidine-1-carboxamide; |
| 484 | 2-bromo-4-[6,6-dimethyl-4-oxo-3-(trifluoromethyl)-4,5,6,7-tetrahydro-1H-indazol-1-yl]- |
| | N′-hydroxybenzenecarboximidamide; |
| 485 | 2-[(1-benzylpyrrolidin-3-yl)amino]-4-[6,6-dimethyl-4-oxo-3-(trifluoromethyl)-4,5,6,7- |
| | tetrahydro-1H-indazol-1-yl]benzamide; |
| 486 | 4-[6,6-dimethyl-4-oxo-3-(trifluoromethyl)-4,5,6,7-tetrahydro-1H-indazol-1-yl]-2-[(1- |
| | phenylpiperidin-4-yl)amino]benzamide; |
| 487 | ({[1-[4-(aminocarbonyl)-3-(tetrahydro-2H-pyran-4-ylamino)phenyl]-6,6-dimethyl-3- |
| | (trifluoromethyl)-1,5,6,7-tetrahydro-4H-indazol-4-ylidene]amino}oxy)acetic acid; |
| 488 | 4-[6,6-dimethyl-4-oxo-3-(trifluoromethyl)-4,5,6,7-tetrahydro-1H-indazol-1-yl]-2-{[2- |
| | hydroxy-1,1-bis(hydroxymethyl)ethyl]amino}benzamide; |
| 489 | 4-[6,6-dimethyl-4-oxo-3-(trifluoromethyl)-4,5,6,7-tetrahydro-1H-indazol-1-yl]-2- |
| | [tetrahydrofuran-3-ylamino]benzamide; |
| 490 | 4-({2-(aminocarbonyl)-5-[6,6-dimethyl-4-oxo-3-(trifluoromethyl)-4,5,6,7-tetrahydro-1H- |
| | indazol-1-yl]phenyl}amino)cyclohexyl L-alaninate; methanesulfonate |
| 491 | 4-({2-(aminocarbonyl)-5-[6,6-dimethyl-4-oxo-3-(trifluoromethyl)-4,5,6,7-tetrahydro-1H- |
| | indazol-1-yl]phenyl}amino)cyclohexyl L-valinate methanesulfonate; |
| 492 | 2-[allyl(4-hydroxycyclohexyl)amino]-4-[6,6-dimethyl-4-oxo-3-(trifluoromethyl)-4,5,6,7- |
| | tetrahydro-1H-indazol-1-yl]benzamide; |
| 493 | 2-Amino-propionic acid 4-[2-carbamoyl-5-(6,6-dimethyl-4-oxo-3-trifluoromethyl-4,5,6,7- |
| | tetrahydro-indazol-1-yl)-phenylamino]-cyclohexyl ester; |
| 494 | 4-[6,6-dimethyl-4-oxo-3-(trifluoromethyl)-4,5,6,7-tetrahydro-1H-indazol-1-yl]-2-(4- |
| | pyridin-2-ylpiperazin-1-yl)benzamide; |
| 495 | 2-[(2,3-dihydroxypropyl)(4-hydroxycyclohexyl)amino]-4-[6,6-dimethyl-4-oxo-3- |
| | (trifluoromethyl)-4,5,6,7-tetrahydro-1H-indazol-1-yl]benzamide; |
| 496 | 4-[6,6-dimethyl-4-oxo-3-(trifluoromethyl)-4,5,6,7-tetrahydro-1H-indazol-1-yl]-2-[(4- |
| | hydroxycyclohexyl)(2-oxoethyl)amino]benzamide; |
| 497 | 2-[(2,3-dihydroxypropyl)amino]-4-[6,6-dimethyl-4-oxo-3-(trifluoromethyl)-4,5,6,7- |
| | tetrahydro-1H-indazol-1-yl]benzamide; |
| 498 | 2-[(2,3-dihydroxypropyl)amino]-4-[6,6-dimethyl-4-oxo-3-(trifluoromethyl)-4,5,6,7- |
| | tetrahydro-1H-indazol-1-yl]benzoic acid; |
| 499 | 2-(acetoxymethyl)-2-(2-carbamoyl-5-(6,6-dimethyl-4-oxo-3-(trifluoromethyl)-4,5,6,7- |
| | tetrahydro-1H-indazol-1-yl)phenylamino)propane-1,3-diyl diacetate; |
| 500 | 2-({2-(aminocarbonyl)-5-[6,6-dimethyl-4-oxo-3-(trifluoromethyl)-4,5,6,7-tetrahydro-1H- |
| | indazol-1-yl]phenyl}amino)-2-(hydroxymethyl)propane-1,3-diyl diacetate; |
| 501 | 2-({2-(aminocarbonyl)-5-[6,6-dimethyl-4-oxo-3-(trifluoromethyl)-4,5,6,7-tetrahydro-1H- |
| | indazol-1-yl]phenyl}amino)-3-hydroxy-2-(hydroxymethyl)propyl acetate; |
| 502 | 4-[6,6-dimethyl-4-oxo-3-(trifluoromethyl)-4,5,6,7-tetrahydro-1H-indazol-1-yl]-2-[(4- |
| | hydroxycyclohexyl)(2-hydroxyethyl)amino]benzamide; |
| 503 | 2-({4-[2,2,2-trifluoro-1-hydroxy-1-(trifluoromethyl)ethyl]phenyl}amino)-4-(3,6,6- |
| | trimethyl-4-oxo-4,5,6,7-tetrahydro-1H-indazol-1-yl)benzamide; |
| 504 | 2-({1-[3-(4-methylpiperazin-1-yl)propanoyl]piperidin-4-yl}amino)-4-(3,6,6-trimethyl-4- |
| | oxo-4,5,6,7-tetrahydro-1H-indazol-1-yl)benzamide; |
| 505 | 2-[(1-isonicotinoylpiperidin-4-yl)amino]-4-(3,6,6-trimethyl-4-oxo-4,5,6,7-tetrahydro-1H- |
| | indazol-1-yl)benzamide; |
| 506 | 2-{[1-(pyridin-3-ylcarbonyl)piperidin-4-yl]amino}-4-(3,6,6-trimethyl-4-oxo-4,5,6,7- |
| | tetrahydro-1H-indazol-1-yl)benzamide; |
| 507 | 2-(1-azabicyclo[2.2.2]oct-3-ylamino)-4-(3,6,6-trimethyl-4-oxo-4,5,6,7-tetrahydro-1H- |
| | indazol-1-yl)benzamide; |
| 508 | 2-{[1-(methylsulfonyl)pyrrolidin-3-yl]amino}-4-(3,6,6-trimethyl-4-oxo-4,5,6,7-tetrahydro- |
| | 1H-indazol-1-yl)benzamide; |
| 509 | 2-[(1-beta-alanylpiperidin-4-yl)amino]-4-(3,6,6-trimethyl-4-oxo-4,5,6,7-tetrahydro-1H- |
| | indazol-1-yl)benzamide; |
| 510 | 2-[(1-prolylpiperidin-4-yl)amino]-4-(3,6,6-trimethyl-4-oxo-4,5,6,7-tetrahydro-1H-indazol- |
| | 1-yl)benzamide; |
| 511 | 2-{[1-acetylpyrrolidin-3-yl]amino}-4-(3,6,6-trimethyl-4-oxo-4,5,6,7-tetrahydro-1H- |
| | indazol-1-yl)benzamide; |
| 512 | 3-{[2-(aminocarbonyl)-5-(3,6,6-trimethyl-4-oxo-4,5,6,7-tetrahydro-1H-indazol-1- |
| | yl)phenyl]amino}-N,N-dimethylpyrrolidine-1-carboxamide; |
| 513 | [[2-Carbamoyl-5-(6,6-dimethyl-4-oxo-3-trifluoromethyl-4,5,6,7-tetrahydro-indazol-1-yl)- |
| | phenyl]-(4-hydroxy-cyclohexyl)-amino]-acetic acid; |
| 514 | 2-{[4-(hydroxymethyl)phenyl]amino}-4-(3,6,6-trimethyl-4-oxo-4,5,6,7-tetrahydro-1H- |
| | indazol-1-yl)benzamide; |
| 515 | 4-[6,6-dimethyl-4-oxo-3-(trifluoromethyl)-4,5,6,7-tetrahydro-1H-indazol-1-yl]-2-{[3- |
| | hydroxycyclopentyl]amino}benzamide; |
| 516 | 2-{[tetrahydrofuran-2-ylmethyl]amino}-4-(3,6,6-trimethyl-4-oxo-4,5,6,7-tetrahydro-1H- |
| | indazol-1-yl)benzamide; |
| 517 | 2-{[4-(allyloxy)cyclohexyl]amino}-4-[6,6-dimethyl-4-oxo-3-(trifluoromethyl)-4,5,6,7- |
| | tetrahydro-1H-indazol-1-yl]benzamide; |
| 518 | 2-[(1-isonicotinoylazetidin-3-yl)amino]-4-(3,6,6-trimethyl-4-oxo-4,5,6,7-tetrahydro-1H- |
| | indazol-1-yl)benzamide; |
| 519 | 2-{[1-(pyridin-3-ylcarbonyl)azetidin-3-yl]amino}-4-(3,6,6-trimethyl-4-oxo-4,5,6,7- |
| | tetrahydro-1H-indazol-1-yl)benzamide; |
| 520 | 2-{[1-(3-morpholin-4-ylpropanoyl)azetidin-3-yl]amino}-4-(3,6,6-trimethyl-4-oxo-4,5,6,7- |
| | tetrahydro-1H-indazol-1-yl)benzamide; |
| 521 | 2-({1-[3-(4-methylpiperazin-1-yl)propanoyl]azetidin-3-yl}amino)-4-(3,6,6-trimethyl-4- |
| | oxo-4,5,6,7-tetrahydro-1H-indazol-1-yl)benzamide; |
| 522 | 2-{[3-(methylthio)propyl]amino}-4-(3,6,6-trimethyl-4-oxo-4,5,6,7-tetrahydro-1H-indazol- |
| | 1-yl)benzamide; |
| 523 | 2-[6,6-dimethyl-4-oxo-3-(trifluoromethyl)-4,5,6,7-tetrahydro-1H-indazol-1-yl]-4- |
| | [tetrahydrofuran-3-ylamino]benzamide; |
| 524 | 2-{[2-methoxy-1-methylethyl]amino}-4-(3,6,6-trimethyl-4-oxo-4,5,6,7-tetrahydro-1H- |
| | indazol-1-yl)benzamide; |
| 525 | 2-(tetrahydro-2H-pyran-2-ylamino)-4-(3,6,6-trimethyl-4-oxo-4,5,6,7-tetrahydro-1H- |
| | indol-1-yl)benzamide; |
| 526 | 2-[[4-(allyloxy)cyclohexyl](3,5-dimethoxybenzyl)amino]-4-[6,6-dimethyl-4-oxo-3- |
| | (trifluoromethyl)-4,5,6,7-tetrahydro-1H-indazol-1-yl]benzamide; |
| 527 | 2-[[4-(2,3-dihydroxypropoxy)cyclohexyl](3,5-dimethoxybenzyl)amino]-4-[6,6-dimethyl- |
| | 4-oxo-3-(trifluoromethyl)-4,5,6,7-tetrahydro-1H-indazol-1-yl]benzamide; |
| 528 | 4-{3-[(benzyloxy)methyl]-6,6-dimethyl-4-oxo-4,5,6,7-tetrahydro-1H-indazol-1- |
| | yl}benzamide; |
| 529 | 2-({2-[(dimethylamino)sulfonyl]ethyl}amino)-4-(3,6,6-trimethyl-4-oxo-4,5,6,7- |
| | tetrahydro-1H-indazol-1-yl)benzamide; |
| 530 | 2-[pyrrolidin-3-ylamino]-4-(3,6,6-trimethyl-4-oxo-4,5,6,7-tetrahydro-1H-indazol-1- |
| | yl)benzamide hydrochloride; |
| 531 | 2-{[1-acetylpyrrolidin-3-yl]amino}-4-(3,6,6-trimethyl-4-oxo-4,5,6,7-tetrahydro-1H- |
| | indazol-1-yl)benzamide; |
| 532 | 4-[6,6-dimethyl-4-oxo-3-(trifluoromethyl)-4,5,6,7-tetrahydro-1H-indazol-1-yl]-2-{[4-(2- |
| | oxoethoxy)cyclohexyl]amino}benzamide; |
| 533 | 2-{[4-(2,3-dihydroxypropoxy)cyclohexyl]amino}-4-[6,6-dimethyl-4-oxo-3- |
| | (trifluoromethyl)-4,5,6,7-tetrahydro-1H-indazol-1-yl]benzamide; |
| 534 | 4-[6,6-dimethyl-4-oxo-3-(trifluoromethyl)-4,5,6,7-tetrahydro-1H-indazol-1-yl]-2-({2- |
| | [(isopropylsulfonyl)amino]ethyl}amino)benzamide; |
| 535 | 4-[6,6-dimethyl-4-oxo-3-(trifluoromethyl)-4,5,6,7-tetrahydro-1H-indazol-1-yl]-2-{[4-(2- |
| | hydroxyethoxy)cyclohexyl]amino}benzamide; |
| 536 | {[4-({2-(aminocarbonyl)-5-[6,6-dimethyl-4-oxo-3-(trifluoromethyl)-4,5,6,7-tetrahydro- |
| | 1H-indazol-1-yl]phenyl}amino)cyclohexyl]oxy}acetic acid; |
| 537 | 4-[6,6-dimethyl-4-oxo-3-(trifluoromethyl)-4,5,6,7-tetrahydro-1H-indazol-1-yl]-2-({2- |
| | [(phenylsulfonyl)amino]ethyl}amino)benzamide; |
| 538 | 2-{[2-(morpholin-4-ylsulfonyl)ethyl]amino}-4-(3,6,6-trimethyl-4-oxo-4,5,6,7-tetrahydro- |
| | 1H-indazol-1-yl)benzamide; |
| 539 | 4-[3-(2-aminoethyl)-6,6-dimethyl-4-oxo-4,5,6,7-tetrahydro-1H-indazol-1-yl]benzamide; |
| 540 | 2-[6,6-dimethyl-4-oxo-3-(trifluoromethyl)-4,5,6,7-tetrahydro-1H-indazol-1-yl]-4- |
| | {methyl[tetrahydrofuran-3-yl]amino}benzamide; |
| 541 | 2-[6,6-dimethyl-4-oxo-3-(trifluoromethyl)-4,5,6,7-tetrahydro-1H-indazol-1-yl]-4-[(2- |
| | methoxy-1-methylethyl)amino]benzamide; |
| 542 | 4-bromo-2-[6,6-dimethyl-4-oxo-3-(trifluoromethyl)-4,5,6,7-tetrahydro-1H-indazol-1- |
| | yl]benzamide; |
| 543 | 4-(3-ethyl-6,6-dimethyl-4-oxo-4,5,6,7-tetrahydro-1H-indazol-1-yl)-2-[(4- |
| | hydroxycyclohexyl)amino]benzamide; |
| 544 | 4-[6,6-dimethyl-4-oxo-3-(trifluoromethyl)-4,5,6,7-tetrahydro-1H-indazol-1-yl]-2-[(4-{[2- |
| | (hydroxyimino)ethyl]oxy}cyclohexyl)amino]benzamide; |
| 545 | 2-[(4-hydroxycyclohexyl)amino]-4-(3-isobutyl-6,6-dimethyl-4-oxo-4,5,6,7-tetrahydro- |
| | 1H-indazol-1-yl)benzamide; |
| 546 | 4-(3-cyclopropyl-6,6-dimethyl-4-oxo-4,5,6,7-tetrahydro-1H-indazol-1-yl)-2-[(4- |
| | hydroxycyclohexyl)amino]benzamide; |
| 547 | 4-[3-(aminomethyl)-6,6-dimethyl-4-oxo-4,5,6,7-tetrahydro-1H-indazol-1-yl]-2-[(4- |
| | hydroxycyclohexyl)amino]benzamide methanesulfonate (salt); |
| 548 | 2-[(1-methyl-2-oxo-2-piperidin-1-ylethyl)amino]-4-(3,6,6-trimethyl-4-oxo-4,5,6,7- |
| | tetrahydro-1H-indazol-1-yl)benzamide; |
| 549 | 4-[3-(2-aminoethyl)-6,6-dimethyl-4-oxo-4,5,6,7-tetrahydro-1H-indazol-1-yl]-2-[(4- |
| | hydroxycyclohexyl)amino]benzamide; |
| 550 | 2-[(4-hydroxycyclohexyl)amino]-4-(3-isopropyl-6,6-dimethyl-4-oxo-4,5,6,7-tetrahydro- |
| | 1H-indazol-1-yl)benzamide; |
| 551 | 2-[6,6-dimethyl-4-oxo-3-(trifluoromethyl)-4,5,6,7-tetrahydro-1H-indazol-1-yl]-4- |
| | (tetrahydrofuran-3-ylamino)benzamide; |
| 552 | 4-[3-(cyclopropylmethyl)-6,6-dimethyl-4-oxo-4,5,6,7-tetrahydro-1H-indazol-1-yl]-2-[(4- |
| | hydroxycyclohexyl)amino]benzamide; |
| 553 | 2-[(4-hydroxycyclohexyl)amino]-4-(2,3,6,6-tetramethyl-4-oxo-4,5,6,7-tetrahydro-1H- |
| | indol-1-yl)benzamide; |
| 554 | 4-[3-(cyclopropylmethyl)-6,6-dimethyl-4-oxo-4,5,6,7-tetrahydro-1H-indazol-1- |
| | yl]benzamide; |
| 555 | 2-{[2-(dimethylamino)-2-oxoethyl]amino}-4-(3,6,6-trimethyl-4-oxo-4,5,6,7-tetrahydro- |
| | 1H-indazol-1-yl)benzamide; |
| 556 | 2-fluoro-6-(tetrahydro-2H-pyran-4-ylamino)-4-(2,3,6,6-tetramethyl-4-oxo-4,5,6,7- |
| | tetrahydro-1H-indol-1-yl)benzamide; |
| 557 | 4-(6,6-dimethyl-4-oxo-3-(trifluoromethyl)-4,5,6,7-tetrahydro-1H-indazol-1-yl)-2-fluoro-6- |
| | (4-hydroxycyclohexylamino)benzamide; |
| 558 | 2-fluoro-6-(4-methoxycyclohexylamino)-4-(3,6,6-trimethyl-4-oxo-4,5,6,7-tetrahydro-1H- |
| | indazol-1-yl)benzamide; |
| 559 | 4-(3-ethyl-6,6-dimethyl-4-oxo-4,5,6,7-tetrahydro-1H-indazol-1-yl)-3-fluoro-2-(2- |
| | hydroxycyclohexylamino)benzamide; |
| 560 | 4-(3-ethyl-6,6-dimethyl-4-oxo-4,5,6,7-tetrahydro-1H-indazol-1-yl)-2,3,5-trifluoro-6-(4- |
| | hydroxycyclohexylamino)benzamide; |
| 561 | 2-fluoro-6-(2-oxoazepan-3-ylamino)-4-(3,6,6-trimethyl-4-oxo-4,5,6,7-tetrahydro-1H- |
| | indazol-1-yl)benzamide; |
| 562 | 2-(1,3-dihydroxypropan-2-ylamino)-6-fluoro-4-(3,6,6-trimethyl-4-oxo-4,5,6,7- |
| | tetrahydro-1H-indazol-1-yl)benzamide; |
| 563 | 2-fluoro-6-(tetrahydrofuran-3-ylamino)-4-(3,6,6-trimethyl-4-oxo-4,5,6,7-tetrahydro-1H- |
| | indol-1-yl)benzamide; |
| 564 | 4-(3-(cyclopropylmethyl)-6,6-dimethyl-4-oxo-4,5,6,7-tetrahydro-1H-indazol-1-yl)-5- |
| | fluoro-2-(tetrahydrofuran-3-ylamino)benzamide; |
| 565 | 2-(2-aminocyclohexylamino)-6-fluoro-4-(3,6,6-trimethyl-4-oxo-4,5,6,7-tetrahydro-1H- |
| | indazol-1-yl)benzamide; |
| 566 | 2-fluoro-6-(2-(neopentylamino)cyclohexylamino)-4-(3,6,6-trimethyl-4-oxo-4,5,6,7- |
| | tetrahydro-1H-indazol-1-yl)benzamide; |
| 567 | 2-(2-((1H-imidazol-2-yl)methylthio)ethylamino)-4-(3-ethyl-6,6-dimethyl-4-oxo-4,5,6,7- |
| | tetrahydro-1H-indazol-1-yl)-6-fluorobenzamide; |
| 568 | 2-(cyclopent-3-enylamino)-6-fluoro-4-(3,6,6-trimethyl-4-oxo-4,5,6,7-tetrahydro-1H- |
| | indazol-1-yl)benzamide; |
| 569 | 2-fluoro-6-(2-hydroxycyclopentylamino)-4-(3,6,6-trimethyl-4-oxo-4,5,6,7-tetrahydro-1H- |
| | indol-1-yl)benzamide; |
| 570 | 2-(cyclobutylamino)-4-(3-ethyl-6,6-dimethyl-4-oxo-4,5,6,7-tetrahydro-1H-indazol-1-yl)- |
| | 6-fluorobenzamide; |
| 571 | 4-(3-(cyclopropylmethyl)-6,6-dimethyl-4-oxo-4,5,6,7-tetrahydro-1H-indazol-1-yl)-2- |
| | fluoro-6-(tetrahydro-2H-pyran-4-ylamino)benzamide; |
| 572 | 4-(3-ethyl-6,6-dimethyl-4-oxo-4,5,6,7-tetrahydro-1H-indazol-1-yl)-2-fluoro-6- |
| | (tetrahydro-2H-pyran-4-ylamino)benzamide; |
| 573 | 2-fluoro-6-(tetrahydro-2H-pyran-4-ylamino)-4-(3,6,6-trimethyl-4-oxo-4,5,6,7-tetrahydro- |
| | 1H-indazol-1-yl)benzamide; |
| 574 | 4-(3-ethyl-6,6-dimethyl-4-oxo-4,5,6,7-tetrahydro-1H-indazol-1-yl)-3-fluoro-2-(4- |
| | hydroxycyclohexylamino)benzamide; |
| 575 | 2-fluoro-6-(tetrahydrofuran-3-ylamino)-4-(3,6,6-trimethyl-4-oxo-4,5,6,7-tetrahydro-1H- |
| | indazol-1-yl)benzamide; |
| 576 | 2-fluoro-6-(tetrahydrofuran-3-ylamino)-4-(3,6,6-trimethyl-4-oxo-4,5,6,7-tetrahydro-1H- |
| | indazol-1-yl)benzamide; |
| 577 | 2-(2-aminocyclohexylamino)-4-(3-ethyl-6,6-dimethyl-4-oxo-4,5,6,7-tetrahydro-1H- |
| | indazol-1-yl)-6-fluorobenzamide; |
| 578 | 4-(3-(cyclopropylmethyl)-6,6-dimethyl-4-oxo-4,5,6,7-tetrahydro-1H-indazol-1-yl)-2- |
| | fluoro-6-(2-hydroxycyclopentylamino)benzamide; |
| 579 | 4-(3-(cyclopropylmethyl)-6,6-dimethyl-4-oxo-4,5,6,7-tetrahydro-1H-indazol-1-yl)-2- |
| | fluoro-6-(2-hydroxycyclohexylamino)benzamide; |
| 580 | 4-(3-(cyclopropylmethyl)-6,6-dimethyl-4-oxo-4,5,6,7-tetrahydro-1H-indazol-1-yl)-2- |
| | fluoro-6-(4-hydroxycyclohexylamino)benzamide; |
| 581 | 4-(3-(cyclopropylmethyl)-6,6-dimethyl-4-oxo-4,5,6,7-tetrahydro-1H-indazol-1-yl)-5- |
| | fluoro-2-(4-hydroxycyclohexylamino)benzamide; |
| 582 | 4-(3-ethyl-6,6-dimethyl-4-oxo-4,5,6,7-tetrahydro-1H-indazol-1-yl)-5-fluoro-2-(4- |
| | hydroxycyclohexylamino)benzamide; |
| 583 | 2-fluoro-6-(2-hydroxycyclopentylamino)-4-(3,6,6-trimethyl-4-oxo-4,5,6,7-tetrahydro-1H- |
| | indazol-1-yl)benzamide; |
| 584 | 2-(cyclopropylmethylamino)-4-(3-ethyl-6,6-dimethyl-4-oxo-4,5,6,7-tetrahydro-1H- |
| | indazol-1-yl)-6-fluorobenzamide; |
| 585 | 2-fluoro-6-(2-hydroxycyclohexylamino)-4-(3,6,6-trimethyl-4-oxo-4,5,6,7-tetrahydro-1H- |
| | indazol-1-yl)benzamide; |
| 586 | 4-(3-ethyl-6,6-dimethyl-4-oxo-4,5,6,7-tetrahydro-1H-indazol-1-yl)-2-fluoro-6- |
| | (tetrahydrofuran-3-ylamino)benzamide; |
| 587 | 4-(3-ethyl-6,6-dimethyl-4-oxo-4,5,6,7-tetrahydro-1H-indazol-1-yl)-2-fluoro-6-(2- |
| | hydroxycyclohexylamino)benzamide; |
| 588 | 4-(3-ethyl-6,6-dimethyl-4-oxo-4,5,6,7-tetrahydro-1H-indazol-1-yl)-2-fluoro-6- |
| | (tetrahydrofuran-3-ylamino)benzamide; |
| 589 | 4-(3-(cyclopropylmethyl)-6,6-dimethyl-4-oxo-4,5,6,7-tetrahydro-1H-indazol-1-yl)-2- |
| | fluoro-6-(tetrahydrofuran-3-ylamino)benzamide; |
| 590 | 4-(3-(cyclopropylmethyl)-6,6-dimethyl-4-oxo-4,5,6,7-tetrahydro-1H-indazol-1-yl)-2- |
| | fluoro-6-(tetrahydrofuran-3-ylamino)benzamide; |
| 591 | 4-(3-ethyl-6,6-dimethyl-4-oxo-4,5,6,7-tetrahydro-1H-indazol-1-yl)-3-fluoro-2- |
| | (tetrahydro-2H-pyran-4-ylamino)benzamide; |
| 592 | 4-(3-(cyclopropylmethyl)-6,6-dimethyl-4-oxo-4,5,6,7-tetrahydro-1H-indazol-1-yl)-3- |
| | fluoro-2-(tetrahydro-2H-pyran-4-ylamino)benzamide; |
| 593 | 4-(3-ethyl-6,6-dimethyl-4-oxo-4,5,6,7-tetrahydro-1H-indazol-1-yl)-3-fluoro-2-(2- |
| | hydroxyethylamino)benzamide; |
| 594 | 4-(3-(cyclopropylmethyl)-6,6-dimethyl-4-oxo-4,5,6,7-tetrahydro-1H-indazol-1-yl)-3- |
| | fluoro-2-(4-hydroxycyclohexylamino)benzamide; |
| 595 | 4-(3-(cyclopropylmethyl)-6,6-dimethyl-4-oxo-4,5,6,7-tetrahydro-1H-indazol-1-yl)-3- |
| | fluoro-2-(2-hydroxycyclohexylamino)benzamide; |
| 596 | 4-(3-ethyl-6,6-dimethyl-4-oxo-4,5,6,7-tetrahydro-1H-indazol-1-yl)-2-fluoro-6-(2- |
| | hydroxycyclopentylamino)benzamide; |
| 597 | 4-(3-(cyclopropylmethyl)-6,6-dimethyl-4-oxo-4,5,6,7-tetrahydro-1H-indazol-1-yl)-5- |
| | fluoro-2-(tetrahydro-2H-pyran-4-ylamino)benzamide; |
| 598 | 4-(3-ethyl-6,6-dimethyl-4-oxo-4,5,6,7-tetrahydro-1H-indazol-1-yl)-5-fluoro-2- |
| | (tetrahydro-2H-pyran-4-ylamino)benzamide; |
| 599 | 4-(3-ethyl-6,6-dimethyl-4-oxo-4,5,6,7-tetrahydro-1H-indazol-1-yl)-5-fluoro-2- |
| | (tetrahydrofuran-3-ylamino)benzamide; |
| 600 | 4-(3-(cyclopropylmethyl)-6,6-dimethyl-4-oxo-4,5,6,7-tetrahydro-1H-indazol-1-yl)-5- |
| | fluoro-2-(2-hydroxycyclohexylamino)benzamide; |
| 601 | 4-(3-(cyclopropylmethyl)-6,6-dimethyl-4-oxo-4,5,6,7-tetrahydro-1H-indazol-1-yl)-5- |
| | fluoro-2-(2-hydroxycyclopentylamino)benzamide; |
| 602 | 4-(3-ethyl-6,6-dimethyl-4-oxo-4,5,6,7-tetrahydro-1H-indazol-1-yl)-5-fluoro-2-(2- |
| | hydroxycyclopentylamino)benzamide; |
| 603 | 4-(3-ethyl-6,6-dimethyl-4-oxo-4,5,6,7-tetrahydro-1H-indazol-1-yl)-3,5-difluoro-2- |
| | (tetrahydro-2H-pyran-4-ylamino)benzamide; |
| 604 | 4-(3-ethyl-6,6-dimethyl-4-oxo-4,5,6,7-tetrahydro-1H-indazol-1-yl)-3,5-difluoro-2- |
| | (tetrahydrofuran-3-ylamino)benzamide; |
| 605 | 4-(3-(cyclopropylmethyl)-6,6-dimethyl-4-oxo-4,5,6,7-tetrahydro-1H-indazol-1-yl)-3,5- |
| | difluoro-2-(tetrahydrofuran-3-ylamino)benzamide; |
| 606 | 4-(3-(cyclopropylmethyl)-6,6-dimethyl-4-oxo-4,5,6,7-tetrahydro-1H-indazol-1-yl)-3,5- |
| | difluoro-2-(tetrahydro-2H-pyran-4-ylamino)benzamide; |
| 607 | 4-(3-ethyl-6,6-dimethyl-4-oxo-4,5,6,7-tetrahydro-1H-indazol-1-yl)-2-fluoro-6-(2- |
| | (neopentylamino)cyclohexylamino)benzamide; |
| 608 | 4-(3-ethyl-6,6-dimethyl-4-oxo-4,5,6,7-tetrahydro-1H-indazol-1-yl)-2-fluoro-6-(4- |
| | (neopentylamino)cyclohexylamino)benzamide; |
| 609 | 4-(3-ethyl-6,6-dimethyl-4-oxo-4,5,6,7-tetrahydro-1H-indazol-1-yl)-2-fluoro-6-(2- |
| | oxotetrahydrofuran-3-ylamino)benzamide; |
| 610 | 4-(3-(cyclopropylmethyl)-6,6-dimethyl-4-oxo-4,5,6,7-tetrahydro-1H-indazol-1-yl)-3,5- |
| | difluoro-2-(4-hydroxycyclohexylamino)benzamide; |
| 611 | methyl 2-(2-carbamoyl-5-(3-ethyl-6,6-dimethyl-4-oxo-4,5,6,7-tetrahydro-1H-indazol-1- |
| | yl)-3-fluorophenylamino)-4-hydroxybutanoate; |
| 612 | 2-(cyclopent-3-enylamino)-4-(3-ethyl-6,6-dimethyl-4-oxo-4,5,6,7-tetrahydro-1H- |
| | indazol-1-yl)-6-fluorobenzamide; |
| 613 | 2-(1-acetylpiperidin-4-ylamino)-4-(3-ethyl-6,6-dimethyl-4-oxo-4,5,6,7-tetrahydro-1H- |
| | indazol-1-yl)-6-fluorobenzamide; |
| 614 | 4-(3-(cyclopropylmethyl)-6,6-dimethyl-4-oxo-4,5,6,7-tetrahydro-1H-indazol-1-yl)-2- |
| | fluoro-6-(2-oxotetrahydrofuran-3-ylamino)benzamide; |
| 615 | 2-fluoro-6-(4-hydroxycyclohexylamino)-4-(3,6,6-trimethyl-4-oxo-4,5,6,7-tetrahydro-1H- |
| | indazol-1-yl)benzamide; |
| 616 | 2-fluoro-6-(2-oxotetrahydrofuran-3-ylamino)-4-(3,6,6-trimethyl-4-oxo-4,5,6,7- |
| | tetrahydro-1H-indazol-1-yl)benzamide; |
| 617 | 4-(3-ethyl-6,6-dimethyl-4-oxo-4,5,6,7-tetrahydro-1H-indazol-1-yl)-2-fluoro-6-(2- |
| | oxoazepan-3-ylamino)benzamide; |
| 618 | 2-fluoro-6-(tetrahydrofuran-3-ylamino)-4-(2,3,6,6-tetramethyl-4-oxo-4,5,6,7-tetrahydro- |
| | 1H-indol-1-yl)benzamide; |
| 619 | 2-fluoro-6-(tetrahydro-2H-pyran-4-ylamino)-4-(3,6,6-trimethyl-4-oxo-4,5,6,7-tetrahydro- |
| | 1H-indol-1-yl)benzamide; |
| 620 | 2-(2,2-dimethoxyethylamino)-4-(3-ethyl-6,6-dimethyl-4-oxo-4,5,6,7-tetrahydro-1H- |
| | indazol-1-yl)-6-fluorobenzamide; |
| 621 | 4-(3-ethyl-6,6-dimethyl-4-oxo-4,5,6,7-tetrahydro-1H-indazol-1-yl)-2-fluoro-6-(4- |
| | hydroxycyclohexylamino)benzamide; |
| 622 | 2-fluoro-6-(4-hydroxycyclohexylamino)-4-(3,6,6-trimethyl-4-oxo-4,5,6,7-tetrahydro-1H- |
| | indol-1-yl)benzamide; |
| 623 | 4-(3-ethyl-6,6-dimethyl-4-oxo-4,5,6,7-tetrahydro-1H-indazol-1-yl)-2-fluoro-6-(4- |
| | methoxycyclohexylamino)benzamide; |
| 624 | 2-(4-(cyclopropylmethylamino)cyclohexylamino)-4-(6,6-dimethyl-4-oxo-3- |
| | (trifluoromethyl)-4,5,6,7-tetrahydro-1H-indazol-1-yl)benzamide; |
| 625 | 2-(1-(2-(isobutylamino)ethyl)-1H-pyrazol-4-ylamino)-4-(3,6,6-trimethyl-4-oxo-4,5,6,7- |
| | tetrahydro-1H-indazol-1-yl)benzamide; |
| 626 | 4-(6,6-dimethyl-4-oxo-3-(trifluoromethyl)-4,5,6,7-tetrahydro-1H-indazol-1-yl)-2- |
| | (piperidin-3-ylamino)benzamide; |
| 627 | 4-(6,6-dimethyl-4-oxo-3-(trifluoromethyl)-4,5,6,7-tetrahydro-1H-indazol-1-yl)-2-(1- |
| | neopentylpiperidin-3-ylamino)benzamide; |
| 628 | 4-(6,6-dimethyl-4-oxo-3-(trifluoromethyl)-4,5,6,7-tetrahydro-1H-indazol-1-yl)-2-(1- |
| | (prop-2-ynyl)pyrrolidin-3-ylamino)benzamide; |
| 629 | 2-(4-aminocyclohexylamino)-4-(6,6-dimethyl-4-oxo-3-(trifluoromethyl)-4,5,6,7- |
| | tetrahydro-1H-indazol-1-yl)benzamide; |
| 630 | (4-(2-carbamoyl-5-(6,6-dimethyl-4-oxo-3-(trifluoromethyl)-4,5,6,7-tetrahydro-1H- |
| | indazol-1-yl)phenylamino)cyclohexyl) 2-amino-3-hydroxypropanoate; |
| 631 | 4-(2-carbamoyl-5-(6,6-dimethyl-4-oxo-3-(trifluoromethyl)-4,5,6,7-tetrahydro-1H- |
| | indazol-1-yl)phenylamino)cyclohexyl 2-(dimethylamino)acetate; |
| 632 | 4-(6,6-dimethyl-4-oxo-3-(trifluoromethyl)-4,5,6,7-tetrahydro-1H-indazol-1-yl)-2- |
| | (pyrimidin-2-ylamino)benzamide; |
| 633 | 4-(6,6-dimethyl-4-oxo-3-(trifluoromethyl)-4,5,6,7-tetrahydro-1H-indazol-1-yl)-2- |
| | (pyrimidin-4-ylamino)benzamide; |
| 634 | 4-(6,6-dimethyl-4-oxo-3-(trifluoromethyl)-4,5,6,7-tetrahydro-1H-indazol-1-yl)-2- |
| | (pyrimidin-5-ylamino)benzamide; |
| 635 | 4-(3-(difluoromethyl)-6,6-dimethyl-4-oxo-4,5,6,7-tetrahydro-1H-indazol-1-yl)-2-(1- |
| | (prop-2-ynyl)piperidin-4-ylamino)benzamide; |
| 636 | 2-(1-cyclopropylpiperidin-4-ylamino)-4-(3-(difluoromethyl)-;6,6-dimethyl-4-oxo-4;,5,6,7- |
| | tetrahydro-1H-indazol-1-yl)benzamide; |
| 637 | 4-(3-(difluoromethyl)-6,6-dimethyl-4-oxo-4,5,6,7-tetrahydro-1H-indazol-1-yl)-2-(1-(2- |
| | methoxyethyl)piperidin-4-ylamino)benzamide; |
| 638 | 4-(6,6-dimethyl-4-oxo-3-(trifluoromethyl)-4,5,6,7-tetrahydro-1H-indazol-1-yl)-2-(2-(2- |
| | hydroxyethoxy)pyridin-4-ylamino)benzamide; |
| 639 | 2-(1-(2-(methylamino)ethyl)-1H-pyrazol-4-ylamino)-4-(3,6,6-trimethyl-4-oxo-4,5,6,7- |
| | tetrahydro-1H-indazol-1-yl)benzamide; |
| 640 | 4-(6,6-dimethyl-4-oxo-3-(trifluoromethyl)-4,5,6,7-tetrahydro-1H-indazol-1-yl)-2-(4- |
| | (prop-2-ynylamino)cyclohexylamino)benzamide; |
| 641 | 4-(6,6-dimethyl-4-oxo-3-(trifluoromethyl)-4,5,6,7-tetrahydro-1H-indazol-1-yl)-2-(1- |
| | (prop-2-ynyl)piperidin-4-ylamino)benzamide; |
| 642 | 2-(azetidin-3-ylamino)-4-(6,6-dimethyl-4-oxo-3-(trifluoromethyl)-4,5,6,7-tetrahydro-1H- |
| | indazol-1-yl)benzamide; |
| 643 | 2-(2-aminocyclohexylamino)-4-(6,6-dimethyl-4-oxo-3-(trifluoromethyl)-4,5,6,7- |
| | tetrahydro-1H-indazol-1-yl)benzamide; |
| 644 | 4-(6,6-dimethyl-4-oxo-3-(trifluoromethyl)-4,5,6,7-tetrahydro-1H-indazol-1-yl)-2-(2- |
| | (diprop-2-ynylamino)ethylamino)benzamide; |
| 645 | 4-(6,6-dimethyl-4-oxo-3-(trifluoromethyl)-4,5,6,7-tetrahydro-1H-indazol-1-yl)-2-(2- |
| | (prop-2-ynylamino)ethylamino)benzamide; |
| 646 | 4-(6,6-dimethyl-4-oxo-3-(trifluoromethyl)-4,5,6,7-tetrahydro-1H-indazol-1-yl)-2-(2- |
| | (prop-2-ynylamino)cyclohexylamino)benzamide; |
| 647 | 2-(1-allylpiperidin-4-ylamino)-4-(6,6-dimethyl-4-oxo-3-(trifluoromethyl)-4,5,6,7- |
| | tetrahydro-1H-indazol-1-yl)benzamide; |
| 648 | 2-(2-aminocyclohexylamino)-4-(6,6-dimethyl-4-oxo-3-(trifluoromethyl)-4,5,6,7- |
| | tetrahydro-1H-indazol-1-yl)benzamide; |
| 649 | 2-(2-aminocyclohexylamino)-4-(6,6-dimethyl-4-oxo-3-(trifluoromethyl)-4,5,6,7- |
| | tetrahydro-1H-indazol-1-yl)benzamide; |
| 650 | 4-(6,6-dimethyl-4-oxo-3-(trifluoromethyl)-4,5,6,7-tetrahydro-1H-indazol-1-yl)-2-(1- |
| | (prop-2-ynyl)piperidin-3-ylamino)benzamide; |
| 651 | 2-(1-allylpiperidin-3-ylamino)-4-(6,6-dimethyl-4-oxo-3-(trifluoromethyl)-4,5,6,7- |
| | tetrahydro-1H-indazol-1-yl)benzamide; |
| 652 | 4-(6,6-dimethyl-4-oxo-3-(trifluoromethyl)-4,5,6,7-tetrahydro-1H-indazol-1-yl)-2- |
| | (piperidin-3-ylamino)benzamide; |
| 653 | 4-(6,6-dimethyl-4-oxo-3-(trifluoromethyl)-4,5,6,7-tetrahydro-1H-indazol-1-yl)-2-(2- |
| | (pyrimidin-2-ylthio)ethylamino)benzamide; |
| 654 | 4-(6,6-dimethyl-4-oxo-3-(trifluoromethyl)-4,5,6,7-tetrahydro-1H-indazol-1-yl)-2-(1- |
| | (prop-2-ynyl)piperidin-3-ylamino)benzamide; |
| 655 | 2-(1-(cyclopropylmethyl)piperidin-3-ylamino)-4-(6,6-dimethyl-4-oxo-3-(trifluoromethyl)- |
| | 4,5,6,7-tetrahydro-1H-indazol-1-yl)benzamide; |
| 656 | 2-(1-(cyclopropylmethyl)piperidin-4-ylamino)-4-(6,6-dimethyl-4-oxo-3-(trifluoromethyl)- |
| | 4,5,6,7-tetrahydro-1H-indazol-1-yl)benzamide; |
| 657 | 2-(3-aminocyclohexylamino)-4-(6,6-dimethyl-4-oxo-3-(trifluoromethyl)-4,5,6,7- |
| | tetrahydro-1H-indazol-1-yl)benzamide; |
| 658 | 2-(1-(2-(tert-butylamino)ethyl)-1H-pyrazol-4-ylamino)-4-(3,6,6-trimethyl-4-oxo-4,5,6,7- |
| | tetrahydro-1H-indazol-1-yl)benzamide; |
| 659 | 4-(6,6-dimethyl-4-oxo-3-(trifluoromethyl)-4,5,6,7-tetrahydro-1H-indazol-1-yl)-2- |
| | (pyridazin-4-ylamino)benzamide; |
| 660 | 2-(1-(cyclopropylmethyl)piperidin-3-ylamino)-4-(6,6-dimethyl-4-oxo-3-(trifluoromethyl)- |
| | 4,5,6,7-tetrahydro-1H-indazol-1-yl)benzamide; |
| 661 | 2-(2-(cyclopropylmethylamino)cyclohexylamino)-4-(6,6-dimethyl-4-oxo-3- |
| | (trifluoromethyl)-4,5,6,7-tetrahydro-1H-indazol-1-yl)benzamide; |
| 662 | 2-(2-(1H-imidazol-2-ylthio)ethylamino)-4-(6,6-dimethyl-4-oxo-3-(trifluoromethyl)- |
| | 4,5,6,7-tetrahydro-1H-indazol-1-yl)benzamide; |
| 663 | 4-(6,6-dimethyl-4-oxo-3-(trifluoromethyl)-4,5,6,7-tetrahydro-1H-indazol-1-yl)-2-(2- |
| | (prop-2-ynylamino)cyclohexylamino)benzamide; |
| 664 | 4-(6,6-dimethyl-4-oxo-3-(trifluoromethyl)-4,5,6,7-tetrahydro-1H-indazol-1-yl)-2-(2- |
| | (diprop-2-ynylamino)cyclohexylamino)benzamide; |
| 665 | 2-(2-(cyclopropylmethylamino)cyclohexylamino)-4-(6,6-dimethyl-4-oxo-3- |
| | (trifluoromethyl)-4,5,6,7-tetrahydro-1H-indazol-1-yl)benzamide; |
| 666 | 2-(2-(bis(cyclopropylmethyl)amino)cyclohexylamino)-4-(6,6-dimethyl-4-oxo-3- |
| | (trifluoromethyl)-4,5,6,7-tetrahydro-1H-indazol-1-yl)benzamide; |
| 667 | 2-(2-(diethylamino)cyclohexylamino)-4-(6,6-dimethyl-4-oxo-3-(trifluoromethyl)-4,5,6,7- |
| | tetrahydro-1H-indazol-1-yl)benzamide; |
| 668 | 2-(2-(cyclopropylamino)cyclohexylamino)-4-(6,6-dimethyl-4-oxo-3-(trifluoromethyl)- |
| | 4,5,6,7-tetrahydro-1H-indazol-1-yl)benzamide; |
| 669 | 4-(6,6-dimethyl-4-oxo-3-(trifluoromethyl)-4,5,6,7-tetrahydro-1H-indazol-1-yl)-2-(4- |
| | (neopentylamino)cyclohexylamino)benzamide; |
| 670 | 4-(6,6-dimethyl-4-oxo-3-(trifluoromethyl)-4,5,6,7-tetrahydro-1H-indazol-1-yl)-2-(2- |
| | (neopentylamino)cyclohexylamino)benzamide; |
| 671 | 2-(4-hydroxycyclohexylamino)-4-(3-(hydroxymethyl)-6,6-dimethyl-4-oxo-4,5,6,7- |
| | tetrahydro-1H-indazol-1-yl)benzamide; |
| 672 | 2-(1-(2-hydroxyethyl)-1H-pyrazol-4-ylamino)-4-(3,6,6-trimethyl-4-oxo-4,5,6,7- |
| | tetrahydro-1H-indazol-1-yl)benzamide; |
| 673 | 4-(6,6-dimethyl-4-oxo-3-(pyridin-2-ylmethyl)-4,5,6,7-tetrahydro-1H-indazol-1-yl)-2- |
| | (tetrahydro-2H-pyran-4-ylamino)benzamide; |
| 674 | 4-(3-ethyl-6,6-dimethyl-4-oxo-4,5,6,7-tetrahydro-1H-indazol-1-yl)-2-(tetrahydrofuran-3- |
| | ylamino)benzamide; |
| 675 | 4-(6,6-dimethyl-4-oxo-3-(trifluoromethyl)-4,5,6,7-tetrahydro-1H-indazol-1-yl)-2-(2- |
| | hydroxypropylamino)benzamide; |
| 676 | 4-(6,6-dimethyl-4-oxo-3-(trifluoromethyl)-4,5,6,7-tetrahydro-1H-indazol-1-yl)-2-(4-(2- |
| | (methoxyimino)ethoxy)cyclohexylamino)benzamide; |
| 677 | 4-(6,6-dimethyl-4-oxo-3-(trifluoromethyl)-4,5,6,7-tetrahydro-1H-indazol-1-yl)-2-(4-(2- |
| | (phenoxyimino)ethoxy)cyclohexylamino)benzamide; |
| 678 | 2-(4-(2-hydroxyethoxy)cyclohexylamino)-4-(3,6,6-trimethyl-4-oxo-4,5,6,7-tetrahydro- |
| | 1H-indazol-1-yl)benzamide; |
| 679 | 2-(tetrahydrofuran-3-ylamino)-4-(3,6,6-trimethyl-4-oxo-4,5,6,7-tetrahydro-1H-indazol- |
| | 1-yl)benzamide; |
| 680 | 4-(6,6-dimethyl-4-oxo-3-(trifluoromethyl)-4,5,6,7-tetrahydro-1H-indazol-1-yl)-2-(4-(2- |
| | (hydroxyamino)-2-oxoethoxy)cyclohexylamino)benzamide; |
| 681 | 2-(4-(2-(2-hydroxyethoxy)ethoxy)cyclohexylamino)-4-(3,6,6-trimethyl-4-oxo-4,5,6,7- |
| | tetrahydro-1H-indazol-1-yl)benzamide; |
| 682 | 2-(cyclobutylamino)-4-(6,6-dimethyl-4-oxo-3-(trifluoromethyl)-4,5,6,7-tetrahydro-1H- |
| | indazol-1-yl)benzamide; |
| 683 | 2-(4-(2-hydroxyethoxy)cyclohexylamino)-4-(3,6,6-trimethyl-4-oxo-4,5,6,7-tetrahydro- |
| | 1H-indazol-1-yl)benzamide; |
| 684 | 4-(6,6-dimethyl-4-oxo-3-(trifluoromethyl)-4,5,6,7-tetrahydro-1H-indazol-1-yl)-2-(2- |
| | hydroxyethylamino)benzamide; |
| 685 | 2-(cyclobutylamino)-4-(3-ethyl-6,6-dimethyl-4-oxo-4,5,6,7-tetrahydro-1H-indazol-1- |
| | yl)benzamide;; |
| 686 | 2-(-4-hydroxycyclohexylamino)-4-(3-(3-methoxyphenyl)-6,6-dimethyl-4-oxo-4,5,6,7- |
| | tetrahydro-1H-indazol-1-yl)benzamide; |
| 687 | 4-(3-benzyl-6,6-dimethyl-4-oxo-4,5,6,7-tetrahydro-1H-indazol-1-yl)-2-(4- |
| | hydroxycyclohexylamino)benzamide; |
| 688 | 4-(6,6-dimethyl-4-oxo-3-(1H-pyrazol-4-yl)-4,5,6,7-tetrahydro-1H-indazol-1-yl)-2-(4- |
| | hydroxycyclohexylamino)benzamide; |
| 689 | 4-(6,6-dimethyl-4-oxo-3-(pyridin-3-ylmethyl)-4,5,6,7-tetrahydro-1H-indazol-1-yl)-2-(4- |
| | hydroxycyclohexylamino)benzamide; |
| 690 | 4-(6,6-dimethyl-4-oxo-3-(pyridin-2-ylmethyl)-4,5,6,7-tetrahydro-1H-indazol-1-yl)-2-(4- |
| | hydroxycyclohexylamino)benzamide; |
| 691 | 4-(3-(cyclopropylmethyl)-6,6-dimethyl-4-oxo-4,5,6,7-tetrahydro-1H-indazol-1-yl)-2- |
| | (tetrahydro-2H-pyran-4-ylamino)benzamide; |
| 692 | 4-(6,6-dimethyl-4-oxo-3-(trifluoromethyl)-4,5,6,7-tetrahydro-1H-indazol-1-yl)-2-(2- |
| | hydroxycyclohexylamino)benzamide; |
| 693 | 4-(6,6-dimethyl-4-oxo-3-(pyridin-2-ylmethyl)-4,5,6,7-tetrahydro-1H-indazol-1-yl)-2-(2- |
| | hydroxethylamino)benzamide; |
| 694 | 4-(3-benzyl-6,6-dimethyl-4-oxo-4,5,6,7-tetrahydro-1H-indazol-1-yl)-2-(2- |
| | hydroxyethylamino)benzamide; |
| 695 | 4-(3-(cyclopropylmethyl)-6,6-dimethyl-4-oxo-4,5,6,7-tetrahydro-1H-indazol-1-yl)-2-(2- |
| | hydroxyethylamino)benzamide; |
| 696 | 4-(3-benzyl-6,6-dimethyl-4-oxo-4,5,6,7-tetrahydro-1H-indazol-1-yl)-2-(tetrahydro-2H- |
| | pyran-4-ylamino)benzamide; |
| 697 | 4-(6,6-dimethyl-4-oxo-3-(trifluoromethyl)-4,5,6,7-tetrahydro-1H-indazol-1-yl)-2-(1- |
| | hydroxypropan-2-ylamino)benzamide; |
| 698 | 4-(6,6-dimethyl-4-oxo-3-(trifluoromethyl)-4,5,6,7-tetrahydro-1H-indazol-1-yl)-2-(1- |
| | hydroxypropan-2-ylamino)benzamide; |
| 699 | 4-(6,6-dimethyl-4-oxo-3-(trifluoromethyl)-4,5,6,7-tetrahydro-1H-indazol-1-yl)-2-(2- |
| | hydroxypropylamino)benzamide; |
| 700 | 4-(6,6-dimethyl-4-oxo-3-(pyridin-3-ylmethyl)-4,5,6,7-tetrahydro-1H-indazol-1-yl)-2- |
| | (tetrahydro-2H-pyran-4-ylamino)benzamide; |
| 701 | 4-(6,6-dimethyl-4-oxo-3-(pyridin-4-ylmethyl)-4,5,6,7-tetrahydro-1H-indazol-1-yl)-2-(2- |
| | hydroxyethylamino)benzamide; |
| 702 | 4-(6,6-dimethyl-4-oxo-3-(pyridin-3-ylmethyl)-4,5,6,7-tetrahydro-1H-indazol-1-yl)-2-(2- |
| | hydroxyethylamino)benzamide; |
| 703 | 4-(6,6-dimethyl-4-oxo-3-(thiophen-3-ylmethyl)-4,5,6,7-tetrahydro-1H-indazol-1-yl)-2-(2- |
| | hydroxyethylamino)benzamide; |
| 704 | 4-(6,6-dimethyl-4-oxo-3-(pyridin-4-ylmethyl)-4,5,6,7-tetrahydro-1H-indazol-1-yl)-2- |
| | (tetrahydro-2H-pyran-4-ylamino)benzamide; |
| 705 | 4-(6,6-dimethyl-4-oxo-3-(pyridin-4-ylmethyl)-4,5,6,7-tetrahydro-1H-indazol-1-yl)-2-(4- |
| | hydroxycyclohexylamino)benzamide; |
| 706 | 4-(6,6-dimethyl-4-oxo-3-(trifluoromethyl)-4,5,6,7-tetrahydro-1H-indazol-1-yl)-2-(2- |
| | hydroxycyclohexylamino)benzamide; |
| 707 | 4-(3-(cyclopropylmethyl)-6,6-dimethyl-4-oxo-4,5,6,7-tetrahydro-1H-indazol-1-yl)-2- |
| | (tetrahydrofuran-3-ylamino)benzamide; |
| 708 | 4-(6,6-dimethyl-4-oxo-3-(trifluoromethyl)-4,5,6,7-tetrahydro-1H-indazol-1-yl)-2-(2- |
| | hydroxycyclohexylamino)benzamide; |
| 709 | 4-(6,6-dimethyl-4-oxo-3-(trifluoromethyl)-4,5,6,7-tetrahydro-1H-indazol-1-yl)-2-(2- |
| | hydroxycyclopentylamino)benzamide; |
| 710 | 4-(6,6-dimethyl-4-oxo-3-(trifluoromethyl)-4,5,6,7-tetrahydro-1H-indazol-1-yl)-2-(2- |
| | hydroxycyclopentylamino)benzamide; |
| 711 | 4-(3-ethyl-6,6-dimethyl-4-oxo-4,5,6,7-tetrahydro-1H-indazol-1-yl)-2-(2- |
| | hydroxycyclohexylamino)benzamide; |
| 712 | 4-(3-(cyclopropylmethyl)-6,6-dimethyl-4-oxo-4,5,6,7-tetrahydro-1H-indazol-1-yl)-2-(2- |
| | hydroxycyclopentylamino)benzamide; |
| 713 | 4-(3-(cyclopropylmethyl)-6,6-dimethyl-4-oxo-4,5,6,7-tetrahydro-1H-indazol-1-yl)-2-(2- |
| | hydroxycyclohexylamino)benzamide; |
| 714 | 4-(3-ethyl-6,6-dimethyl-4-oxo-4,5,6,7-tetrahydro-1H-indazol-1-yl)-2-(2- |
| | hydroxycyclopentylamino)benzamide; |
| 715 | 2-(tetrahydrofuran-3-ylamino)-4-(3,6,6-trimethyl-4-oxo-4,5,6,7-tetrahydro-1H-indazol- |
| | 1-yl)benzamide; |
| 716 | 2-(2-hydroxycyclopentylamino)-4-(3,6,6-trimethyl-4-oxo-4,5,6,7-tetrahydro-1H-indazol- |
| | 1-yl)benzamide; |
| 717 | 2-(2-hydroxycyclohexylamino)-4-(3,6,6-trimethyl-4-oxo-4,5,6,7-tetrahydro-1H-indazol- |
| | 1-yl)benzamide; |
| 718 | 2-methyl-4-(3,6,6-trimethyl-4-oxo-4,5,6,7-tetrahydro-1H-indazol-1-yl)-1H- |
| | benzo[d]imidazole-7-carboxamide; |
| 719 | 4-(3,6,6-trimethyl-4-oxo-4,5,6,7-tetrahydro-1H-indazol-1-yl)-1H-benzo[d]imidazole-7- |
| | carboxamide; |
| 720 | 4-(3-ethyl-6,6-dimethyl-4-oxo-4,5,6,7-tetrahydro-1H-indazol-1-yl)-1H- |
| | benzo[d]imidazole-7-carboxamide; |
| 721 | 4-(3-ethyl-6,6-dimethyl-4-oxo-4,5,6,7-tetrahydro-1H-indazol-1-yl)-2-methyl-1H- |
| | benzo[d]imidazole-7-carboxamide; |
| 722 | 2-phenyl-4-(3,6,6-trimethyl-4-oxo-4,5,6,7-tetrahydro-1H-indazol-1-yl)-1H- |
| | benzo[d]imidazole-7-carboxamide; |
| 723 | 4-(3-ethyl-6,6-dimethyl-4-oxo-4,5,6,7-tetrahydro-1H-indazol-1-yl)-2-phenyl-1H- |
| | benzo[d]imidazole-7-carboxamide; |
| 724 | 5-(6,6-dimethyl-4-oxo-3-(trifluoromethyl)-4,5,6,7-tetrahydro-1H-indazol-1-yl)-3-(-4- |
| | hydroxycyclohexylamino)picolinamide; |
| 725 | 2-fluoro-6-(2-hydroxycyclohexylamino)-4-(3,6,6-trimethyl-4-oxo-4,5,6,7-tetrahydro-1H- |
| | indol-1-yl)benzamide; |
| 726 | 4-(3-ethyl-6,6-dimethyl-4-oxo-4,5,6,7-tetrahydro-1H-indazol-1-yl)-2-fluoro-6-(2- |
| | hydroxycyclohexylamino)benzamide; |
| 727 | 4-(3-ethyl-6,6-dimethyl-4-oxo-4,5,6,7-tetrahydro-1H-indazol-1-yl)-2-fluoro-6-(2- |
| | hydroxycyclopentylamino)benzamide; |
| 728 | 2-(cyclopentylamino)-4-(3-ethyl-6,6-dimethyl-4-oxo-4,5,6,7-tetrahydro-1H-indazol-1- |
| | yl)-6-fluorobenzamide; |
| 729 | 2-(2-carbamoyl-3-fluoro-5-(3,6,6-trimethyl-4-oxo-4,5,6,7-tetrahydro-1H-indol-1- |
| | yl)phenylamino)cyclohexyl 2-aminoacetate; |
| 730 | 2-(2-carbamoyl-3-fluoro-5-(3,6,6-trimethyl-4-oxo-4,5,6,7-tetrahydro-1H-indol-1- |
| | yl)phenylamino)cyclopentyl 2-aminoacetate; |
| 731 | 2-(2-carbamoyl-5-(3-ethyl-6,6-dimethyl-4-oxo-4,5,6,7-tetrahydro-1H-indazol-1-yl)-3- |
| | fluorophenylamino)cyclohexyl 2-aminoacetate; |
| 732 | 2-(2-carbamoyl-5-(3-ethyl-6,6-dimethyl-4-oxo-4,5,6,7-tetrahydro-1H-indazol-1-yl)-3- |
| | fluorophenylamino)cyclopentyl 2-aminoacetate; |
| 733 | 2-(2-carbamoyl-3-fluoro-5-(3,6,6-trimethyl-4-oxo-4,5,6,7-tetrahydro-1H-indazol-1- |
| | yl)phenylamino)cyclopentyl 2-aminoacetate; |
| 734 | 2-(2-carbamoyl-3-fluoro-5-(3,6,6-trimethyl-4-oxo-4,5,6,7-tetrahydro-1H-indazol-1- |
| | yl)phenylamino)cyclohexyl 2-aminoacetate; |
| 735 | 4-(2-carbamoyl-3-fluoro-5-(3,6,6-trimethyl-4-oxo-4,5,6,7-tetrahydro-1H-indol-1- |
| | yl)phenylamino)cyclohexyl 2-aminoacetate; |
| 736 | 4-(2-carbamoyl-5-(3-ethyl-6,6-dimethyl-4-oxo-4,5,6,7-tetrahydro-1H-indazol-1-yl)-3- |
| | fluorophenylamino)cyclohexyl 2-aminoacetate; |
| 737 | 4-(2-carbamoyl-3-fluoro-5-(3,6,6-trimethyl-4-oxo-4,5,6,7-tetrahydro-1H-indazol-1- |
| | yl)phenylamino)cyclohexyl 2-aminoacetate; |
| 738 | 2-fluoro-6-(6-oxotetrahydro-2H-pyran-3-ylamino)-4-(3,6,6-trimethyl-4-oxo-4,5,6,7- |
| | tetrahydro-1H-indazol-1-yl)benzamide; |
| 739 | 2-fluoro-6-(6-oxotetrahydro-2H-pyran-3-ylamino)-4-(3,6,6-trimethyl-4-oxo-4,5,6,7- |
| | tetrahydro-1H-indazol-1-yl)benzamide; |
| 740 | 2-fluoro-6-(4-hydroxycyclohexylamino)-4-(3,6,6-trimethyl-4-oxo-4,5,6,7-tetrahydro-1H- |
| | indazol-1-yl)benzamide; |
| 741 | 4-(3-ethyl-6,6-dimethyl-4-oxo-4,5,6,7-tetrahydro-1H-indol-1-yl)-2-fluoro-6-(4- |
| | hydroxycyclohexylamino)benzamide; |
| 742 | 2-fluoro-6-(oxetan-3-ylamino)-4-(3,6,6-trimethyl-4-oxo-4,5,6,7-tetrahydro-1H-indazol-1- |
| | yl)benzamide; |
| 743 | 2-(cyclopentylamino)-6-fluoro-4-(3,6,6-trimethyl-4-oxo-4,5,6,7-tetrahydro-1H-indazol-1- |
| | yl)benzamide; |
| 744 | 2-(cycloheptylamino)-6-fluoro-4-(3,6,6-trimethyl-4-oxo-4,5,6,7-tetrahydro-1H-indazol-1- |
| | yl)benzamide; |
| 745 | 4-(2-carbamoyl-5-(6,6-dimethyl-4-oxo-3-(trifluoromethyl)-4,5,6,7-tetrahydro-1H- |
| | indazol-1-yl)-3-fluorophenylamino)cyclohexyl 2-aminoacetate; |
| 746 | 4-(6,6-dimethyl-4-oxo-3-(trifluoromethyl)-4,5,6,7-tetrahydro-1H-indazol-1-yl)-2-fluoro-6- |
| | (2-hydroxycyclopentylamino)benzamide; |
| 747 | 2-(2-carbamoyl-5-(6,6-dimethyl-4-oxo-3-(trifluoromethyl)-4,5,6,7-tetrahydro-1H- |
| | indazol-1-yl)-3-fluorophenylamino)cyclopentyl 2-aminoacetate; |
| 748 | 4-(6,6-dimethyl-4-oxo-3-(trifluoromethyl)-4,5,6,7-tetrahydro-1H-indazol-1-yl)-2-fluoro-6- |
| | (tetrahydro-2H-pyran-4-ylamino)benzamide; |
| 749 | 4-(6,6-dimethyl-4-oxo-3-(trifluoromethyl)-4,5,6,7-tetrahydro-1H-indazol-1-yl)-2-fluoro-6- |
| | (2-hydroxycyclohexylamino)benzamide; |
| 750 | 2-(2-carbamoyl-5-(6,6-dimethyl-4-oxo-3-(trifluoromethyl)-4,5,6,7-tetrahydro-1H- |
| | indazol-1-yl)-3-fluorophenylamino)cyclohexyl 2-aminoacetate; |
| 751 | 4-(6,6-dimethyl-4-oxo-3-(trifluoromethyl)-4,5,6,7-tetrahydro-1H-indazol-1-yl)-2-fluoro-6- |
| | (tetrahydrofuran-3-ylamino)benzamide; |
| 752 | 4-(3-ethyl-6,6-dimethyl-4-oxo-4,5,6,7-tetrahydro-1H-indazol-1-yl)-2-fluoro-6-(2- |
| | hydroxycyclopentylamino)benzamide; |
| 753 | 4-(3-ethyl-6,6-dimethyl-4-oxo-4,5,6,7-tetrahydro-1H-indol-1-yl)-2-fluoro-6- |
| | (tetrahydrofuran-3-ylamino)benzamide; |
| 754 | 2-(4,4-Difluoro-cyclohexylamino)-6-fluoro-4-(3,6,6-trimethyl-4-oxo-4,5,6,7-tetrahydro- |
| | indazol-1-yl)-benzamide; |
| 755 | 2-fluoro-6-(2-hydroxycyclopentylamino)-4-(3,6,6-trimethyl-4-oxo-4,5,6,7-tetrahydro-1H- |
| | indazol-1-yl)benzamide; |
| 756 | 2-(4,4-difluorocyclohexylamino)-6-fluoro-4-(3,6,6-trimethyl-4-oxo-4,5,6,7-tetrahydro- |
| | 1H-indazol-1-yl)benzamide; |
| 757 | 4-(6,6-dimethyl-4-oxo-3-(trifluoromethyl)-4,5,6,7-tetrahydro-1H-indol-1-yl)-2-fluoro-6- |
| | (2-hydroxycyclopentylamino)benzamide; |
| 758 | 2-(cyclopentylamino)-4-(6,6-dimethyl-4-oxo-3-(trifluoromethyl)-4,5,6,7-tetrahydro-1H- |
| | indazol-1-yl)-6-fluorobenzamide; |
| 759 | 2-(cyclohexylamino)-6-fluoro-4-(3,6,6-trimethyl-4-oxo-4,5,6,7-tetrahydro-1H-indazol-1- |
| | yl)benzamide; |
| 760 | 2-(2-carbamoyl-5-(6,6-dimethyl-4-oxo-3-(trifluoromethyl)-4,5,6,7-tetrahydro-1H-indol-1- |
| | yl)-3-fluorophenylamino)cyclopentyl 2-aminoacetate methanesulfonate; |
| 761 | 4-(6,6-dimethyl-4-oxo-3-(trifluoromethyl)-4,5,6,7-tetrahydro-1H-indol-1-yl)-2-fluoro-6- |
| | (4-hydroxycyclohexylamino)benzamide; |
| 762 | 4-(6,6-dimethyl-4-oxo-3-(trifluoromethyl)-4,5,6,7-tetrahydro-1H-indol-1-yl)-2-fluoro-6- |
| | (2-hydroxycyclohexylamino)benzamide; |
| 763 | 2-(2-carbamoyl-5-(6,6-dimethyl-4-oxo-3-(trifluoromethyl)-4,5,6,7-tetrahydro-1H-indol-1- |
| | yl)-3-fluorophenylamino)cyclohexyl 2-aminoacetate methanesulfonate; |
| 764 | 4-(6,6-dimethyl-4-oxo-3-(trifluoromethyl)-4,5,6,7-tetrahydro-1H-indol-1-yl)-2-fluoro-6- |
| | (tetrahydro-2H-pyran-4-ylamino)benzamide; |
| 765 | 4-(6,6-dimethyl-4-oxo-3-(trifluoromethyl)-4,5,6,7-tetrahydro-1H-indol-1-yl)-2-fluoro-6- |
| | (tetrahydrofuran-3-ylamino)benzamide; |
| 766 | 2-(4,4-difluorocyclohexylamino)-6-fluoro-4-(3,6,6-trimethyl-4-oxo-4,5,6,7-tetrahydro- |
| | 1H-indol-1-yl)benzamide; |
| 767 | 2-fluoro-6-(2-hydroxycyclopentylamino)-4-(3,6,6-trimethyl-4-oxo-4,5,6,7-tetrahydro-1H- |
| | indazol-1-yl)benzamide; |
| 768 | 2-(cyclopentylamino)-6-fluoro-4-(3,6,6-trimethyl-4-oxo-4,5,6,7-tetrahydro-1H-indol-1- |
| | yl)benzamide; |
| 769 | 2-(2-carbamoyl-3-fluoro-5-(3,6,6-trimethyl-4-oxo-4,5,6,7-tetrahydro-1H-indazol-1- |
| | yl)phenylamino)cyclopentyl 2-aminoacetate; |
| 770 | 2-(cyclopentylamino)-4-(6,6-dimethyl-4-oxo-3-(trifluoromethyl)-4,5,6,7-tetrahydro-1H- |
| | indol-1-yl)-6-fluorobenzamide; |
| 771 | 4-(2-carbamoyl-5-(6,6-dimethyl-4-oxo-3-(trifluoromethyl)-4,5,6,7-tetrahydro-1H-indol-1- |
| | yl)-3-fluorophenylamino)cyclohexyl 2-aminoacetate hydrochloride; |
| 772 | 2-(4,4-difluorocyclohexylamino)-4-(6,6-dimethyl-4-oxo-3-(trifluoromethyl)-4,5,6,7- |
| | tetrahydro-1H-indol-1-yl)-6-fluorobenzamide; |
| 773 | 2-(cyclohexylamino)-4-(6,6-dimethyl-4-oxo-3-(trifluoromethyl)-4,5,6,7-tetrahydro-1H- |
| | indol-1-yl)-6-fluorobenzamide; |
| 774 | 2-(2-carbamoyl-5-(3-ethyl-6,6-dimethyl-4-oxo-4,5,6,7-tetrahydro-1H-indazol-1- |
| | yl)phenylamino)cyclohexyl 2-aminoacetate; |
| 775 | 4-(3-(difluoromethyl)-6,6-dimethyl-4-oxo-4,5,6,7-tetrahydro-1H-indazol-1-yl)-2-(2,2,6,6- |
| | tetramethylpiperidin-4-ylamino)benzamide; |
| 776 | 2-(2-carbamoyl-5-(6,6-dimethyl-4-oxo-3-(trifluoromethyl)-4,5,6,7-tetrahydro-1H- |
| | indazol-1-yl)phenylamino)cyclopentyl 2-aminoacetate; |
| 777 | 2-(2-carbamoyl-5-(6,6-dimethyl-4-oxo-3-(trifluoromethyl)-4,5,6,7-tetrahydro-1H- |
| | indazol-1-yl)phenylamino)cyclohexyl 2-aminoacetate; |
| 778 | 2-(2-carbamoyl-5-(6,6-dimethyl-4-oxo-3-(trifluoromethyl)-4,5,6,7-tetrahydro-1H-indol-1- |
| | yl)phenylamino)cyclopentyl 2-aminoacetate methanesulfonate; |
| 779 | 2-(2-carbamoyl-5-(6,6-dimethyl-4-oxo-3-(trifluoromethyl)-4,5,6,7-tetrahydro-1H- |
| | indazol-1-yl)phenylamino)cyclopentyl 1-aminocyclopropanecarboxylate |
| | methanesulfonate; |
| 780 | 4-(2-carbamoyl-5-(6,6-dimethyl-4-oxo-3-(trifluoromethyl)-4,5,6,7-tetrahydro-1H-indol-1- |
| | yl)phenylamino)cyclohexyl 2-aminoacetate methanesulfonate; |
| 781 | 4-(2-carbamoyl-5-(6,6-dimethyl-4-oxo-3-(trifluoromethyl)-4,5,6,7-tetrahydro-1H- |
| | indazol-1-yl)phenylamino)cyclohexyl 2-(2-aminoacetamido)acetate 2,2,2- |
| | trifluoroacetate; |
| 782 | 4-(3-ethyl-6,6-dimethyl-4-oxo-4,5,6,7-tetrahydro-1H-indazol-1-yl)-2-(2- |
| | hydroxycyclopentylamino)-6-methoxybenzamide; |
| 783 | 2-(oxetan-3-ylamino)-4-(3,6,6-trimethyl-4-oxo-4,5,6,7-tetrahydro-1H-indazol-1- |
| | yl)benzamide; |
| 784 | 4-(6,6-dimethyl-4-oxo-3-(trifluoromethyl)-4,5,6,7-tetrahydro-1H-indazol-1-yl)-2- |
| | (tetrahydrofuran-3-ylamino)benzamide; |
| 785 | 4-(6-ethyl-3,6-dimethyl-4-oxo-4,5,6,7-tetrahydro-1H-indazol-1-yl)-2-(4- |
| | hydroxycyclohexylamino)benzamide; |
| 786 | 4-(6,6-dimethyl-4-oxo-3-(trifluoromethyl)-4,5,6,7-tetrahydro-1H-indol-1-yl)-2-(2- |
| | hydroxycyclopentylamino)benzamide; |
| 787 | 4-(3-ethyl-6,6-dimethyl-4-oxo-4,5,6,7-tetrahydro-1H-indol-1-yl)-2-(tetrahydrofuran-3- |
| | ylamino)benzamide; |
| 788 | 4-(3-ethyl-6,6-dimethyl-4-oxo-4,5,6,7-tetrahydro-1H-indazol-1-yl)-2-methoxy-6- |
| | (tetrahydro-2H-pyran-4-ylamino)benzamide; |
| 789 | 4-(6,6-dimethyl-4-oxo-3-(2,2,2-trifluoroethyl)-4,5,6,7-tetrahydro-1H-indazol-1-yl)-2-(4- |
| | hydroxycyclohexylamino)benzamide; |
| 790 | 4-(6,6-dimethyl-4-oxo-3-(trifluoromethyl)-4,5,6,7-tetrahydro-1H-indazol-1-yl)-2-(2- |
| | hydroxycyclopentylamino)benzamide; |
| 791 | 2-(cyclopentylamino)-4-(6,6-dimethyl-4-oxo-3-(trifluoromethyl)-4,5,6,7-tetrahydro-1H- |
| | indazol-1-yl)benzamide; |
| 792 | 2-(tetrahydrofuran-3-ylamino)-4-(3,6,6-trimethyl-4-oxo-4,5,6,7-tetrahydro-1H-indol-1- |
| | yl)benzamide; |
| 793 | 4-(6,6-dimethyl-4-oxo-3-(trifluoromethyl)-4,5,6,7-tetrahydro-1H-indol-1-yl)-2- |
| | (tetrahydro-2H-pyran-4-ylamino)benzamide; |
| 794 | 4-(6,6-dimethyl-4-oxo-3-(trifluoromethyl)-4,5,6,7-tetrahydro-1H-indol-1-yl)-2- |
| | (tetrahydrofuran-3-ylamino)benzamide; |
| 795 | 2-(cyclopentylamino)-4-(3,6,6-trimethyl-4-oxo-4,5,6,7-tetrahydro-1H-indol-1- |
| | yl)benzamide; |
| 796 | 4-(6,6-dimethyl-4-oxo-3-(trifluoromethyl)-4,5,6,7-tetrahydro-1H-indol-1-yl)-2-(4- |
| | hydroxycyclohexylamino)benzamide; |
| 797 | 2-(cyclopentylamino)-4-(6,6-dimethyl-4-oxo-3-(trifluoromethyl)-4,5,6,7-tetrahydro-1H- |
| | indol-1-yl)benzamide; |
| 798 | 2-(4,4-difluorocyclohexylamino)-4-(3,6,6-trimethyl-4-oxo-4,5,6,7-tetrahydro-1H-indol-1- |
| | yl)benzamide; |
| 799 | 2-(4,4-difluorocyclohexylamino)-4-(3,6,6-trimethyl-4-oxo-4,5,6,7-tetrahydro-1H- |
| | indazol-1-yl)benzamide; |
| 800 | 3-(4,4-difluorocyclohexylamino)-5-(3,6,6-trimethyl-4-oxo-4,5,6,7-tetrahydro-1H-indol-1- |
| | yl)picolinamide; |
| 801 | 3-(2-hydroxycyclohexylamino)-5-(3,6,6-trimethyl-4-oxo-4,5,6,7-tetrahydro-1H-indol-1- |
| | yl)picolinamide; |
| 802 | 5-(6,6-dimethyl-4-oxo-3-(trifluoromethyl)-4,5,6,7-tetrahydro-1H-indol-1-yl)-3-(2- |
| | hydroxycyclohexylamino)picolinamide; |
| 803 | 3-(4,4-difluorocyclohexylamino)-5-(6,6-dimethyl-4-oxo-3-(trifluoromethyl)-4,5,6,7- |
| | tetrahydro-1H-indol-1-yl)picolinamide; |
| 804 | 2-(cyclohexylamino)-4-(3,6,6-trimethyl-4-oxo-4,5,6,7-tetrahydro-1H-indazol-1- |
| | yl)benzamide; |
| 805 | 2-(4,4-difluorocyclohexylamino)-4-(6,6-dimethyl-4-oxo-3-(trifluoromethyl)-4,5,6,7- |
| | tetrahydro-1H-indol-1-yl)benzamide; |
| 806 | 4-(2-carbamoyl-3-fluoro-5-(3,6,6-trimethyl-4-oxo-4,5,6,7-tetrahydro-1H-indazol-1- |
| | yl)phenylamino)cyclohexyl 2-(tert-butoxycarbonylamino)acetate; |
| 807 | 2-(2-carbamoyl-5-(6,6-dimethyl-4-oxo-3-(trifluoromethyl)-4,5,6,7-tetrahydro-1H- |
| | indazol-1-yl)phenylamino)cyclopentyl 2-(tert-butoxycarbonylamino)acetate; |
| 808 | 2-(cyclopentylamino)-4-(3,6,6-trimethyl-4-oxo-4,5,6,7-tetrahydro-1H-indazol-1- |
| | yl)benzamide; |
| 809 | 2-(cycloheptylamino)-4-(3,6,6-trimethyl-4-oxo-4,5,6,7-tetrahydro-1H-indazol-1- |
| | yl)benzamide; |
| 810 | 5-(6,6-dimethyl-4-oxo-3-(trifluoromethyl)-4,5,6,7-tetrahydro-1H-indazol-1-yl)-3-((trans)- |
| | 4-hydroxycyclohexylamino)picolinamide; |
| 811 | trans-4-(2-carbamoyl-5-(6,6-dimethyl-4-oxo-3-(trifluoromethyl)-4,5,6,7-tetrahydro-1H- |
| | indazol-1-yl)phenylamino)cyclohexyl 3-aminopropanoate; |
| 812 | (S)-(trans-4-(2-carbamoyl-5-(6,6-dimethyl-4-oxo-3-(trifluoromethyl)-4,5,6,7-tetrahydro- |
| | 1H-indazol-1-yl)phenylamino)cyclohexyl) 2,6-bis(tert-butoxycarbonylamino)hexanoate; |
| 813 | (S)-(trans-4-(2-carbamoyl-5-(6,6-dimethyl-4-oxo-3-(trifluoromethyl)-4,5,6,7-tetrahydro- |
| | 1H-indazol-1-yl)phenylamino)cyclohexyl) 2,6-diaminohexanoate; |
| 814 | 4-(2-carbamoyl-5-(3,6,6-trimethyl-4-oxo-4,5,6,7-tetrahydro-1H-indazol-1- |
| | yl)phenylamino)cyclohexyl 3-(tert-butoxycarbonylamino)propanoate; |
| 815 | 4-(2-carbamoyl-5-(3,6,6-trimethyl-4-oxo-4,5,6,7-tetrahydro-1H-indazol-1- |
| | yl)phenylamino)cyclohexyl 3-aminopropanoate; |
| 816 | (S)-4-(2-carbamoyl-5-(6,6-dimethyl-4-oxo-3-(trifluoromethyl)-4,5,6,7-tetrahydro-1H- |
| | indazol-1-yl)phenylamino)cyclohexyl 3-(2,5-bis(tert- |
| | butoxycarbonylamino)pentanamido)propanoate; and |
| 817 | (S)-4-(2-carbamoyl-5-(6,6-dimethyl-4-oxo-3-(trifluoromethyl)-4,5,6,7-tetrahydro-1H- |
| | indazol-1-yl)phenylamino)cyclohexyl 3-(2,5-diaminopentanamido)propanoate; |
| 818 | trans-4-(2-carbamoyl-5-(3,6,6-trimethyl-4-oxo-4,5,6,7-tetrahydro-1H-indol-1- |
| | yl)phenylamino)cyclohexyl 2-(3-aminopropanamido)acetate; |
| 819 | trans-4-(2-carbamoyl-5-(6,6-dimethyl-4-oxo-3-(trifluoromethyl)-4,5,6,7-tetrahydro-1H- |
| | indazol-1-yl)phenylamino)cyclohexyl 2-(3-aminopropanamido)acetate; |
| 820 | trans-4-(2-carbamoyl-5-(3-ethyl-6,6-dimethyl-4-oxo-4,5,6,7-tetrahydro-1H-indazol-1- |
| | yl)phenylamino)cyclohexyl 2-(3-aminopropanamido)acetate; |
| 821 | trans-4-(2-carbamoyl-5-(3-(cyclopropylmethyl)-6,6-dimethyl-4-oxo-4,5,6,7-tetrahydro- |
| | 1H-indazol-1-yl)phenylamino)cyclohexyl 2-(3-aminopropanamido)acetate; |
| 822 | trans-4-(2-carbamoyl-5-(3,6,6-trimethyl-4-oxo-4,5,6,7-tetrahydro-1H-indazol-1- |
| | yl)phenylamino)cyclohexyl 2-(3-aminopropanamido)acetate; |
| 823 | trans-4-(2-carbamoyl-5-(2,3,6,6-tetramethyl-4-oxo-4,5,6,7-tetrahydro-1H-indol-1- |
| | yl)phenylamino)cyclohexyl 2-(3-aminopropanamido)acetate; |
| 824 | (S)-(trans-4-(2-carbamoyl-5-(3,6,6-trimethyl-4-oxo-4,5,6,7-tetrahydro-1H-indol-1- |
| | yl)phenylamino)cyclohexyl) 2-(3-aminopropanamido)propanoate; |
| 825 | (S)-(trans-4-(2-carbamoyl-5-(6,6-dimethyl-4-oxo-3-(trifluoromethyl)-4,5,6,7-tetrahydro- |
| | 1H-indazol-1-yl)phenylamino)cyclohexyl) 2-(3-aminopropanamido)propanoate; |
| 826 | (S)-(trans-4-(2-carbamoyl-5-(3-ethyl-6,6-dimethyl-4-oxo-4,5,6,7-tetrahydro-1H-indazol- |
| | 1-yl)phenylamino)cyclohexyl) 2-(3-aminopropanamido)propanoate; |
| 827 | (S)-(trans-4-(2-carbamoyl-5-(3-(cyclopropylmethyl)-6,6-dimethyl-4-oxo-4,5,6,7- |
| | tetrahydro-1H-indazol-1-yl)phenylamino)cyclohexyl) 2-(3-aminopropanamido)-3- |
| | methylbutanoate; |
| 828 | trans-4-(2-carbamoyl-5-(3,6,6-trimethyl-4-oxo-4,5,6,7-tetrahydro-1H-indazol-1- |
| | yl)phenylamino)cyclohexyl 2-(3-aminopropanamido)acetate; |
| 829 | (S)-(trans-4-(2-carbamoyl-5-(2,3,6,6-tetramethyl-4-oxo-4,5,6,7-tetrahydro-1H-indol-1- |
| | yl)phenylamino)cyclohexyl) 2-(3-aminopropanamido)-3-methylbutanoate; |
| 830 | trans-4-(2-carbamoyl-5-(3,6,6-trimethyl-4-oxo-4,5,6,7-tetrahydro-1H-indol-1- |
| | yl)phenylamino)cyclohexyl 3-aminopropanoate; |
| 831 | trans-4-(2-carbamoyl-5-(3-ethyl-6,6-dimethyl-4-oxo-4,5,6,7-tetrahydro-1H-indazol-1- |
| | yl)phenylamino)cyclohexyl 3-aminopropanoate; |
| 832 | trans-4-(2-carbamoyl-5-(3-(cyclopropylmethyl)-6,6-dimethyl-4-oxo-4,5,6,7-tetrahydro- |
| | 1H-indazol-1-yl)phenylamino)cyclohexyl 3-aminopropanoate; |
| 833 | trans-4-(2-carbamoyl-5-(2,3,6,6-tetramethyl-4-oxo-4,5,6,7-tetrahydro-1H-indol-1- |
| | yl)phenylamino)cyclohexyl 3-aminopropanoate; |
| 834 | sodium trans-4-(2-carbamoyl-5-(2,3,6,6-tetramethyl-4-oxo-4,5,6,7-tetrahydro-1H-indol- |
| | 1-yl)phenylamino)cyclohexyl hydrogenphosphate; |
| 835 | sodium trans-4-(2-carbamoyl-5-(6,6-dimethyl-4-oxo-3-(trifluoromethyl)-4,5,6,7- |
| | tetrahydro-1H-indazol-1-yl)phenylamino)cyclohexyl hydrogenphosphate; |
| 836 | sodium trans-4-(2-carbamoyl-5-(3,6,6-trimethyl-4-oxo-4,5,6,7-tetrahydro-1H-indol-1- |
| | yl)phenylamino)cyclohexyl hydrogenphosphate; |
| 837 | sodium trans-4-(2-carbamoyl-5-(3-ethyl-6,6-dimethyl-4-oxo-4,5,6,7-tetrahydro-1H- |
| | indazol-1-yl)phenylamino)cyclohexyl hydrogenphosphate; |
| 838 | sodium trans-4-(2-carbamoyl-5-(3,6,6-trimethyl-4-oxo-4,5,6,7-tetrahydro-1H-indazol-1- |
| | yl)phenylamino)cyclohexyl hydrogenphosphate; |
| 839 | sodium trans-4-(2-carbamoyl-5-(3-(cyclopropylmethyl)-6,6-dimethyl-4-oxo-4,5,6,7- |
| | tetrahydro-1H-indazol-1-yl)phenylamino)cyclohexyl hydrogenphosphate; |
| 840 | sodium trans-4-(2-carbamoyl-5-(3,6,6-trimethyl-4-oxo-4,5,6,7-tetrahydro-1H-indol-1- |
| | yl)phenylamino)cyclohexyl sulfate; |
| 841 | sodium trans-4-(2-carbamoyl-5-(3-ethyl-6,6-dimethyl-4-oxo-4,5,6,7-tetrahydro-1H- |
| | indazol-1-yl)phenylamino)cyclohexyl sulfate; |
| 842 | sodium trans-4-(2-carbamoyl-5-(6,6-dimethyl-4-oxo-3-(trifluoromethyl)-4,5,6,7- |
| | tetrahydro-1H-indazol-1-yl)phenylamino)cyclohexyl sulfate; and |
| 843 | sodium trans-4-(2-carbamoyl-5-(3-(cyclopropylmethyl)-6,6-dimethyl-4-oxo-4,5,6,7- |
| | tetrahydro-1H-indazol-1-yl)phenylamino)cyclohexyl sulfate. |
| 844 | 4-(6,6-dimethyl-4-oxo-3-trifluoromethyl-4,5,6,7-tetrahydro-indazol-1-yl)-2-(4-hydroxy- |
| | cyclohexylamino)-benzamide; |
| 845 | 4-({2-(aminocarbonyl)-5-[6,6-dimethyl-4-oxo-3-(trifluoromethyl)-4,5,6,7-tetrahydro-1H- |
| | indazol-1-yl]phenyl}amino)cyclohexyl glycinate. |

The synthesis and characterization data of the above-listed compounds is described in U.S. Pat. No. 7,358,370. Particular compounds suitable for use in the methods described herein include:
4-(6,6-dimethyl-4-oxo-3-trifluoromethyl-4,5,6,7-tetrahydro-indazol-1-yl)-2-(trans-4-hydroxy-cyclohexylamino)-benzamide;
trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate;
2-((4-hydroxycyclohexyl)amino)-4-(3,6,6-trimethyl-4-oxo-4,5,6,7-tetrahydro-1H-indazol-1-yl)benzamide;
2-((2-hydroxyethyl)amino)-4-(3,6,6-trimethyl-4-oxo-4,5,6,7-tetrahydro-1H-indazol-1-yl)benzamide;
2-((2-methoxyethyl)amino)-4-(3,6,6-trimethyl-4-oxo-4,5,6,7-tetrahydro-1H-indazol-1-yl)benzamide;
2-((2-ethoxyethyl)amino)-4-(3,6,6-trimethyl-4-oxo-4,5,6,7-tetrahydro-1H-indazol-1-yl)benzamide;
2-((2-(2-(2-aminoethoxy)ethoxy)ethyl)amino)-4-(3,6,6-trimethyl-4-oxo-4,5,6,7-tetrahydro-1H-indazol-1-yl)benzamide;
2-((3-hydroxypropyl)amino)-4-(3,6,6-trimethyl-4-oxo-4,5,6,7-tetrahydro-1H-indazol-1-yl)benzamide;
2-((3-methoxypropyl)amino)-4-(3,6,6-trimethyl-4-oxo-4,5,6,7-tetrahydro-1H-indazol-1-yl)benzamide; and
2-((3-ethoxypropyl)amino)-4-(3,6,6-trimethyl-4-oxo-4,5,6,7-tetrahydro-1H-indazol-1-yl)benzamide.

In some embodiments of the methods, the Hsp90 inhibitor is a compound listed in Table 1, or a pharmaceutically acceptable salt thereof. In other embodiments of the methods, the Hsp90 inhibitor is a compound of formula (I):

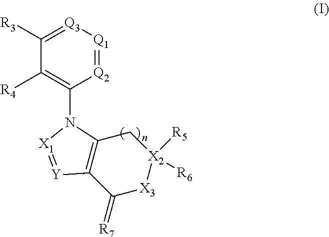

or a pharmaceutically acceptable salt thereof.

### Pharmaceutical Compositions

In some embodiments, the method comprises the administration of the Hsp90 inhibitor in a pharmaceutical composition having at least one pharmaceutically acceptable carrier, solvent, adjuvant or diluent.

The compounds described herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions described herein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions disclosed herein may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The compositions disclosed herein may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

The compounds of the present invention may be administered alone or in combination with at least one antiviral agent. Examples include, but are not limited to, fusion inhibitors (such as Maraviroc and Enfuvirtide), nucleoside reverse transcriptase inhibitors (NRTI) and nucleotide reverse transcriptase inhibitors (NtRTI) (such zidovudine, abacavir, lamivudine, emtricitabine, and tenofoviras), non-nucleoside reverse transcriptase inhibitors (NNRTI) (such as nevirapine, efavirenz, etravirine and rilpivirine), integrase inhibitors (such as elvitegravir and dolutegravir), and protease inhibitors (such as Lopinavir, Indinavir, Nelfinavir, Amprenavir, Ritonavir, Darunavir and atazanavir). In certain embodiments, the antiviral agent is selected from the group consisting of the nucleoside reverse transcriptase inhibitors, the non-nucleoside reverse transcriptase inhibitors and the protease inhibitors. The compounds of the present invention may be combined with one or more antiviral agents simultaneously or sequentially.

### DEFINITIONS

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designated number of carbon atoms. Alkyl groups may be straight, or branched. Examples of alkyl include methyl, ethyl, propyl, isopropyl, butyl, iso-, sec- and tert-butyl, pentyl, hexyl, heptyl, 3-ethylbutyl, and the like.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenoxy” refers to an alkenyl group attached to the parent group through an oxygen atom.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl. The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl, mono- and di(C₁-C₈alkyl)amino(C₁-C₈)alkyl, C₁-C₈acyl, C₁-C₈acyloxy, C₁-C₈sulfonyl, C₁-C₈thio, C₁-C₈sulfonamido, C₁-C₈aminosulfonyl.

The term “carboxy” as used herein, means a —CO₂H group.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃. A preferred haloalkoxy group is trifluoromethoxy.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

As used here, the terms “treatment” and “treating” means:
(i) inhibiting the progression the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

### Examples

### Example 1

### Evaluation of HIV-1_{Ba-L} Replication in Peripheral Blood Mononuclear Cells (PBMCs)

Zidovudine (or azidothymidine or AZT), and 4-(6,6-Dimethyl-4-oxo-3-trifluoromethyl-4,5,6,7-tetrahydro-indazol-1-yl)-2-(trans-4-hydroxy-cyclohexylamino)-benzamide, (Compound 9) were evaluated for HIV-1Ba-L replication in peripheral blood mononuclear cells (PBMCs).

Anti-HIV Efficacy Evaluation in Fresh Human PBMCs Assay Protocol:

Fresh human PBMCs, seronegative for HIV and HBV, are isolated from screened donors (Biological Specialty Corporation, Colmar, Pa.). Cells are pelleted/washed 2-3 times by low speed centrifugation and re-suspension in PBS to remove contaminating platelets. The Leukophoresed blood is then diluted 1:1 with Dulbecco's Phosphate Buffered Saline (DPBS) and layered over 14 mL of Lymphocyte Separation Medium (LSM; Cellgro® by Mediatech, Inc.; density 1.078+/−0.002 g/ml; Cat.#85-072-CL) in a 50 mL centrifuge tube and then centrifuged for 30 minutes at 600×g. Banded PBMCs are gently aspirated from the resulting interface and subsequently washed 2× with PBS by low speed centrifugation. After the final wash, cells are enumerated by trypan blue exclusion and re-suspended at 1×10⁶ cells/mL in RPMI 1640 supplemented with 15% Fetal Bovine Serum (FBS), and 2 mM L-glutamine, 4 lug/mL Phytohemagglutinin (PHA, Sigma). The cells are allowed to incubate for 48-72 hours at 37° C. After incubation, PBMCs are centrifuged and re-suspended in RPMI 1640 with 15% FBS, 2 mM L-glutamine, 100 U/mL penicillin, 100 pg/mL streptomycin, and 20 U/mL recombinant human IL-2 (R&D Systems, Inc). IL-2 is included in the culture medium to maintain the cell division initiated by the PHA mitogenic stimulation. PBMCs are maintained in this medium at a concentration of 1-2×10⁶ cells/mL with biweekly medium changes until used in the assay protocol. Cells are kept in culture for a maximum of two weeks before being deemed too old for use in assays and discarded. MDMs are depleted from the culture as the result of adherence to the tissue culture flask.

For the standard PBMC assay, PHA stimulated cells from at least two normal donors are pooled (mixed together), diluted in fresh medium to a final concentration of 1×10⁶ cells/mL, and plated in the interior wells of a 96 well round bottom microplate at 50 uL/well (5×10⁴ cells/well) in a standard format developed by the Infectious Disease Research department of Southern Research Institute. Pooling (mixing) of mononuclear cells from more than one donor is used to minimize the variability observed between individual donors, which results from quantitative and qualitative differences in HIV infection and overall response to the PHA and IL-2 of primary lymphocyte populations. Each plate contains virus/cell control wells (cells plus virus), experimental wells (drug plus cells plus virus) and compound control wells (drug plus media without cells, necessary for MTS monitoring of cytotoxicity). In this in vitro assay, PBMC viability remains high throughout the duration of the incubation period. Therefore, infected wells are used in the assessment of both antiviral activity and cytotoxicity. Test drug dilutions are prepared at a 2× concentration in microtiter tubes and 100 μL of each concentration (nine total concentrations) are placed in appropriate wells using the standard format. 50 μL of a predetermined dilution of virus stock is placed in each test well (final MOI˜=0.1). The PBMC cultures are maintained for seven days following infection at 37° C., 5% CO₂. After this period, cell-free supernatant samples are collected for analysis of reverse transcriptase activity and/or p24 antigen content. Following removal of supernatant samples, compound cytotoxicity is measured by addition of MTS to the plates for determination of cell viability. Wells are also examined microscopically and any abnormalities are noted.

Reverse Transcriptase Activity Assay Protocol:

A microtiter plate-based reverse transcriptase (RT) reaction is utilized (Buckheit et al., AIDS Research and Human Retroviruses 7:295-302, 1991). Tritiated thymidine triphosphate (³H-TTP, 80 Ci/mmol, NEN) is received in 1:1 dH₂O:Ethanol at 1 mCi/mL. Poly rA:oligo dT template:primer (GE Healthcare) is prepared as a stock solution by combining 150 μL poly rA (20 mg/mL) with 0.5 mL oligo dT (20 units/mL) and 5.35 mL sterile dH₂O followed by aliquoting (1.0 mL) and storage at −20° C. The RT reaction buffer is prepared fresh on a daily basis and consists of 125 μL 1.0 M EGTA, 125 μL dH₂O, 125 μL 20% Triton X100, 50 μL 1.0 M Tris (pH 7.4), 50 μL 1.0 M DTT, and 40 μL 1.0 M MgCl₂. The final reaction mixture is prepared by combining 1 part ³H-TTP, 4 parts dH₂O, 2.5 parts poly rA:oligo dT stock and 2.5 parts reaction buffer. Ten microliters of this reaction mixture is placed in a round bottom microtiter plate and 15 μL of virus containing supernatant is added and mixed. The plate is incubated at 37° C. for 60 minutes. Following incubation, the reaction volume is spotted onto DE81 filter-mats (Wallac), washed 5 times for 5 minutes each in a 5% sodium phosphate buffer or 2×SSC (Life Technologies). Next they are washed 2 times for 1 minute each in distilled water, 2 times for 1 minute each in 70% ethanol, and then dried. Incorporated radioactivity (counts per minute, CPM) is quantified using standard liquid scintillation techniques.

MTS Staining for PBMC Viability to Measure Cytotoxicity Protocol:

At assay termination, assay plates are stained with the soluble tetrazolium-based dye MTS (CellTiter 96 Reagent, Promega) to determine cell viability and quantify compound toxicity. The mitochondrial enzymes of metabolically active cells metabolize MTS to yield a soluble formazan product. This allows the rapid quantitative analysis of cell viability and compound cytotoxicity. The MTS is a stable solution that does not require preparation before use. At termination of the assay, 20 uL of MTS reagent is added per well. The microtiter plates are then incubated 4-6 hrs at 37° C. The incubation intervals were chosen based on empirically determined times for optimal dye reduction. Adhesive plate sealers are used in place of the lids, the sealed plate is inverted several times to mix the soluble formazan product and the plate is read spectrophotometrically at 490/650 nm with a Molecular Devices Vmax or SpectraMaxPlus plate reader.

IC₅₀ (50% inhibition of virus replication), IC₉₀ (90% inhibition of virus replication), IC₉₅ (95% inhibition of virus replication), TC₅₀ (50% cytotoxicity), TC₉₀ (90% cytotoxicity), TC₉₅ (95% cytotoxicity) and therapeutic index values (TI=TC/IC; also referred to as Antiviral Index or AI) were calculated using a standard computing program. The results are show in Table 2.

TABLE 2
Evaluation of compounds vs. HIV-1_{Ba-L} in PBMCs

| | | Antiviral | | Selectivity |
|---|---|---|---|---|
| | High-test | Activity | Cytotoxicity | Index |
| Comp. | Concentration | IC₅₀ (nM) | TC₅₀ (nM) | TI (TC₅₀/IC₅₀) |
| AZT | 1,000 nM | 40.5 | >1,000 | >24.7 |
| 9 | 2 μM | 20 | 30 | 1.5 |

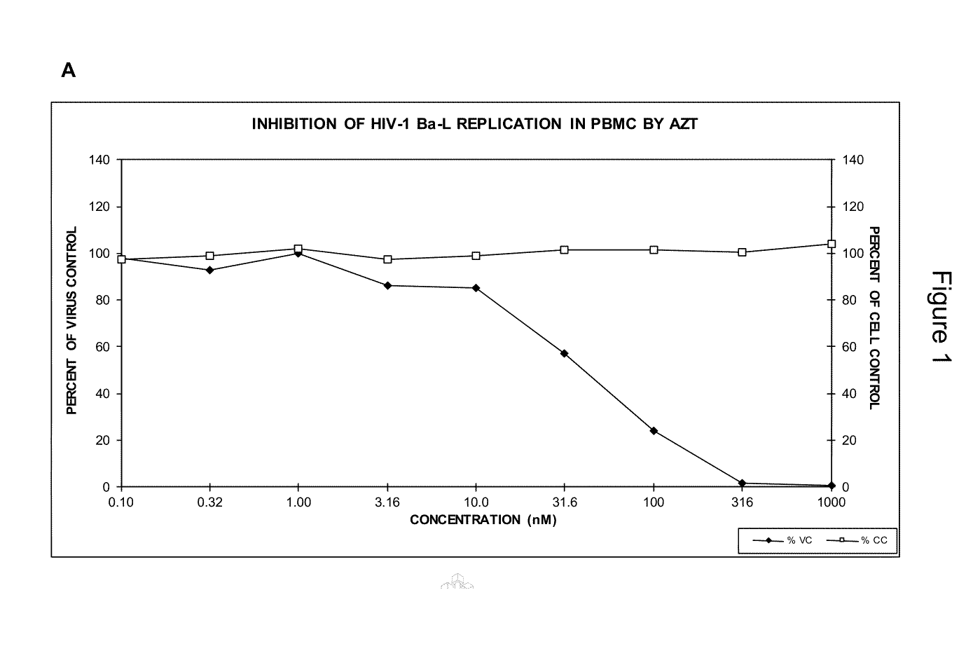

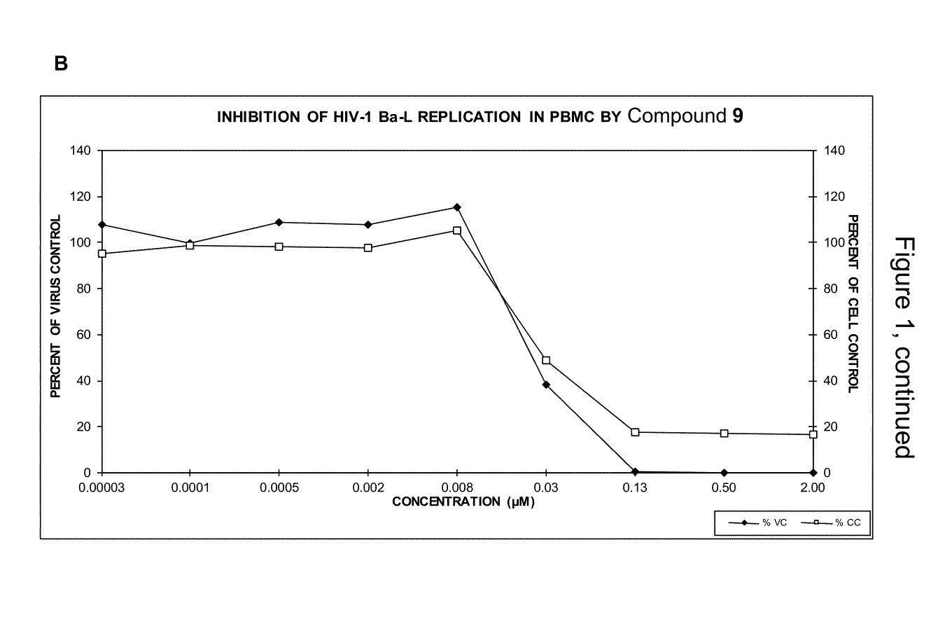

Dose dependent evaluation of virus control vs. cell control is also illustrated in FIG. 1 for each compound. The MTS assay utilizes metabolic activity rather than cell death as an endpoint in measuring cytotoxicity. The MTS cell proliferation assay method in PBMCs showed TC₅₀ and TC₉₀ values of 30 nM and >2000 nM, respectively. This resulted in calculated antiviral indices at 50% inhibition of 1.5 and >22.1 at 90% inhibition. Table 3 provides antiviral index for each compound.

TABLE 3
Evaluation of compounds vs. HIV-1_{Ba-L} in PBMCs

| Comp. | | 50% | 90% | 95% |
|---|---|---|---|---|
| AZT | TC (nM) | >1000 | >1000 | >1000 |
| | IC (nM) | 40.5 | 208 | 269 |
| | Selectivity index | >24.7 | >4.81 | >3.71 |
| | (TI) | | | |
| 9 | TC (nM) | 30 | >2000 | >2000 |
| | IC (nM) | 20 | 90 | 11 |
| | Selectivity index | 1.5 | >22.1 | >18.2 |
| | (TI) | | | |

Plasma levels obtained from human studies, as discussed below are consistent with data provided in Table 2 and 3.

### Example 2

### Evaluation of Plasma Level Concentrations in Humans

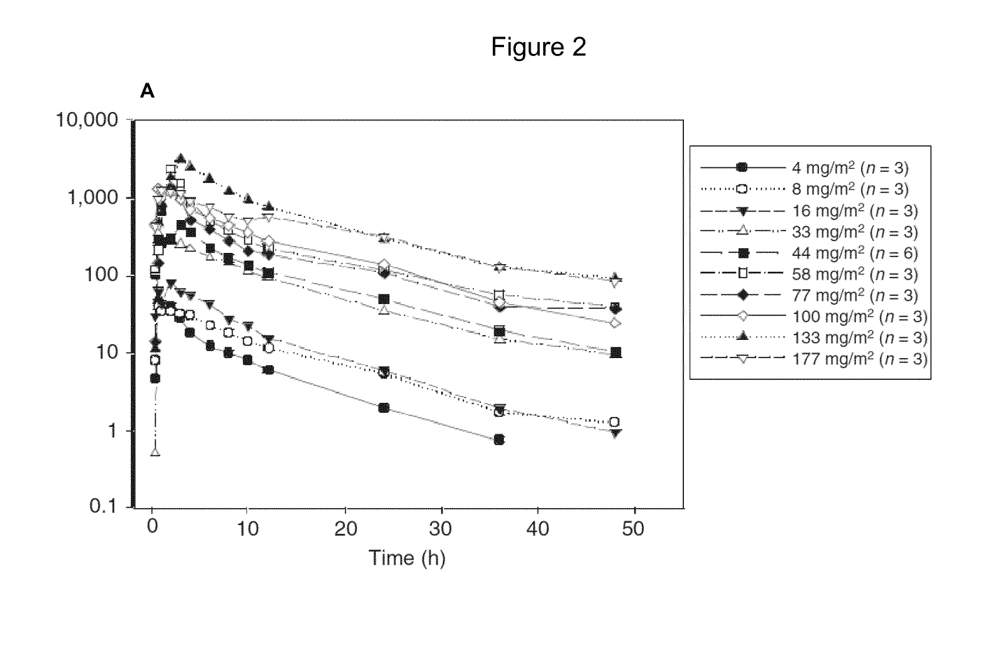

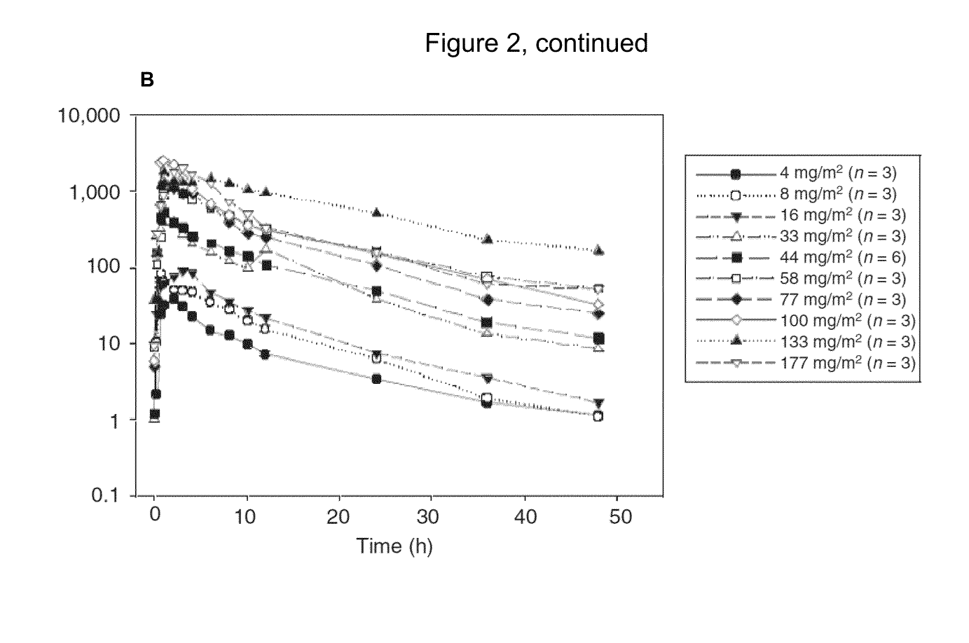

Blood was drawn on day 1 and days 15/18 of cycle 1 at the following time points: pre-dose and post-dose at 20 and 40 minutes and at 1, 2, 3, 4, 6, 8, 10, 12, 24, 36, and 48 hours. During subsequent cycles, blood was drawn before dosing on day 1 only. Samples were analyzed by a validated liquid chromatography/tandem mass spectrometry (LC/MS-MS) method developed at PPD Development, Richmond, Va. Mean plasma concentration-time profile for escalating dose levels of Compound 9 after first dose (semilog scale) on (A) cycle 1, day 1 and (B) cycle 1, days 15/18 (error bars are not shown for clarity). Results are illustrated in FIGS. 2A and 2B, which show mean plasma concentration-time profile for escalating dose levels of Compound 9 after first dose (semi-log scale) on (2A) cycle 1, day 1 and (2B) cycle 1, days 15/18. Additional discussion is provided by Rajan A, et al. 2011, Clin Cancer Res. 17: 6831-6839, which is incorporated by reference herein.

### Example 3

### Evaluation of Hepatitis B Virus (HBV) Activity in Human Hepatoma Cells Transfected with HBV DNA (HepG2 2.2.15 Cell Line)

Lamivudine (2′,3′-dideoxy-3′-thiacytidine, or 3TC), and 4-(6,6-Dimethyl-4-oxo-3-trifluoromethyl-4,5,6,7-tetrahydro-indazol-1-yl)-2-(trans-4-hydroxy-cyclohexyl-amino)-benzamide, (Compound 9) were evaluated for HBV activity in human hepatoma cells transfected with HBV DNA (HepG2 2.2.15 cell line).

The anti-HBV assay employed real-time qPCR (TaqMan) to directly measure extracellular HBV DNA copy number. HepG2-2.2.15 cells were plated in 96-well microtiter plates. Only the interior wells were utilized to reduce “edge effects” observed during cell culture; the exterior wells were filled with complete medium to help minimize sample evaporation. After 16-24 hours the confluent monolayer of HepG2-2.2.15 cells was washed and the medium replaced with complete medium containing various concentrations of a test compound in triplicate. 3TC was used as positive control, while media alone was added to cells as negative control (virus control, VC). Three days later the culture medium was replaced with fresh medium containing the appropriately diluted drug. Six days following the initial administration of the test compound, the cell culture supernatant collected, treated with pronase and then used in a real-time quantitative TaqMan qPCR assay. The PCR-amplified HBV DNA was detected in real-time by monitoring increases in fluorescence signals that result from the exonucleolytic degradation of a quenched fluorescent probe molecule that hybridizes to the amplified HBV DNA. For each PCR amplification, a standard curve was simultaneously generated using dilutions of purified HBV DNA. Antiviral activity was calculated from the reduction in HBV DNA levels (IC₅₀). CellTiter 96® AQueous One Solution Cell Proliferation Assay kit (Promega) was employed to measure cell viability which was used to calculate cytotoxicity (TC₅₀). The therapeutic index (TI) was calculated as TC₅₀/IC₅₀. The results are show in Table 4.

TABLE 4
Evaluation of compounds vs. HBV in HepG2 2.2.15

| | | Antiviral | | Selectivity |
|---|---|---|---|---|
| | High-test | Activity | Cytotoxicity | Index |
| Comp. | Concentration | IC₅₀ (nM) | TC₅₀ (nM) | TI (TC₅₀/IC₅₀) |
| 3TC | 500 nM | 35.6 | >500 | >14.0 |
| 9 | 2 μM | 600 | 73 | 0.12 |

An IC₅₀ value of 600 nM indicates that Compound 9 is inactive against hepatitis B virus. Inactivity of Compound 9 against hepatitis B virus was confirmed because an IC₉₀ could not be determined (>2000 nM).

### Example 4

### Evaluation of SIV Virus Inhibition

Non-human primate (NHP) cells were activated in IL-2 growth medium (GM) containing PHA-P at a concentration of 10 μg/ml for 24 hrs. The next day cells were washed and re-suspended in fresh IL-2 GM. Compound 9 was serially diluted at concentrations ranging from 0.0005 to 50 μM in a 96 well u-bottom plate containing IL-2 GM. Activated cells were then added to the wells of the plate. SIVmac251 virus, diluted 1:25 in IL-2 GM, was subsequently added to the appropriate wells containing the compounds of interest and the cells except for controls. This virus represents the same virus preparation utilized for infection of the animals. The plate was then incubated overnight and, after extensive washing the next day, the plate was then placed back in the incubator at 37° C., 5% CO2.

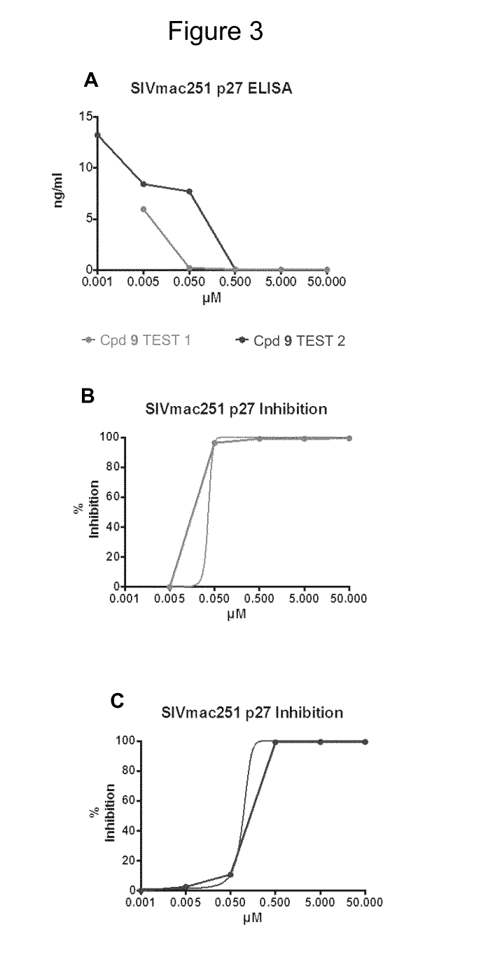

On day 4-5, of the assay, supernatants were harvested and a SIV p27 antigen capture assay was used to detect SIV p27 in the supernatant. Due to suboptimal activation of NHP PBMC in the first experiment that resulted in low cell recovery (test 1), the experiment was repeated (test 2). The results are reported FIG. 3, and demonstrated that in both experiments (test 1 light-gray line and test 2 dark-gray line) we observed a dose-dependent reduction of the amount of p27 in the supernatant of the cultures (FIG. 3a). The percentage of inhibition in each experiment is reported in FIGS. 3b and 3c for test 1 and 2, respectively.

### Example 5

### Evaluation of HIV Virus Inhibition

Several additional compounds were also tested for HIV virus inhibition, and the results are show in Table 5. P24 concentration in the supernatant was measured using a commercial ELISA assay. Toxicity was assessed by counting live cells (trypan blue exclusion)

TABLE 5
Evaluation of compounds for HIV-1 inhibition

| | p24 inhibition | Cytotoxicity | Selectivity Index |
|---|---|---|---|
| Compound | EC₅₀ (nM) | TC₅₀ (nM) | TI (TC₅₀/EC₅₀) |
| 1 | 8.1 | 436 | 54 |
| 2 | 52 | 617 | 12 |
| 3 | 1.3 | 1160 | 901 |
| 4 | 89 | 2168 | 24 |
| 5 | 2155 | ND | ND |
| 6 | 32 | ND | ND |
| 7 | 81 | ND | ND |
| 8 | 42 | ND | ND |

ND = not deterimined

### Example 6

### Evaluation of HIV-1 Replication in SupT1

SupT1 cells were used with a single-cycle HIV construct (Envelope-deficient, containing Luciferase, pseudotyped with VSV-G), where the viruses can infect cells but they cannot spread to other cells. The HIV used contains a luciferase gene, so if cells become HIV infected, they express firefly luciferase. The experiment included cells only (negative control), HIV and HIV+DMSO (positive controls), HIV+reverse transcriptase inhibitors (AZT, nevirapine (NVP)), HIV+integrase inhibitor (raltegravir (Ral)), HIV+a protease inhibitor (nelfinavir (NFV)).

SupT1 cells were grown under normal conditions and 4.0×10⁵ cells were seeded into wells of a 12-well plate (BD-Falcon) in RPMI complete media (ThermoFisher Scientific). Wells were infected with 50 μL VSVg-pseudotyped NL4-3.Luc.E⁻R⁻ virus. SupT1 cells were infected for 36 hours. Certain wells were also inoculated with various drugs and various doses of Compound 9. The samples were incubated at 37° C. for 24 h. 250 μL of cells from each well were removed; cells were pelleted and lysed with 100 μL of 1× Passive Lysis Buffer (Promega). 50 μL of cell lysate was added to a 96-well luciferase plate (BD-Falcon) and a luciferase assay was performed with 100 μL of Luciferase Assay Reagent II (Promega), 0.4 seconds between injection and integration, and 10 seconds for integration.

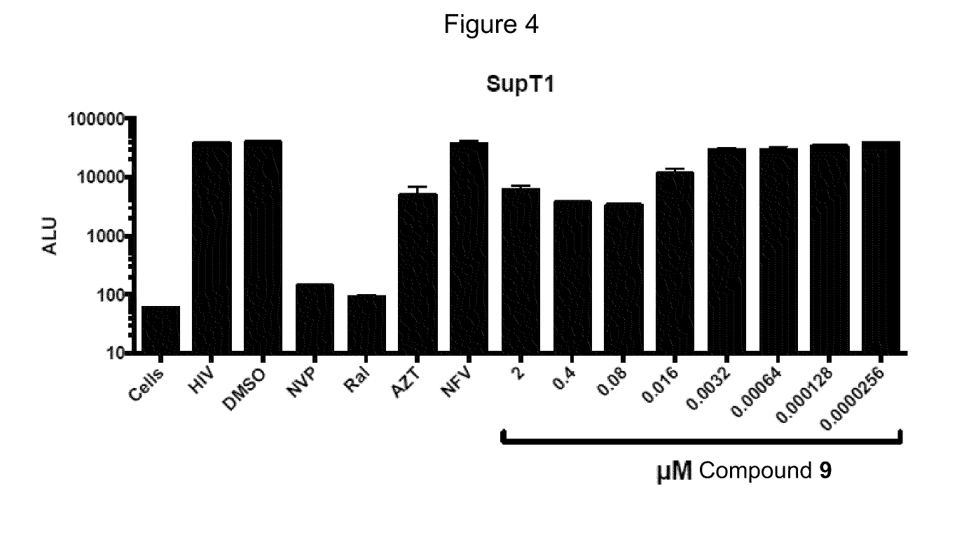

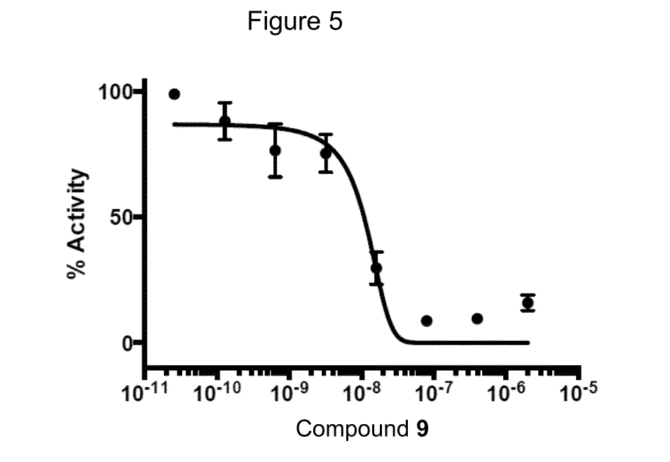

Raw data (arbitrary light units) from all of the treatments are plotted on a log₁₀ scale is shown in FIG. 4. Infection was robust with low/no background from uninfected cells. The administered drugs showed the expected activity, although AZT was not as efficacious as the others. FIG. 5 provides the % activity following Compound 9 treatment. The activity is plotted relative to DMSO control. Compound 9 potently inhibits HIV replication with an EC₅₀ of 10 nm. Without being bound to a particular theory, it is believed that Compound 9 inhibits processes prior to protein synthesis.

### Example 7

### Evaluation of HIV-1 Reactivation and Inhibition of Reactivation

Two JLAT clones (A2; 9.2) were incubated for 24 hours with tumor necrosis factor alpha (TNFα), phorbol myristate 13-acetate (PMA), Compound 9 (9, various doses), and all combinations of these three compounds. JLAT cells make GFP when HIV is reactivated from latency.

J-Lat cells (JLAT 9.2, 5×10⁵/mL) were cultured in Gibco RPMI-1640 media, supplemented with 10% (vol/vol) FCS and 5% (vol/vol) penicillin streptomycin at 37° C., 5% CO2 under sterile conditions. For HIV-1 reactivation experiments, 10⁶ cells/mL were mixed with TPA (10 nM final, unless otherwise indicated) or TNFα (5 ng/mL final) and immediately 150 μL of cell mix was dispensed into 96-well plates. Compound 9 was added to the 96-well plates in serial dilutions. Cells were incubated for 24 h before analysis by flow cytometry.

JLAT (A2) cells were incubated with the following treatments: media (negative control), DMSO (SNX control), 1 ng/mL TNFα, 2 μM PMA, or these agonists+1, 0.1, 0.01, or 0.001 Compound 9 (left to right in the attached figure). 20 h post incubation, the cells were washed, fixed, and run on a flow cytometer. Media, DMSO, TNFα, and PMA were each run in quadruplicate; all other reactions were run in singlet. >10,000 events (cells) were counted for each treatment. Data are expressed as % GFP+cells (negative cells set by gating on media alone control).

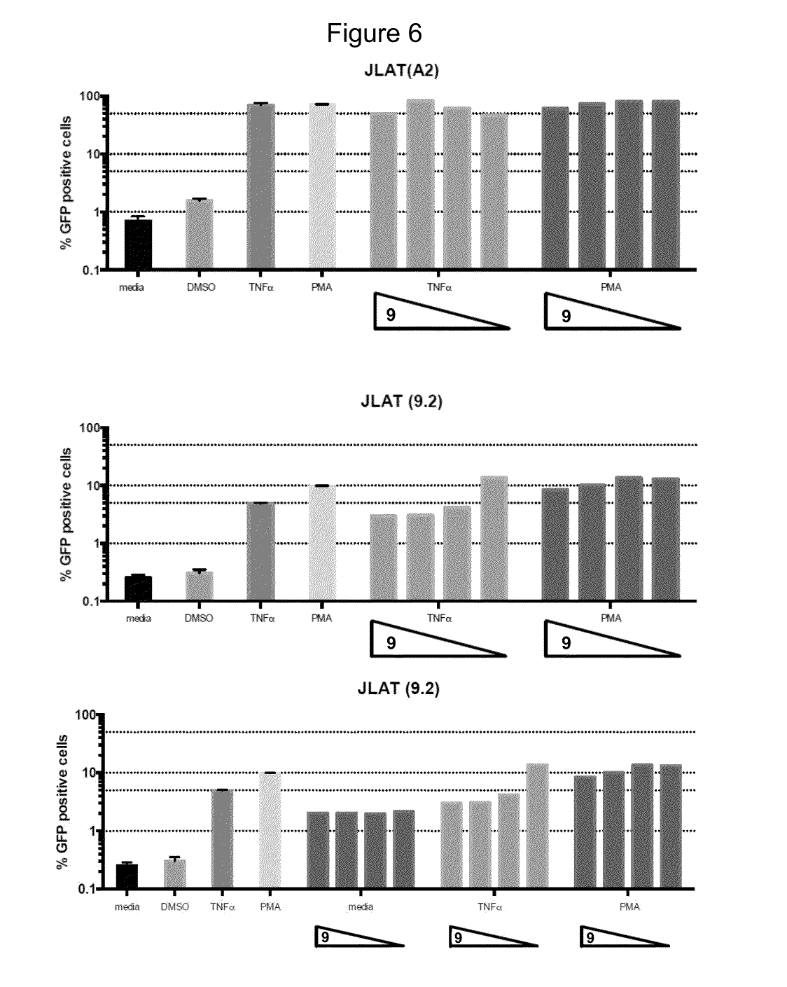

FIG. 6 shows that Compound 9 does not inhibit TNFα or PMA induced HIV-1 reactivation; neither in JLAT A2 nor in JLAT 9.2 cells. The lowest panel in FIG. 6 also shows Compound 9-mediated HIV reactivation in the JLAT 9.2 cell line model (media panel).

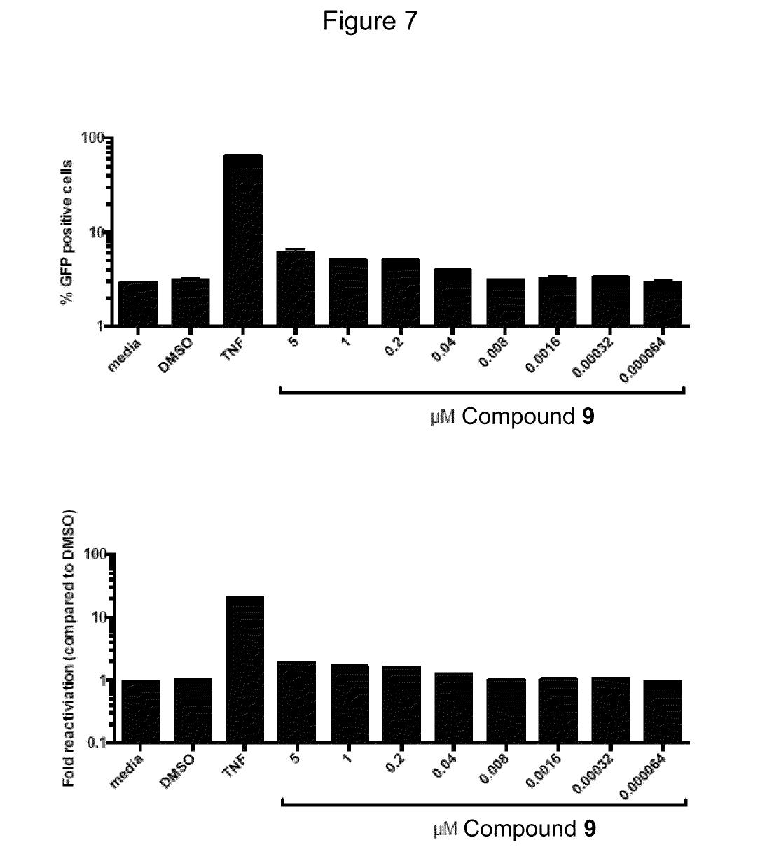

FIG. 7 shows the Compound 9-mediated HIV reactivation in JLAT (A2) model. Data represents percent GFP-positive cells (as determined by flow cytometry). DMSO (negative control), TNFα (positive control) and Compound 9 at various doses. The experiment has been repeated 4 times. Without being bound to a particular theory, it is believed that Compound 9 appears to interfere with Tat's gene silencing process with reproducible about 2-fold increase in GFP positive cells.

### Example 8

### Evaluation of TAT-Mediated Transactivation

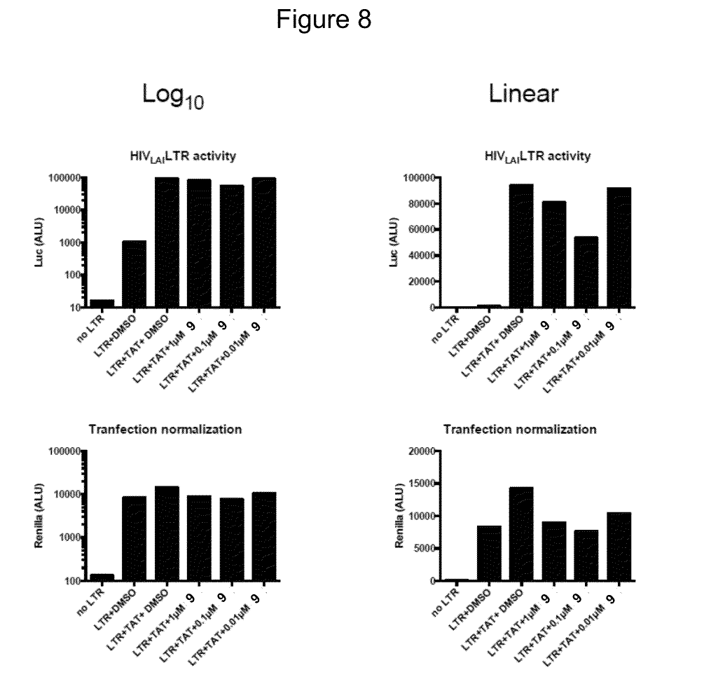

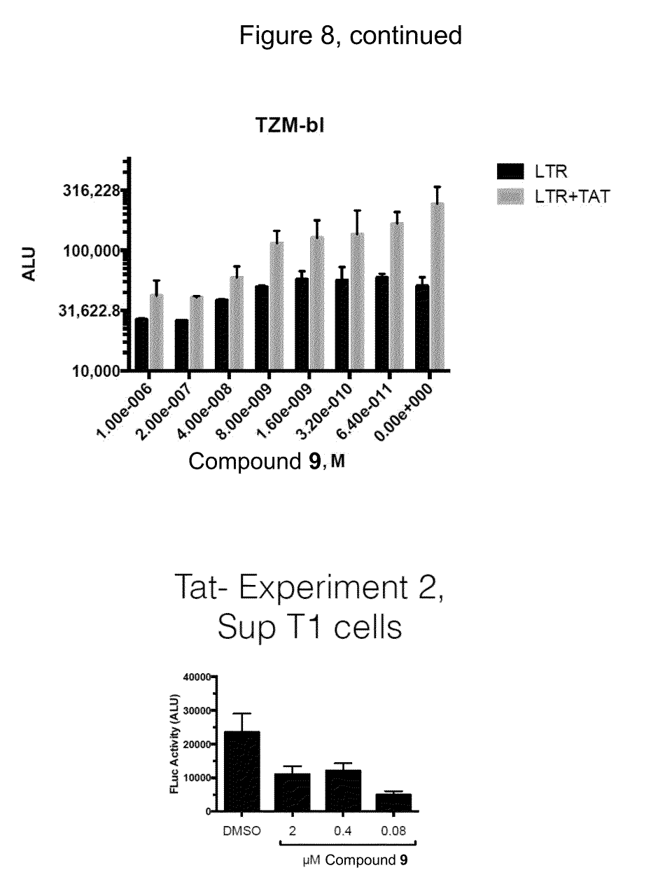

SupT1 cells were transfected with a HIV-LTR-Luc construct (LAI), +/−Tat (pcTAT, subtype B), along with a constitutively active renilla-luciferase construct (to normalize for transfection efficiency). Firefly and renilla activity were monitored (sequentially, same sample) 24 hours post transfection. DMSO was used as a negative control; and Compound 9 was added at 1, 0.1, and 0.001 μM. These reactions were done in singlet (pilot). TZM-bl cells are HeLa derived cells that contain CD4, CCR5, and an integrated HIV promoter (LTR) fused to firefly luciferase. Untransfected cells (LTR) or Tat-transfected cells (LTR+TAT) were incubated with Compound 9 at the indicated doses at the time of transfection. DMSO was used as a negative control. Luciferase activity was measured 20 hours post-transfection using a commercially-available luciferase substrate (Promega) and the results are shown in FIG. 8. These results show no signal without LTR, a substantial signal with LTR alone (about 100-fold), and a robust Tat-mediated activation of the HIV-LTR promoter (about 100-fold). Repeat results are illustrated in Tat-Experiment 2 graph.

### Example 9

### Evaluation of HIV Integration in ALU-PCR Assay

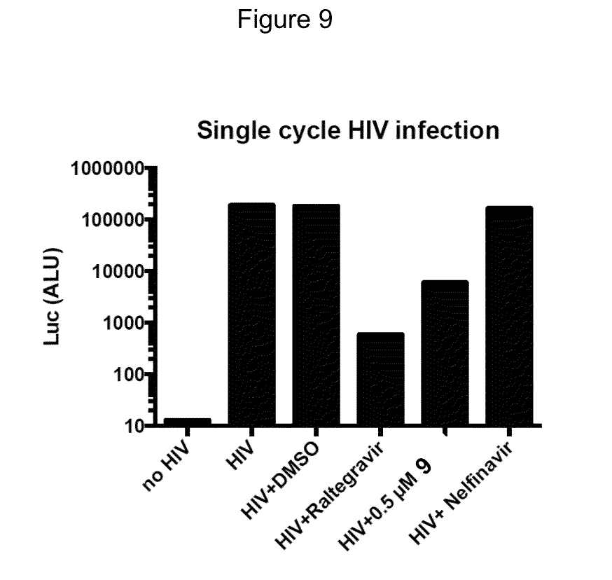

HIV-integration was evaluated in a cell based assay with ALU-PCR read out. Using single cycle virus (Env-deficient, containing Luc, pseudotyped with VSV-g), SupT1 cells were infected for 36 hours. Cells were treated with DMSO, Raltegravir (integrase inhibitor), 500 nM Compound 9, or Nelfinavir (protease inhibitor). It was expected that Raltegravir should affect both Luc and ALU-PCR data and Nelfinavir should not (downstream of both). Cells were split in half-one part for Luc-assays, and one part for ALU-PCR. The luc assays, which served as a positive control for both the infection and the drug treatments, worked as expected. The results are presented in FIG. 9 and show that Compound 9 inhibits single cycle HIV (at least 90% reduction) and there is consistently a clear, robust increase in ALU-HIV signal (comparing uninfected to HIV-infected cells).

### Example 10

### Evaluation of HIV Integration in 2LTR Assay

HIV-integration was also evaluated in 2LTR assay. The 2LTR assay determines the amount a non-integrated circular form of the HIV genome that can accumulate in the host cell. Inhibitors that prevent certain steps of viral replication cycle can lead to increased expression levels of the 2LTR form compared to the normal amounts that form during viral replication. As an integrase inhibitor, raltegravir (RAL) is expected to increase 2LTR formation. No HIV and nevirapine (NEV) are both expected to have negligible levels of 2LTR expression.

SupT1 cells were grown under normal conditions and 4.0×10⁵ cells were seeded into wells of a 12-well plate in RPMI complete media. Wells were infected with 50 μL VSVg-pseudotyped NL4-3.Luc.E⁻R⁻ virus. Certain wells were also inoculated with either 1 μM Raltegravir (NIH AIDS Reagent Program) or 500 nM Compound 9. The samples were incubated at 37° C. for 24 h. 250 μL of cells from each well were removed; cells were pelleted and lysed with 100 μL of 1× Passive Lysis Buffer (Promega). 50 μL of cell lysate was added to a 96-well luciferase plate (BD-Falcon) and a luciferase assay was performed with 100 μL of Luciferase Assay Reagent II (Promega), 0.4 seconds between injection and integration, and 10 seconds for integration. DNA was extracted from the remaining cells using the DNeasy Blood and Tissue kit (QIAgen). Real-time PCR was utilized to measure the amount of 2LTR production with the different drug treatments based as published by Suzuki et al. (“Quantitative Analysis of Human Immunodeficiency Virus Type 1 DNA Dynamics by Real-Time PCR: Integration Efficiency in Stimulated and Unstimulated Peripheral Blood Mononuclear Cells;” Virus Genes 27(2):177-188 (2003)). Primers for the 2-LTR circle were: 2LTR-S(5′-CCC TCA GAC CCT TTT AGT CAG TG-3′) and 2LTR-AS (5′-TGG TGT GTA GTT CTG CCA ATC A-3′). SYBR iQ Supermix (BioRad) was used according to specifications along with 220 ng template. Cycling conditions were 50° C. 2 minutes and 95° C. 10 minutes for hot-start; followed by 40 cycles of 95° C. for 15 seconds and 60° C. for 1 minute. Results were normalized by quantification of genomic GAPDH using the primers: GAPDH-F (5′-GGG AAA CTG TGG CGT GAT-3′) and GAPDH-R (5′-GGA GGA GTG GGT GTC GTT-3′).

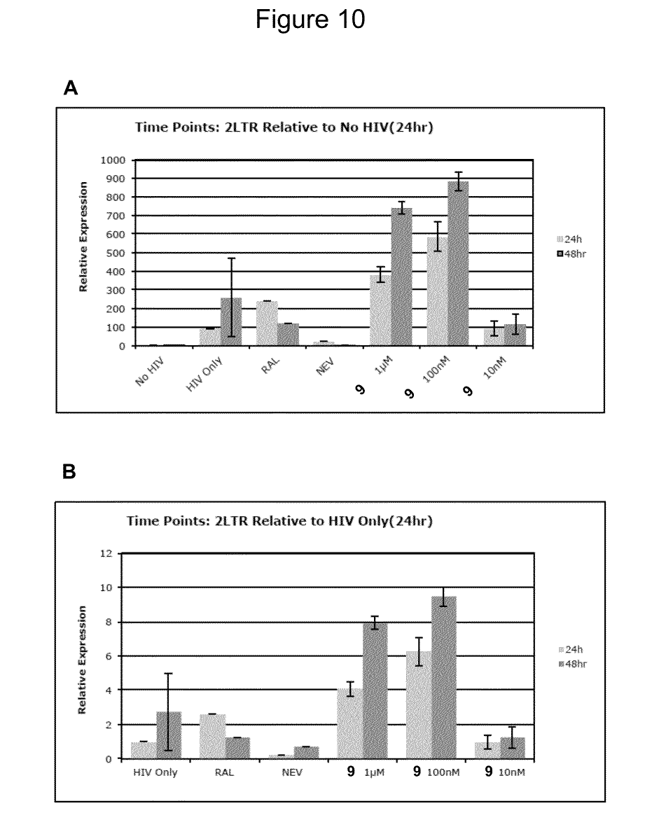

The results are presented in FIG. 10. Both graphs illustrate that 1 μM and 100 nM concentrations of Compound 9 had the highest expression levels of 2LTR over all other samples. NEV containing samples and no HIV samples had the expected low expression of 2LTR. 10 nM Compound 9 for both time points had expression levels similar to HIV Only.

### Example 11

### Time-of-Drug Addition Experiment

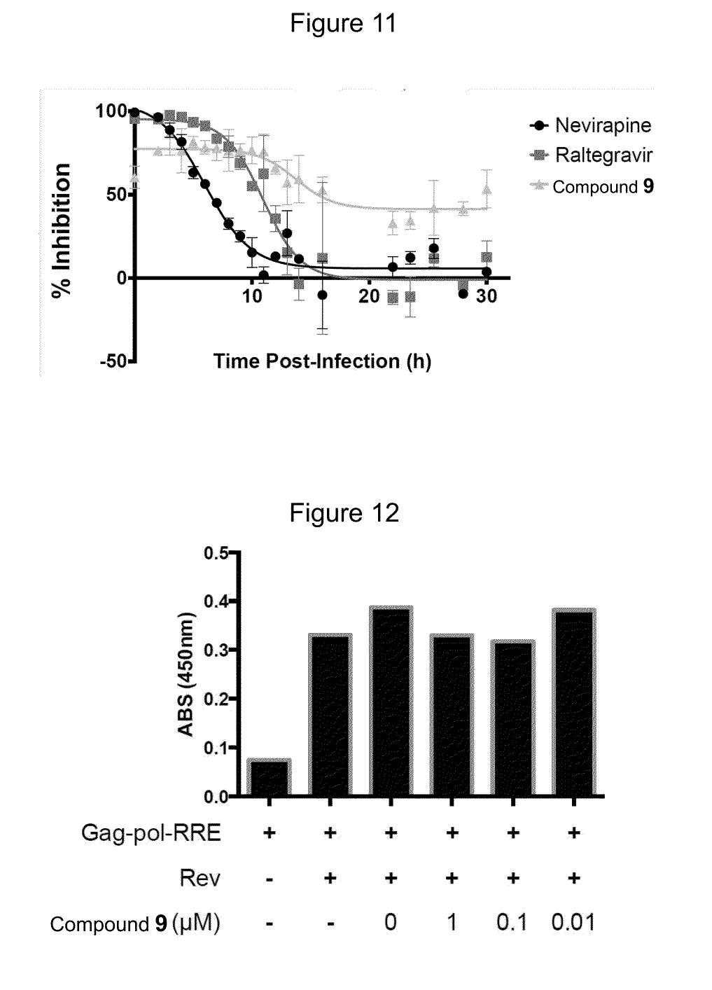

A Time-of-drug Addition approach may be useful to identify antiviral processes interfered by a compound by comparing to drugs with known mechanisms of action. TZM-bl cells were grown under normal conditions and cells were diluted to 4×10⁵ cells/mL in 13.0 mL of ice-cold DMEM. 60 μL of this stock were seeded into 3 wells of a 96-well plate. 10 mL of this cell stock was mixed with 1 mL of NL4-3 virus and centrifuged at 1,200×g, 4° C., for 80 minutes. Cells were washed two times with ice-cold PBS and re-suspended in 8.8 mL of ice-cold DMEM. 150 μL of pre-warmed (37° C.) media was added to each well for 22 columns of 96-well plates and the three wells containing cells only. Infection was started by adding 50 μL of cells and virus to each well. A master drug plate was prepared by adding 400 μL of complete media (DMEM) to a single column of a 96-well plate, and then adding 4 μL of 100 μM Nevirapine to two wells, 4 μL of 100 μM Raltegravir to three wells, and 2 μL of 50 μM Compound 9 to three wells. Using a multi-channel pipet, 2 μL of the stock drugs was removed from the drug plate and added to the corresponding time point column of the plates containing cells and virus. Drug was added to cells at every hour from 0 to 14 hours, then 16 hours, and every two hours from 22 to 30 hours post-infection. At 48 hours post-infection, media was removed from wells and 100 μL of 1× Passive Lysis Buffer was added to each well, and plates were incubated at −80° C. 50 μL of cell lysate was added to 96-well luciferase assay plates and luciferase assay was performed with 100 μL of Luciferase Assay Reagent II (Promega), 2 seconds between injection and integration, and 10 seconds for integration. Values for the cell only wells were averaged and subtracted from all other values. Percent inhibition was calculated for each drug using an average of luciferase values from 16 wells containing infected cells only and not treated with drug as the normal activity for the calculation. The results are shown in FIG. 11.

### Example 11

### Evaluating Effect on Virus Like Particles (VLP) Production

293T Cells were transfected with a gag-pol-RRE construct. When Rev is present and/or functioning, Gag is produced, forms virus like particles (VLP), and VLP are secreted into the culture supernatant. VLP in the media are quantified using a quantitative Gag(p24) ELISA. Tested the effect of 1, 0.1, and 0.01 Compound 9 on VLP production, and the results are presented in FIG. 12. Without being bound to a particular theory, these results show that VLP production depends on Rev, and that Compound 9 does not appear to robustly inhibit Rev-dependent VLP production.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method for treating or inhibiting Human Immunodeficiency Virus (HIV) infection, or treating or inhibiting Acquired Human Immunodeficiency Syndrome (AIDS), in a subject in need thereof, the method comprising administering to the subject a therapeutically effective amount of an Hsp90 inhibitor of formula (I): 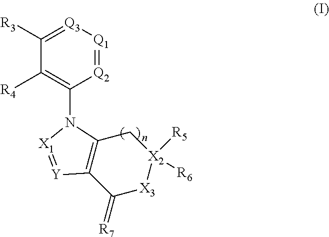
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two 0 atoms, two S atoms, or an O and S atom are not immediately adjacent each other,
wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R₀ is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is: 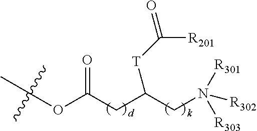
wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —O(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁,
Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 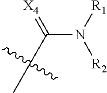
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

2. A method for treating HIV infection or treating AIDS, by inhibiting integration of HIV viral DNA into a host cell, in a subject in need thereof, the method comprising administering to the subject a therapeutically effective amount of an Hsp90 inhibitor of formula (I): 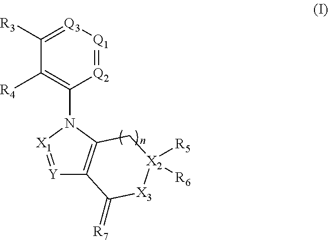
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two 0 atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R₀ is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is: 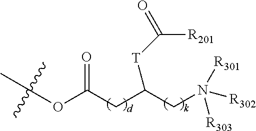
wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —O(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 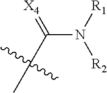
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

3. A method for treating HIV infection or treating AIDS, by inhibiting Tat-mediated transactivation of HIV DNA, in a subject in need thereof, the method comprising administering to the subject a therapeutically effective amount of an Hsp90 inhibitor of formula (I): 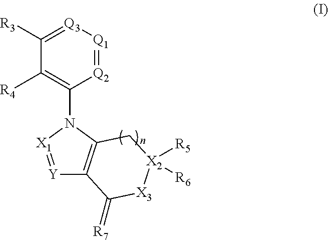
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two 0 atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R₀ is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is: 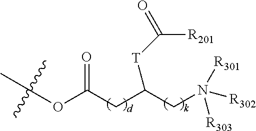
wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 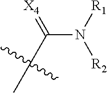
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

4. A method for treating HIV infection or treating AIDS, by:
a) reactivating a latent HIV provirus in a host cell, and b) by inhibiting integration of HIV viral DNA into a host cell and/or by inhibiting Tat-mediated transactivation of HIV DNA,
in a subject in need thereof, the method comprising administering to the subject a therapeutically effective amount of an Hsp90 inhibitor of formula (I): 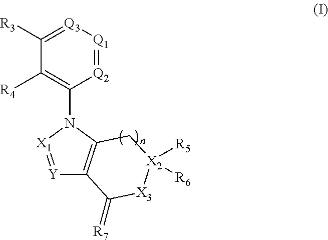
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two 0 atoms, two S atoms, or an O and S atom are not immediately adjacent each other,
wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R₀ is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is: 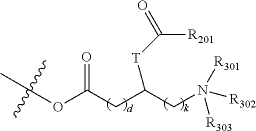
wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 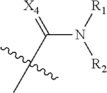
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

5. The method claim 1, wherein the Hsp90 inhibitor is selected from a compound of Table 1.

6. The method of claim 1, wherein the Hsp90 inhibitor is: 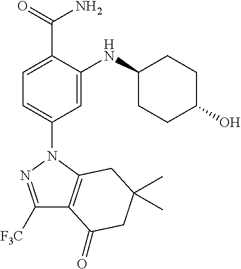 [SMILES: CC1(C)CC(=O)c2c(C(F)(F)F)nn(-c3ccc(C(N)=O)c(N[C@H]4CC[C@H](O)CC4)c3)c2C1]
4-(6,6-dimethyl-4-oxo-3-trifluoromethyl-4,5,6,7-tetrahydro-indazol-1-yl)-2-(trans-4-hydroxy-cyclohexylamino)-benzamide,
or a pharmaceutically acceptable salt thereof.

7. The method of claim 1, wherein the Hsp90 inhibitor is: 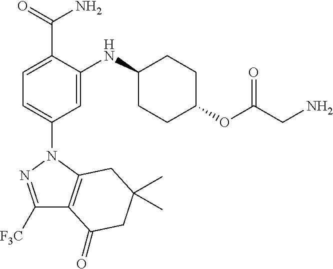 [SMILES: CC1(C)CC(=O)c2c(C(F)(F)F)nn(-c3ccc(C(N)=O)c(N[C@H]4CC[C@H](OC(=O)CN)CC4)c3)c2C1]
trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate,
or a pharmaceutically acceptable salt thereof.

8. The method of claim 1, which is treating or inhibiting a HIV infection.

9. The method of claim 8, wherein the HIV is HIV-1 or HIV-2.

10. The method of claim 1, which is treating or inhibiting AIDS.

11. The method of claim 1 further comprising administering a therapeutically effective amount of at least one other antiviral agent.

12. The method of claim 11, wherein the antiviral agent is selected from the group consisting of the nucleoside reverse transcriptase inhibitors, the non-nucleoside reverse transcriptase inhibitors and the protease inhibitors.

13. The method of claim 1, wherein the therapeutically effective amount comprises a dosage of between about 0.1 mg to about 100 mg per day.

14. A method for reactivation of latent HIV provirus in a subject in need thereof, the method comprising administering to the subject a therapeutically effective amount of an Hsp90 inhibitor of formula (I): 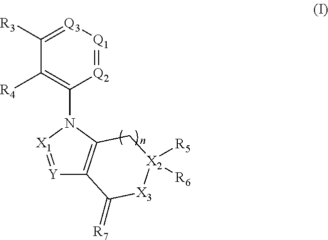
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH—aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, —O—C(O)C₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R₀ is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ is optionally substituted with a group R₅₀ where R₅₀ is: 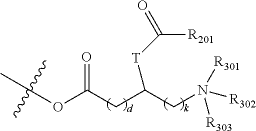
wherein
d and k are integers independently selected from 1 and 2;
R₂₀₁ is (C₁-C₆)alkyl where the alkyl is optionally substituted with (C₃-C₇)cycloalkyl, (C₂-C₆)alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₀-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, Cᵣ C₆ alkoxy, halogen, hydroxy, amino, mono- or di-(C₁-C₆)alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 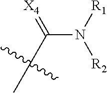
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide, wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

15. The method of claim 14, wherein the Hsp90 inhibitor is
4-(6,6-dimethyl-4-oxo-3-trifluoromethyl-4,5,6,7-tetrahydro-indazol-1-yl)-2-(trans-4-hydroxy-cyclohexylamino)-benzamide,
or a pharmaceutically acceptable salt thereof.

16. The method of claim 14, wherein the Hsp90 inhibitor is
trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate,
or a pharmaceutically acceptable salt thereof.

17. The method of claim 14, wherein the subject is a subject previously treated with an antiviral therapy.
